(19) United States
(12) Patent Application Publication
(10) Pub. No.: US 2019/0175720 A1
(43) Pub. Date: Jun. 13, 2019

(54) ZIKA VIRUS VACCINE AND COMBINATION VACCINE

(71) Applicant: Oxford University Innovation Limited, Oxford (GB)

(72) Inventors: Arturo REYES-SANDOVAL, Oxford (GB); Cesar LOPEZ-CAMA-CHO, Oxford (GB)

(21) Appl. No.: 16/321,141

(22) PCT Filed: Jul. 28, 2017
(86) PCT No.: PCT/GB2017/052220

§ 371 Date: Jan. 28, 2019

(30) Foreign Application Priority Data
Jul. 29, 2016 (GB) 1613191.4

(51) Int. Cl. A61K 39/12; C12N 7/00; A61P 31/14

(52) U.S. Cl. CPC A61K 39/12; C12N 7/00; A61P 31/14; C12N 2770/24143; A61K 2039/70; C12N 2770/24171; C12N 2770/36143; C12N 2770/36134; C12N 2770/24134

(57) ABSTRACT

The invention relates to a Zika viral vector vaccine comprising nucleic acid encoding a Zika virus structural antigen, wherein the nucleic acid encoding a Zika virus structural antigen comprises a sequence encoding Zika virus envelope DIII, or part thereof. The invention further relates to a Zika viral vector vaccine in combination with a Chikungunya viral vector vaccine.

The present invention relates to a Zika viral vector vaccine comprising nucleic acid encoding a Zika virus structural antigen, it use, and methods of treatment or prevention of Zika viral infection. Additionally, the use of the Zika viral vector vaccine in combination with a Chikungunya vaccine.

Zika virus (ZIKV) is an emerging mosquito-borne virus of the family Flaviviridae that has originally spread from Africa, through Polynesia and is now spreading rapidly throughout the Americas. Major concerns are the neurologic conditions that have been associated with this arbovirus infection, such as the Guillain-Barré syndrome documented from French Polynesia, and a concurrent 20-fold increase in the incidence of microcephaly during the ZIKV outbreak in French Polynesia and Brazil. The most serious consequence of ZIKV infection is the teratogenic effect on the developing foetus, and there is therefore an urgent need to protect women before or during pregnancy from infection by the virus. At present, there is no vaccine available, or any effective drug treatment. Given the potential hazards of drug treatment during pregnancy, a preventative vaccine would clearly be the more preferable option.

Vaccine development is a lengthy process that requires careful selection of the best candidates to provide the best protection. Every pathogen's genetic sequence inserted into a new viral vectored vaccine will produce proteins that will follow various pathways of secretion depending on the leading sequences and presence of transmembrane regions.

One of the major technical challenges for a vaccine is the production of a protein with the correct folding, able to induce antibody responses not only against linear epitopes but also against conformational ones. Therefore, various eukaryotic and prokaryotic systems have been developed for this purpose and the successful production relies on a trial and error system to produce vaccines. Recombinant viral vectors do not face this challenge due to the fact that the protein is produced inside an organism in a similar way to native viral proteins during an infection, and folding does not become an issue. However, other challenges can arise with the introduction of a gene into a viral vector, such as achieving production and secretion of high amounts of protein to stimulate robust antibody and cytotoxic responses. For this purpose, genetic sequences must be optimised and they require the addition of leading sequences to support secretion, elimination or preservation of transmembrane regions and codon optimisation for the target organism to be vaccinated with the viral vector. There is no previous publication describing the best approach to be followed to construct a new ZIKV recombinant viral vectored vaccine and various options must be considered before taking a new construct to a clinical trial.

Another technical challenge we may face is the lack of induction of robust immune responses, low protective levels against ZIKV challenge or modest neutralisation titres by immune sera.

An aim of the present invention is to provide a Zika vaccine that can provide an appropriate immune response against infection from many or all strains of Zika virus.

According to a first aspect of the present invention, there is provided a Zika viral vector vaccine comprising nucleic acid encoding a Zika virus structural antigen, wherein the nucleic acid encoding a Zika virus structural antigen comprises a sequence encoding Zika virus envelope DIII, or part thereof.

Advantageously, the DIII region is the target of neutralising antibodies to prevent virus entry into cells and immune responses can focus on these epitopes, while preventing immune responses against other structural antigens that have been implicated in severe disease, such as antibody-dependent enhancement (ADE), additionally, the expression of only DIII region would support the protein secretion and stimulation of antibodies. Viral vectors can be administered as a single dose without the requirement of any adjuvant. This simplifies vaccination, making it affordable for low- to intermediate-income countries, and at the same time making logistics for vaccination very simple. Other vaccine approaches, such as virus-like particles or inactivated viruses require multiple doses and the use of adjuvants. Multiple components drive up the basic cost-of-goods of the vaccine, making them unaffordable for developing countries. The vaccine of the present invention will be affordable for all ZIKV-endemic countries.

In one embodiment, the nucleic acid encoding a Zika virus structural antigen consists essentially of a sequence encoding Zika virus envelope DIII, or a part thereof.

In one embodiment, the nucleic acid encoding the Zika virus structural antigen comprises or consists of the sequence of SEQ ID NO: 1 (DIII consensus). The Zika virus envelope may comprise the whole Zika virus envelope DIII sequence. For example, the Zika virus envelope DIII may comprise the whole Zika virus envelope DIII sequence of SEQ ID NO: 1 (DIII consensus). Alternatively, the Zika virus envelope DIII may comprise at least 99%, 98%, 95%, 90%, 85%, 80%, 70%, 60% or 50% of the Zika virus envelope DIII sequence. In one embodiment of the second aspect, Zika virus envelope DIII comprises at least 99%, 98%, 95%, 90%, 85%, 80%, 70%, 60% or 50% of the Zika virus envelope DIII sequence of SEQ ID NO: 1 (DIII consensus).

Advantageously, the viral vaccine comprises consensus sequence that has been carefully designed using the published ZIKV genetic sequences reported in the literature. It is highly similar to the strains causing the epidemics in the Americas (at least 99%) but also shows great similarity to the Asian and African genotypes. The transgenic protein can thus be suitable for many countries where ZIKV is endemic.

The Zika virus envelope DIII may be a natural or modified variant thereof. The nucleic acid encoding the Zika virus envelope DIII may be a natural or modified variant thereof. In particular, the skilled person will understand that some modifications or variants of a sequence may provide the same or substantially similar immunogenic function as the unmodified sequence (i.e. the Zika virus envelope DIII encoding sequence herein). Modifications may comprise of nucleic acid encoding the Zika virus envelope DIII, for example as encoded be SEQ ID NO: 1 (DIII consensus), with amino acid residue additions, substitutions, or deletions. In one embodiment, the modification may encode for no more than 20 amino acid residue additions, substitutions, or deletions. In another embodiment, the modification may encode for no more than 15 amino acid residue additions, substitutions, or deletions. In another embodiment, the modification may encode for no more than 10 amino acid residue additions, substitutions, or deletions. In another embodiment, the modification may encode for no more than 8 amino acid residue additions, substitutions, or deletions. In another embodiment, the modification may encode for no more than 6 amino acid residue additions, substitutions, or deletions. In another embodiment, the modification may encode for no more than 5 amino acid residue additions, substitutions, or deletions. In another embodiment, the modification may encode for no more than 4 amino acid residue additions, substitutions, or deletions. In another embodiment, the modification may encode for no more than 3 amino acid residue additions, substitutions, or deletions. In another embodiment, the modification may encode for no more than 2 amino acid residue additions, substitutions, or deletions. In another embodiment, the modification may encode for no more than 1 amino acid residue addition, substitution, or deletion. The amino acid residue additions, substitutions, or deletions may involve consecutive amino acids, multiple groups of amino acids, or non-consecutive amino acid residues, or combinations thereof. Modifications may comprise conservative substitutions of nucleotides using codon redundancy to encode the same Zika virus envelope DIII, or part thereof, as encoded by SEQ ID NO: 1 (DIII consensus) [nt seq]. The nucleic acid may encode Zika virus envelope DIII, or part thereof, according to SEQ ID NO: 2 (DIII consensus aa sequence).

Variants of the nucleic acid encoding the Zika virus envelope DIII may comprise or consist of a sequence having at least 80% identity with SEQ ID NO: 1. Alternatively, variants of the nucleic acid encoding the Zika virus envelope DIII may comprise or consist of a sequence having at least 85% identity with SEQ ID NO: 1. Alternatively, variants of the nucleic acid encoding the Zika virus envelope DIII may comprise or consist of a sequence having at least 90% identity with SEQ ID NO: 1. Alternatively, variants of the nucleic acid encoding the Zika virus envelope DIII may comprise or consist of a sequence having at least 95% identity with SEQ ID NO: 1. Alternatively, variants of the nucleic acid encoding the Zika virus envelope DIII may comprise or consist of a sequence having at least 98% identity with SEQ ID NO: 1. Alternatively, variants of the nucleic acid encoding the Zika virus envelope DIII may comprise or consist of a sequence having at least 99% identity with SEQ ID NO: 1. Alternatively, variants of the nucleic acid encoding the Zika virus envelope DIII may comprise or consist of a sequence having at least 99.5% identity with SEQ ID NO: 1. The sequence identity may be over at least 50 consecutive nucleotides of SEQ ID NO: 1. Alternatively, the sequence identity may be over at least 80 consecutive nucleotides of SEQ ID NO: 1. Alternatively, the sequence identity may be over at least 100 consecutive nucleotides of SEQ ID NO: 1. Alternatively, the sequence identity may be over at least 150 consecutive nucleotides of SEQ ID NO: 1. Alternatively, the sequence identity may be over at least 200 consecutive nucleotides of SEQ ID NO: 1. Alternatively, the sequence identity may be over at least 300 consecutive nucleotides of SEQ ID NO: 1. Alternatively, the sequence identity may be over the whole nucleotide sequence of SEQ ID NO: 1.

In another embodiment, variants of Zika virus envelope DIII may comprise or consist of a truncated sequence of the Zika virus envelope DIII encoding sequence of SEQ ID NO: 1 (DIII consensus). For example, the sequence of SEQ ID NO: 1 herein may be truncated and still provide immunogenicity. The truncated sequence may comprise at least 20 amino acids of the sequence of Zika virus envelope DIII encoded by the sequence of SEQ ID NO: 1 (DIII consensus). The truncated sequence may comprise at least 30 amino acids of the sequence of Zika virus envelope DIII encoded by the sequence of SEQ ID NO: 1 (DIII consensus). The truncated sequence may comprise at least 40 amino acids of the sequence of Zika virus envelope DIII encoded by the sequence of SEQ ID NO: 1 (DIII consensus). The truncated sequence may comprise at least 50 amino acids of the sequence of Zika virus envelope DIII encoded by the sequence of SEQ ID NO: 1 (DIII consensus). Alternatively, the truncated sequence may comprise at least 100 amino acids of the sequence of Zika virus envelope DIII encoded by the sequence of SEQ ID NO: 1 (DIII consensus).

Additionally or alternatively, the Zika viral vector vaccine may not comprise sequence encoding Zika virus TM (transmembrane) domain or part thereof. Additionally or alternatively, the Zika viral vector vaccine may not comprise sequence encoding Zika virus prM domain or part thereof.

In one embodiment, the Zika viral vector vaccine may not comprise sequence encoding a Zika virus non-structural domain or part(s) thereof. In an alternative embodiment, the Zika viral vector vaccine may comprise sequence encoding a Zika virus non-structural domain or part(s) thereof.

According to a second aspect of the present invention, there is provided a Zika viral vector vaccine comprising nucleic acid encoding a Zika virus structural antigen, wherein the nucleic acid encoding a Zika virus structural antigen comprises a sequence encoding at least part of the Zika virus prM, and a sequence encoding at least part of the Zika virus envelope protein.

Advantageously, a sequence containing the PrM and Env regions would benefit of the inclusion of additional epitopes to expand additional T-cell responses, including CD4 help to support antibodies, or CD8 cells to eliminate infected cells. Similarly, it could include additional B-cell epitopes to stimulate broader antibody responses against Zika virus.

In one embodiment of the second aspect, the nucleic acid encoding a Zika virus structural antigen consists essentially of a sequence encoding Zika virus envelope, or a part thereof, and prM, or part thereof.

The Zika virus envelope may comprise the sequence of SEQ ID NO: 3 (ZENV_noTM), or a part thereof. In one embodiment of the second aspect, the nucleic acid encoding the Zika virus structural antigen comprises or consists of the sequence of SEQ ID NO: 7 (ZprMENV_noTM) (SEQ ID NO: 7), or part(s) thereof, or variant thereof. In another embodiment, the nucleic acid encoding the Zika virus structural antigen comprises or consists of SEQ ID NO: 9 (ZprMENV_TM), or part(s) thereof, or a variant thereof. A variant of the sequence of SEQ ID NO: 7 (ZprMENV_noTM) may comprise at least 99%, 98%, 95%, 90%, 85%, 80%, 70%, 60% or 50% sequence identity with SEQ ID NO: 7. A variant of the sequence of SEQ ID NO: 9 (ZprMENV_TM) may comprise at least 99%, 98%, 95%, 90%, 85%, 80%, 70%, 60% or 50% sequence identity with SEQ ID NO: 9.

The Zika virus envelope may comprise the whole envelope sequence. The Zika virus envelope may comprise at least two of the DI, DII or DII domains, or parts thereof, of the envelope sequence. The Zika virus envelope may comprise at part of all DI, DII and DII domains of the envelope sequence. For example, the Zika virus envelope may comprise the whole envelope sequence of SEQ ID NO: 3 (ZENV_noTM). Alternatively, the Zika virus envelope may comprise at least 99%, 98%, 95%, 90%, 85%, 80%, 70%, 60% or 50% of the envelope sequence. In one embodiment of the second aspect, the Zika virus envelope comprises at least 99%, 98%, 95%, 90%, 85%, 80%, 70%, 60% or 50% of the envelope sequence of SEQ ID NO: 3 (ZENV_noTM).

The Zika virus envelope may be a natural or modified variant thereof. The nucleic acid encoding the Zika virus envelope may be a natural or modified variant thereof. In particular, the skilled person will understand that some modifications or variants of a sequence may provide the same or substantially similar immunogenic function as the unmodified sequence (i.e. the Zika virus envelope encoding sequence herein). Modifications may comprise of nucleic acid encoding the Zika virus envelope, for example as encoded be SEQ ID NO: 3 (ZENV_noTM), with amino acid residue additions, substitutions, or deletions. In one embodiment, the modification may encode for no more than 20 amino acid residue additions, substitutions, or deletions. In another embodiment, the modification may encode for no more than 15 amino acid residue additions, substitutions, or deletions. In another embodiment, the modification may encode for no more than 10 amino acid residue additions, substitutions, or deletions. In another embodiment, the modification may encode for no more than 8 amino acid residue additions, substitutions, or deletions. In another embodiment, the modification may encode for no more than 6 amino acid residue additions, substitutions, or deletions. In another embodiment, the modification may encode for no more than 5 amino acid residue additions, substitutions, or deletions. In another embodiment, the modification may encode for no more than 4 amino acid residue additions, substitutions, or deletions. In another embodiment, the modification may encode for no more than 3 amino acid residue additions, substitutions, or deletions. In another embodiment, the modification may encode for no more than 2 amino acid residue additions, substitutions, or deletions. In another embodiment, the modification may encode for no more than 1 amino acid residue addition, substitution, or deletion. The amino acid residue additions, substitutions, or deletions may involve consecutive amino acids, multiple groups of amino acids, or non-consecutive amino acid residues, or combinations thereof. Modifications may comprise conservative substitutions of nucleotides using codon redundancy to encode the same Zika virus envelope, or part thereof, as encoded by SEQ ID NO: 3(ZENV_noTM). The nucleic acid may encode Zika virus envelope, or part thereof, according to SEQ ID NO: 4 (ZENV_noTM aa sequence).

Variants of the nucleic acid encoding the Zika virus envelope may comprise or consist of a sequence having at least 80% identity with SEQ ID NO: 3. Alternatively, variants of the nucleic acid encoding the Zika virus envelope may comprise or consist of a sequence having at least 85% identity with SEQ ID NO: 3. Alternatively, variants of the nucleic acid encoding the Zika virus envelope may comprise or consist of a sequence having at least 90% identity with SEQ ID NO: 3. Alternatively, variants of the nucleic acid encoding the Zika virus envelope may comprise or consist of a sequence having at least 95% identity with SEQ ID NO: 3. Alternatively, variants of the nucleic acid encoding the Zika virus envelope may comprise or consist of a sequence having at least 98% identity with SEQ ID NO: 3. Alternatively, variants of the nucleic acid encoding the Zika virus envelope may comprise or consist of a sequence having at least 99% identity with SEQ ID NO: 3. Alternatively, variants of the nucleic acid encoding the Zika virus envelope may comprise or consist of a sequence having at least 99.5% identity with SEQ ID NO: 3. The sequence identity may be over at least 50 consecutive nucleotides of SEQ ID NO: 3. Alternatively, the sequence identity may be over at least 80 consecutive nucleotides of SEQ ID NO: 3. Alternatively, the sequence identity may be over at least 100 consecutive nucleotides of SEQ ID NO: 3. Alternatively, the sequence identity may be over at least 150 consecutive nucleotides of SEQ ID NO: 3. Alternatively, the sequence identity may be over at least 200 consecutive nucleotides of SEQ ID NO: 3. Alternatively, the sequence identity may be over at least 300 consecutive nucleotides of SEQ ID NO: 3. Alternatively, the sequence identity may be over at least 500 consecutive nucleotides of SEQ ID NO: 3. Alternatively, the sequence identity may be over at least 800 consecutive nucleotides of SEQ ID NO: 3. Alternatively, the sequence identity may be over at least 1000 consecutive nucleotides of SEQ ID NO: 3. Alternatively, the sequence identity may be over the whole nucleotide sequence of SEQ ID NO: 3.

In another embodiment, variants of Zika virus envelope may comprise or consist of a truncated sequence of the Zika virus envelope encoding sequence of SEQ ID NO: 3. For example, the sequence of SEQ ID NO. 3 herein may be truncated and still provide immunogenicity. The truncated sequence may comprise at least 20 amino acids of the sequence of Zika virus envelope encoded by the sequence of SEQ ID NO: 3. The truncated sequence may comprise at least 30 amino acids of the sequence of Zika virus envelope encoded by the sequence of SEQ ID NO: 3. The truncated sequence may comprise at least 40 amino acids of the sequence of Zika virus envelope encoded by the sequence of SEQ ID NO: 3. The truncated sequence may comprise at least 50 amino acids of the sequence of Zika virus envelope encoded by the sequence of SEQ ID NO: 3. Alternatively, the truncated sequence may comprise at least 100 amino acids of the sequence of Zika virus envelope encoded by the sequence of SEQ ID NO: 3.

In one embodiment the nucleic acid may encode Zika NS2b and/or NS3, or parts thereof. In one embodiment the nucleic acid may encode capsid, prM, Env, NS2B and NS3, or parts thereof. In one embodiment the nucleic acid encoding the Zika virus structural antigen comprises or consists of the sequence of SEQ ID NO: 18 (CprME/NS), or part(s) thereof, or a variant thereof. In one embodiment the nucleic acid encodes a polypeptide of the sequence of SEQ ID NO: 19 (CprME/NS), or part(s) thereof, or a variant thereof. A variant of the sequence of SEQ ID NO: 18 (CprME/NS) may comprise at least 99%, 98%, 95%, 90%, 85%, 80%, 70%, 60% or 50% sequence identity with SEQ ID NO: 18. A variant of the sequence of SEQ ID NO: 19 (CprME/NS) may comprise at least 99%, 98%, 95%, 90%, 85%, 80%, 70%, 60% or 50% sequence identity with SEQ ID NO: 19.

The provision of NS2B and/or NS3 will help Capsid to cleave and release prM and Env to the endoplasmic reticulum, aiding to form a Viral Like Particle. In addition, T cell responses elicited by NS3 can be additive to vaccine efficacy.

The Zika virus prM may comprise the whole prM sequence. For example, the Zika virus prM may comprise the whole prM sequence of SEQ ID NO: 13. Alternatively, the Zika virus prM may comprise at least 99%, 98%, 95%, 90%, 85%, 80%, 70%, 60% or 50% of the prM sequence. In one embodiment of the second aspect, the Zika virus prM comprises at least 99%, 98%, 95%, 90%, 85%, 80%, 70%, 60% or 50% of the envelope sequence of SEQ ID NO: 13.

The Zika virus prM may be a natural or modified variant thereof. The nucleic acid encoding the Zika virus prM may be a natural or modified variant thereof. In particular, the skilled person will understand that some modifications or variants of a sequence may provide the same or substantially similar immunogenic function as the unmodified sequence (i.e. the Zika virus prM encoding sequence herein). Modifications may comprise of nucleic acid encoding the Zika virus prM, for example the Zika virus prM of the sequence of SEQ ID NO: 13, with amino acid residue additions, substitutions, or deletions. In one embodiment, the modification may encode for no more than 20 amino acid residue additions, substitutions, or deletions. In another embodiment, the modification may encode for no more than 15 amino acid residue additions, substitutions, or deletions. In another embodiment, the modification may encode for no more than 10 amino acid residue additions, substitutions, or deletions. In another embodiment, the modification may encode for no more than 8 amino acid residue additions, substitutions, or deletions. In another embodiment, the modification may encode for no more than 6 amino acid residue additions, substitutions, or deletions. In another embodiment, the modification may encode for no more than 5 amino acid residue additions, substitutions, or deletions. In another embodiment, the modification may encode for no more than 4 amino acid residue additions, substitutions, or deletions. In another embodiment, the modification may encode for no more than 3 amino acid residue additions, substitutions, or deletions. In another embodiment, the modification may encode for no more than 2 amino acid residue additions, substitutions, or deletions. In another embodiment, the modification may encode for no more than 1 amino acid residue addition, substitution, or deletion. The amino acid residue additions, substitutions, or deletions may involve consecutive amino acids, multiple groups of amino acids, or non-consecutive amino acid residues, or combinations thereof. Modifications may comprise conservative substitutions of nucleotides using codon redundancy to encode the same Zika virus prM, or part thereof, of SEQ ID NO: 13. The nucleic acid may encode Zika virus prM, or part thereof, according to SEQ ID NO: 13 (PrM aa sequence).

Variants of the nucleic acid encoding the Zika virus prM may comprise or consist of a nucleic acid encoding a sequence having at least 80% identity with SEQ ID NO: 13. Alternatively, variants of the nucleic acid encoding the Zika virus prM may comprise or consist of nucleic acid encoding a sequence having at least 85% identity with SEQ ID NO: 13. Alternatively, variants of the nucleic acid encoding the Zika virus prM may comprise or consist of nucleic acid encoding a sequence having at least 90% identity with SEQ ID NO: 13. Alternatively, variants of the nucleic acid encoding the Zika virus prM may comprise or consist of nucleic acid encoding a sequence having at least 95% identity with SEQ ID NO: 13. Alternatively, variants of the nucleic acid encoding the Zika virus prM may comprise or consist of nucleic acid encoding a sequence having at least 98% identity with SEQ ID NO: 13. Alternatively, variants of the nucleic acid encoding the Zika virus prM may comprise or consist of nucleic acid encoding a sequence having at least 99% identity with SEQ ID NO: 13. Alternatively, variants of the nucleic acid encoding the Zika virus prM may comprise or consist of nucleic acid encoding a sequence having at least 99.5% identity with SEQ ID NO: 13. The sequence identity may be over at least 50 consecutive nucleotides of nucleic acid encoding SEQ ID NO: 13. Alternatively, the sequence identity may be over at least 80 consecutive nucleotides of nucleic acid encoding SEQ ID NO: 13. Alternatively, the sequence identity may be over at least 100 consecutive nucleotides of nucleic acid encoding SEQ ID NO: 13. Alternatively, the sequence identity may be over at least 150 consecutive nucleotides of nucleic acid encoding SEQ ID NO: 13. Alternatively, the sequence identity may be over at least 200 consecutive nucleotides of nucleic acid encoding SEQ ID NO: 13. Alternatively, the sequence identity may be over at least 300 consecutive nucleotides of nucleic acid encoding SEQ ID NO: 13. Alternatively, the sequence identity may be over at least 500 consecutive nucleotides of nucleic acid encoding SEQ ID NO: 13. Alternatively, the sequence identity may be over at least 800 consecutive nucleotides of nucleic acid encoding SEQ ID NO: 13. Alternatively, the sequence identity may be over at least 1000 consecutive nucleotides of nucleic acid encoding SEQ ID NO: 13. Alternatively, the sequence identity may be over the whole nucleotide sequence of nucleic acid encoding SEQ ID NO: 13.

In another embodiment, variants of Zika virus prM may comprise or consist of a truncated sequence of the Zika virus prM sequence of SEQ ID NO: 13. For example, the sequence of SEQ ID NO: 13 herein may be truncated and still provide immunogenicity. The truncated sequence may comprise at least 20 amino acids of the sequence of Zika virus prM sequence of SEQ ID NO: 13. The truncated sequence may comprise at least 30 amino acids of the sequence of Zika virus prM sequence of SEQ ID NO: 13. The truncated sequence may comprise at least 40 amino acids of the sequence of Zika virus prM sequence of SEQ ID NO: 13. The truncated sequence may comprise at least 50 amino acids of the sequence of Zika virus prM sequence of SEQ ID NO: 13. Alternatively, the truncated sequence may comprise at least 100 amino acids of the sequence of Zika virus prM sequence of SEQ ID NO: 13.

The Zika viral vector vaccine of the second aspect may not comprise sequence encoding Zika virus TM (transmembrane) domain or part thereof.

In one embodiment of the second aspect, the Zika viral vector vaccine may not comprise sequence encoding a Zika virus non-structural domain or part(s) thereof. In an alternative embodiment, the Zika viral vector vaccine may comprise sequence encoding a Zika virus non-structural domain or part(s) thereof.

In one embodiment of the first and/or second aspect of the invention, the Zika viral vector vaccine of the invention may further encode a peptide signal, which could be a peptide adjuvant. The peptide signal may comprise a secretion signal peptide sequence. For example, the peptide signal may comprise the endogenous Zika peptide signal (which is located between Capsid and Envelope). The peptide signal, such as the endogenous Zika peptide signal, may improve secretion of the antigen and provide better antibody response. In one embodiment of the first and/or second aspect of the invention, the Zika viral vector vaccine of the invention may further encode a peptide adjuvant, such as a TPA (tissue plasminogen activator) sequence, or functional variants thereof. The TPA may comprise or consist of the sequence: MDAMKRGLCCVLLLCGAVFVSPSQEIHARFRR (SEQ ID NO: 11, or a functional variant thereof. In one embodiment, the peptide adjuvant may comprise a Shark invariant chain, for example of the sequence SLLWGGVTVLAAMLIAGQVASSVVFLV (SEQ ID NO: 12), or a functional variant thereof. The peptide adjuvant may be encoded N-terminal on the antigen of the invention. A functional variant of a peptide adjuvant may be a truncated or mutated peptide variant, which can still function as an adjuvant, for example a truncated or mutated variant of the TPA or shark invariant chain, which still function as an adjuvant. The skilled person will appreciate that 1, 2, 3, 4, 5 or more amino acid residues may be substituted, added or removed without affecting function. For example, conservative substitutions may be considered.

According to the first or second aspect, the viral vector may comprise nucleic acid encoding non-Zika viral protein, such as adenovirus protein(s) or MVA protein(s). According to the first or second aspect, the viral vector may comprise a virus, or parts thereof. The viral vector may comprise an adenovirus, such as a simian adenovirus. The viral vector may comprise an adenovirus when used in a prime vaccine of a prime boost regime. The viral vector may comprise ChAdOx1 (a group E simian adenovirus, like the AdCh63 vector used safely in malaria trials). The viral vector may comprise AdCh63. The viral vector may comprise AdC3 or AdH6. The viral vector may be a human serotype. The viral vector may comprise Modified Vaccinia Ankara (MVA). The viral vector may comprise MVA when used as a vaccine boost in a prime boost regime. The viral vector may comprise Adeno-associated virus (AAV) or lentivirus. The viral vector may be an attenuated viral vector. The protein encoding sequence of the invention may be cloned into any suitable viral vector that is known to elicit good immune response. Suitable viral vectors have been described in Dicks et al (Vaccine. 2015 Feb. 25; 33(9): 1121-8. doi: 10.1016/j.vaccine.2015.01.042. Epub 2015 Jan. 25), Antrobus et al (Mol Ther. 2014 Mar;22(3):668-74. doi: 10.1038/mt.2013.284. Epub 2013 Dec. 30.), and (Warimwe et al. (Virol J. 2013 Dec. 5; 10:349. doi: 10.1186/1743-422X-10-349), which are incorporated herein by reference.

The PrM may be provided N-terminal to the envelope. Additionally wherein a peptide adjuvant is used such as tPA, the peptide adjuvant may be N-terminal, i.e. to the PrM and/or envelope sequence.

In one embodiment, the Zika virus structural antigen is expressed as a non-secreting protein in the cell, supporting the stimulation of cytotoxic T cells.

The Zika virus structural antigen may be immunogenic. The Zika virus structural antigen may be immunogenic in a mammal. The mammal may be human. The immune response may be a protective immune response. The Zika virus structural antigen may be capable of activating T-cell and antibody mediated immunity in a subject. The protein may be capable of activating T-cell mediated immunity in a subject. The protein may be capable of activating antibody-mediated immunity in a subject.

The Zika viral vector vaccine may be used as a vaccine in combination with another therapeutically or prophylactically active ingredient. The Zika viral vector vaccine may be used as a vaccine in combination with an adjuvant.

The Zika viral vector vaccine may be provided in a pharmaceutically acceptable carrier.

According to another aspect of the invention there is provided a nucleic acid encoding the Zika viral vector vaccine according to the first or second aspect of the invention, or parts thereof.

The nucleic acid may be a plasmid vector for vaccination.

According to another aspect of the invention there is provided a composition comprising the nucleic acid according to the invention or the viral vector according to the invention.

The composition may be immunogenic, for example in a mammal, such as a human.

The composition may comprise a pharmaceutically acceptable carrier. The composition may be a pharmaceutical composition comprising a pharmaceutically acceptable carrier. The composition may be for use in the prophylaxis or treatment of Zika viral infection.

According to another aspect of the invention there is provided a method of treatment or prophylaxis of Zika viral infection comprising the administration of:
the nucleic acid according to the invention;
the composition according to the invention or
the viral vector according to the invention.

The method of treatment or prophylaxis of Zika viral infection may be a method of vaccination.

According to another aspect of the invention there is provided an agent for use in the prophylaxis or treatment of Zika viral infection, the agent comprising or consisting of:
the nucleic acid according to the invention;
the composition according to the invention or
the viral vector according to the invention.

According to another aspect of the invention there is provided the composition according to the invention; the nucleic acid according to the invention; or the viral vector according to the invention; for use in, or as, a vaccine.

The Zika viral vector vaccine may be used in a prime and/or boost vaccine formulation. The vaccine may be a prime vaccine. The vaccine may be a boost vaccine. Where a boost vaccine is provided following a prime vaccine, the protein may be different. The prime-boost may comprise an initial vaccination with an adenovirus, followed by a MVA expressing the same antigen according to the invention.

According to another aspect of the invention, there is provided a prime boost vaccination kit comprising
a prime vaccination according to the invention;
a boost vaccination according to the invention.

The prime and boost vaccinations may be different. The prime and boost vaccination may differ in the protein sequence. The prime and boost vaccination may comprise different viral vectors.

### Combination Zika and Chikungunya Vaccine

The sudden presence of Zika and Chikungunya in the same geographical regions have overwhelmed health systems that were already challenged by Dengue, thus increasing the failure to provide treatment and preventive measures to their populations during the outbreak, while posing new challenges for treatment of both Zika and Chikungunya due to the long-term sequelae of more than 6 years for these diseases. A major breakthrough is required to provide governments with tools to simultaneously fight these highly prevalent arbovirus diseases and a multivalent vaccine able to protect against both Zika and Chikungunya, which would be an ideal preventive solution.

According to another aspect of the invention there is provided the Zika viral vector vaccine according to the invention herein in combination with a Chikungunya vaccine.

Advantageously, the present invention provides that both vaccines can be injected as a bivalent formulation without compromising immunogenicity. Surprisingly, it was observed that a mixture in the same syringe of the two vaccines or a co-vaccination in different legs induced similar antibody responses to those induced individually by the vaccines over 20 weeks after a single vaccination or one week post MVA boost.

In one embodiment, the Zika viral vector vaccine according to the invention herein is co-formulated in the same composition with the Chikungunya vaccine.

Therefore, according to another aspect of the invention there is provided a composition comprising the Zika viral vector vaccine according to the invention herein and a Chikungunya vaccine.

According to another aspect of the invention there is provided the Zika viral vector vaccine according to the invention herein for use in combination with a Chikungunya vaccine.

The use may be for treatment or prevention of Zika viral infection and/or Chikungunya viral infection. In one embodiment, the use may be for treatment or prevention of Zika viral infection and Chikungunya viral infection.

The use may be in a combined formulation. In another embodiment, the use may be concurrent or sequential administration (e.g. formulated separately, but administered together).

According to another aspect of the invention there is provided a method of vaccination for prevention or treatment of Zika viral infection and/or Chikungunya viral infection, the method comprising the administration of the Zika viral vector vaccine according to the invention herein and a Chikungunya vaccine.

The administration may be the administration of a combined formulation. In another embodiment, the administration may be concurrent or sequential administration (e.g. formulated separately, but administered together) of the Zika viral vector vaccine according to the invention herein and a Chikungunya vaccine.

In one embodiment, the Chikungunya vaccine is a Chikungunya viral vector vaccine. The Chikungunya viral vector vaccine may comprise nucleic acid encoding one or more Chikungunya structural antigens. The Chikungunya viral vector vaccine may comprise nucleic acid encoding one or more Chikungunya structural antigens with or without the capsid. The Chikungunya viral vector vaccine may comprise nucleic acid comprising the sequence of SEQ ID NO: 14 or SEQ ID NO: 16, or variants thereof. In another embodiment, the Chikungunya viral vector vaccine comprises nucleic acid encoding polypeptides comprising the sequence of SEQ ID NO: 15 or SEQ ID NO: 17, or variants thereof.

The skilled person will understand that some modifications or variants of the Chikungunya vaccine sequences may provide the same or substantially similar immunogenic function. Modifications may comprise nucleotide additions, substitutions, or deletions. In one embodiment, the modification may encode for no more than 60, 30, 20, 15, 10, 9, 8, 6, 5, 4, 3, 2, 1 nucleotide additions, substitutions, or deletions. The nucleotide additions, substitutions, or deletions may involve consecutive nucleotides, multiple groups of nucleotides, one or more codons, or non-consecutive nucleotides, or combinations thereof. Modifications may comprise conservative substitutions of nucleotides using codon redundancy to encode the same Chikungunya vaccine protein sequences described herein.

Variants of SEQ ID NO: 14 or SEQ ID NO: 16 may comprise or consist of a sequence having at least 80% identity with SEQ ID NO: 14 or SEQ ID NO: 16. Variants of SEQ ID NO: 14 or SEQ ID NO: 16 may comprise or consist of a sequence having at least 85% identity with SEQ ID NO: 14 or SEQ ID NO: 16. Variants of SEQ ID NO: 14 or SEQ ID NO: 16 may comprise or consist of a sequence having at least 90% identity with SEQ ID NO: 14 or SEQ ID NO: 16. Variants of SEQ ID NO: 14 or SEQ ID NO: 16 may comprise or consist of a sequence having at least 95% identity with SEQ ID NO: 14 or SEQ ID NO: 16. Variants of SEQ ID NO: 14 or SEQ ID NO: 16 may comprise or consist of a sequence having at least 98% identity with SEQ ID NO: 14 or SEQ ID NO: 16. Variants of SEQ ID NO: 14 or SEQ ID NO: 16 may comprise or consist of a sequence having at least 99% identity with SEQ ID NO: 14 or SEQ ID NO: 16.

Variants may comprise amino acid modification. Amino acid modifications may comprise amino acid residue additions, substitutions, or deletions. In one embodiment, the modification may encode for no more than 20, 15, 10, 9, 8, 6, 5, 4, 3, 2, or 1 amino acid residue additions, substitutions, or deletions. The amino acid residue additions, substitutions, or deletions may involve consecutive amino acid residues, multiple groups of amino acid residues, or non-consecutive amino acid residues, or combinations thereof. Nucleic acid variants may comprise nucleotide modifications. Nucleotide modifications may comprise nucleotide additions, substitutions, or deletions. In one embodiment, the modification may encode for no more than 60, 50, 40, 30, 20, 15, 10, 9, 8, 6, 5, 4, 3, 2, or 1 nucleotide additions, substitutions, or deletions. The nucleotide additions, substitutions, or deletions may involve consecutive nucleotides, multiple groups of nucleotides, codons, or non-consecutive nucleotides, or combinations thereof.

Variants of SEQ ID NO: 15 or SEQ ID NO: 17 may comprise or consist of a sequence having at least 80% identity with SEQ ID NO: 15 or SEQ ID NO: 17. Variants of SEQ ID NO: 15 or SEQ ID NO: 17 may comprise or consist of a sequence having at least 85% identity with SEQ ID NO: 15 or SEQ ID NO: 17. Variants of SEQ ID NO: 15 or SEQ ID NO: 17 may comprise or consist of a sequence having at least 90% identity with SEQ ID NO: 15 or SEQ ID NO: 17. Variants of SEQ ID NO: 15 or SEQ ID NO: 17 may comprise or consist of a sequence having at least 95% identity with SEQ ID NO: 15 or SEQ ID NO: 17. Variants of SEQ ID NO: 15 or SEQ ID NO: 17 may comprise or consist of a sequence having at least 98% identity with SEQ ID NO: 15 or SEQ ID NO: 17. Variants of SEQ ID NO: 15 or SEQ ID NO: 17 may comprise or consist of a sequence having at least 99% identity with SEQ ID NO: 15 or SEQ ID NO: 17.

The skilled person will recognise that any immunogenic or effective Chikungunya vaccine may be used in combination with the Zika viral vectored vaccine of the present invention. The first CHIKV vaccines described were formalin-inactivated vaccines. Interestingly, formalin-inactivated CHIKV prepared from chicken embryos did not induce potent, protective immune responses (White et al. Appl Microbiol. 1972 May; 23(5):951-2). A live-attenuated CHIKV vaccine candidate (termed strain 181/clone25) was developed at the US Army Medical Research Institute of Infectious Diseases (USAMRIID), although in produce good antibody responses, there is evidence of reactogenicity (small signs of arthralgias in vaccine) and genetic instability (Levitt et al. Vaccine. 1986 Sep; 4(3): 157-62.). A DNA vaccine comprising E1, E2, E3 protected mice when it was injected in at least 3 separate doses. However, Capsid DNA vaccine did not protect (Muthumani et al. Vaccine. 2008 Sep. 19; 26(40): 5128-34. doi: 10.1016/j.vaccine.2008.03.060. Epub 2008 Apr. 14). A VLP-based vaccine expressing the CHIKV envelope proteins produced high-titered neutralizing antibodies in monkeys after three doses, and protected them against viremia after challenge (Akahata et al. Nat Med. 2010 Mar; 16(3): 334-8. doi: 10.1038/nm.2105. Epub 2010 Jan. 28). Because some CHIKV vaccines candidates need multiple immunisations, a better and cheaper alternative to produce vaccine carrier is needed. In this regard, viral vectored vaccines carrying CHIKV are being under development. Wang et al. (Vaccine. 2011 Mar. 24; 29(15): 2803-2809) developed an Adenovirus based Vaccine, where a single immunisation induced high titres of neutralising anti-chikungunya virus antibodies. MVA has also been developed to express CHIKV E3-E2 proteins, which generate protective immune responses (Weger-Lucarelli et al. PLoS Negl Trop Dis. 2014 Jul; 8(7): e2970.). Anyone of these Chikungunya vaccines may be used as the Chikungunya vaccine component of the present invention.

The Chikungunya viral vector may comprise an adenovirus, such as a simian adenovirus. The viral vector may comprise an adenovirus when used in a prime vaccine of a prime boost regime. The viral vector may comprise ChAdOx1 (a group E simian adenovirus, like the AdCh63 vector used safely in malaria trials). The viral vector may comprise AdCh63. The viral vector may comprise AdC3 or AdH6. The viral vector may be a human serotype. The viral vector may comprise Modified Vaccinia Ankara (MVA). The viral vector may comprise MVA when used as a vaccine boost in a prime boost regime. The viral vector may comprise Adeno-associated virus (AAV) or lentivirus. The viral vector may be an attenuated viral vector. The protein encoding sequence of the Chikungunya vaccine may be cloned into any suitable viral vector that is known to elicit good immune response.

The skilled person will be familiar with vaccine administration routes and doses. For example the administration may be sub-cutaneous, intra-muscular, or intravenous.

The Zika viral vector according to the invention may not encode the full wild-type sequence of Zika virus (i.e. the sequence is partial/incomplete or modified). The Zika viral vector according to the invention may encode a fusion protein. The Zika viral vector according to the invention may comprise a synthetic sequence (i.e. not seen in nature).

The term “immunogenic”, when applied to the protein or composition of the present invention means capable of eliciting an immune response in a human or animal body. The immune response may be protective.

The term “protective” means prevention of a disease, a reduced risk of disease infection, transmission and/or progression, reduced severity of disease, a cure of a condition or disease, an alleviation of symptoms, or a reduction in severity of a disease or disease symptoms.

The term “prophylaxis” means prevention of or protective treatment for a disease. The prophylaxis may include a reduced risk of disease infection, transmission and/or progression, or reduced severity of disease.

The term “treatment”, means a cure of a condition or disease, an alleviation of symptoms, or a reduction in severity of a disease or disease symptoms.

Reference to sequence “identity” used herein may refer to the percentage identity between two aligned sequences using standard NCBI BLASTp or BLASTn parameters as appropriate (http://blast.ncbi.nlm.nih.gov).

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

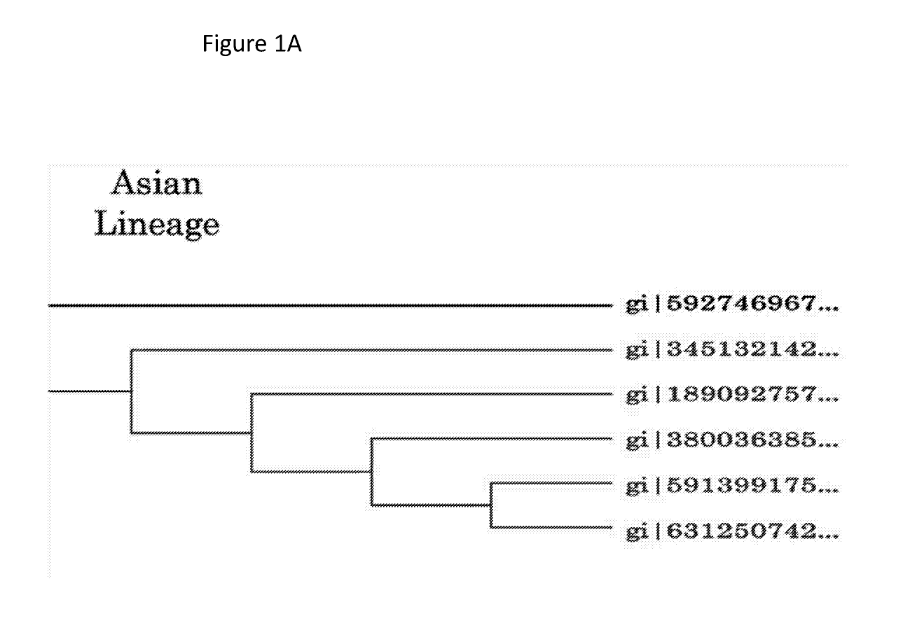

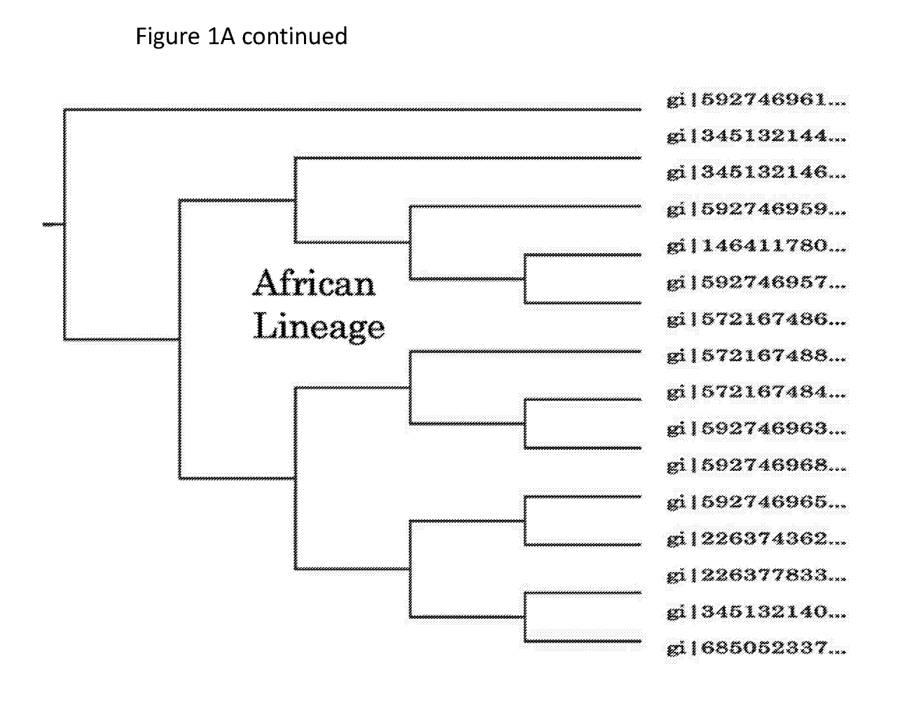

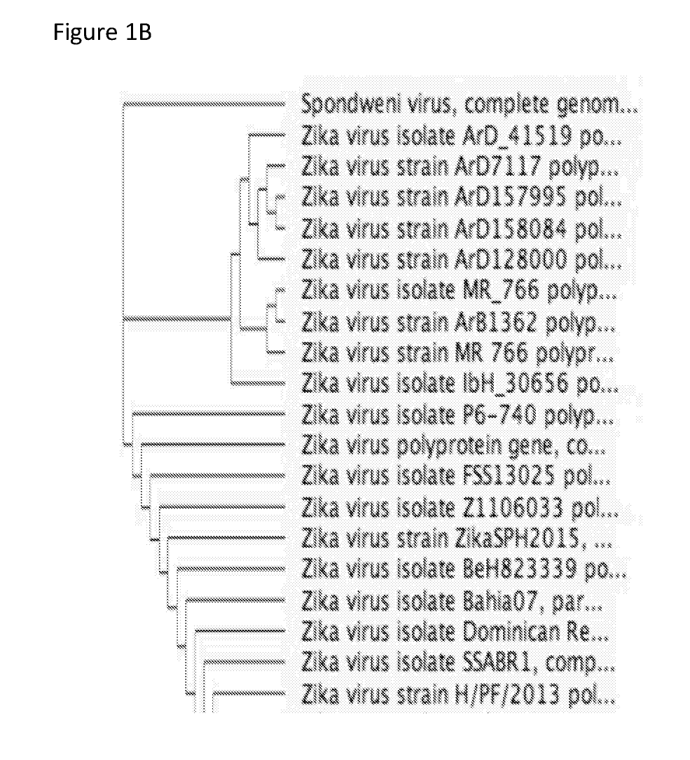

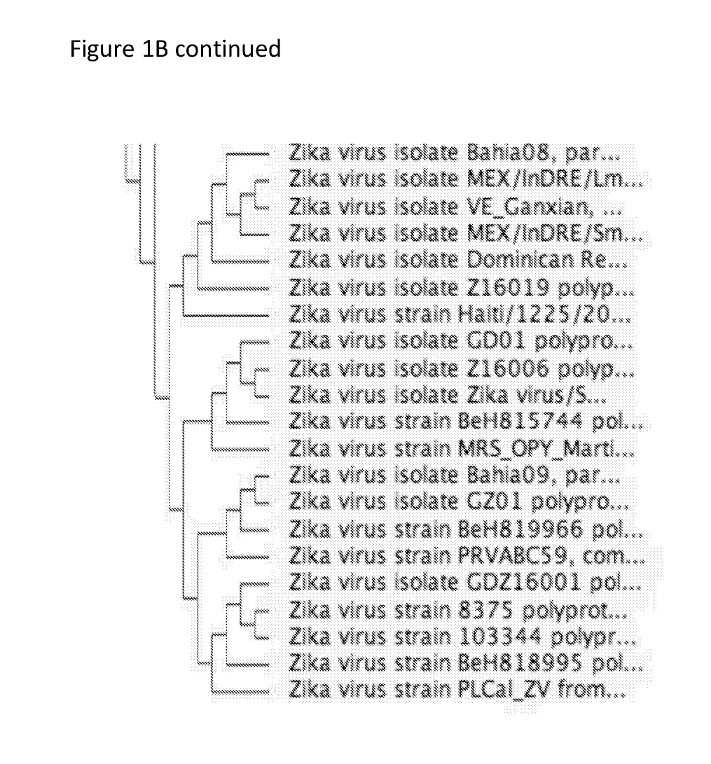

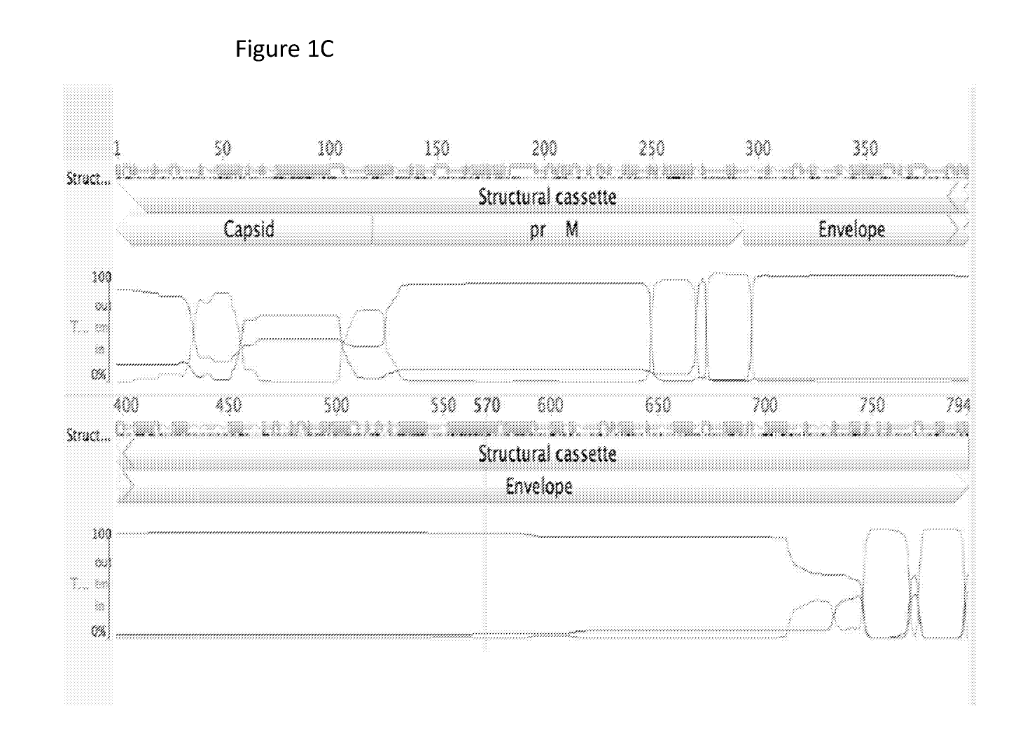

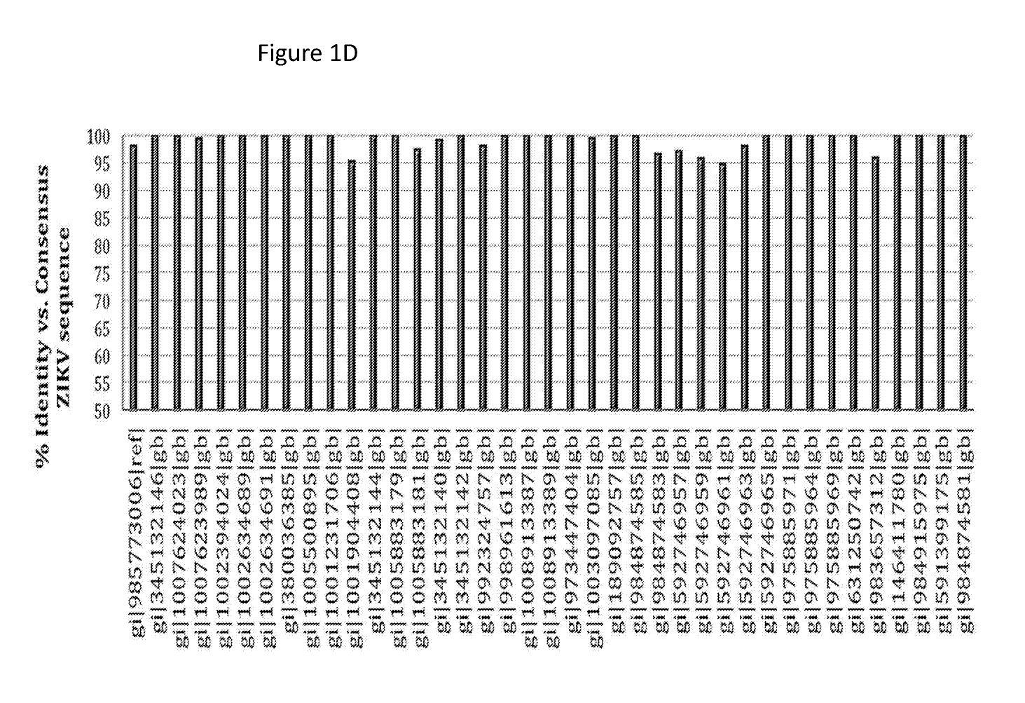

FIG. 1. Construction of a Zika Virus consensus sequence. (A) Available sequences from Zika Virus were gathered; sequences were curated and only full genome sequences were used for further analysis. Sequences belonging to both African and Asian lineages were identified, sorted by host species and geographical locations. Special attention was given to human isolates (African, Asian and Imported cases). Initial phylogenetic tree was produced with the sequences available as in 27/Nov/2015/ (A, left). As in April 2016 an updated phylogenetic tree was also produced, and the amount of available genetic sequences increased by 6-fold (A, right). Nucleotide and protein alignment was performed; by the 10th of December 2015, Zika Virus sequences available in the gene-bank were not annotated. Annotation was performed by sequence similarity with Dengue Virus and Yellow Fever Virus genomes/proteins. Two types of consensus sequences were built: a consensus that covered only the Asian lineage and a consensus sequence covering both Asian and African linages. We have produced for both a whole genome consensus sequence that covered structural (Capsid, prM, Envelope) and non-structural (NS1, NS2, NS3, NS4 and NS5). For the Zika virus structural genes, we focused in the prM and Envelope consensus sequence that was built from the Zika Asian Lineage as this lineage is the one that has spread across the Americas (C). Conservation between African and Asian sequences was about 92% (data analysis not shown). Consensus sequences were as close as 95-100% similarity between both lineages when compared to all available genome sequences as in April 2016 (D). Therefore, a prM and Envelope structural DNA cassette was requested to Geneart (Thermofisher) for synthesis. The Zika prM-Envelope sequence was codon-optimised and designed to allow sub-cloning to pMono and MVA plasmids, recombination with ChAdOx1 (adenovirus), as well as restriction sites used for cloning to Phlsec (protein production)

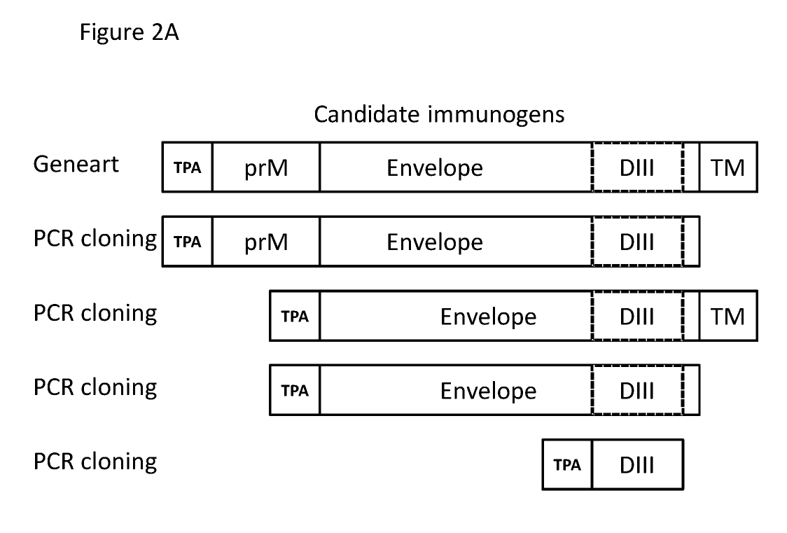

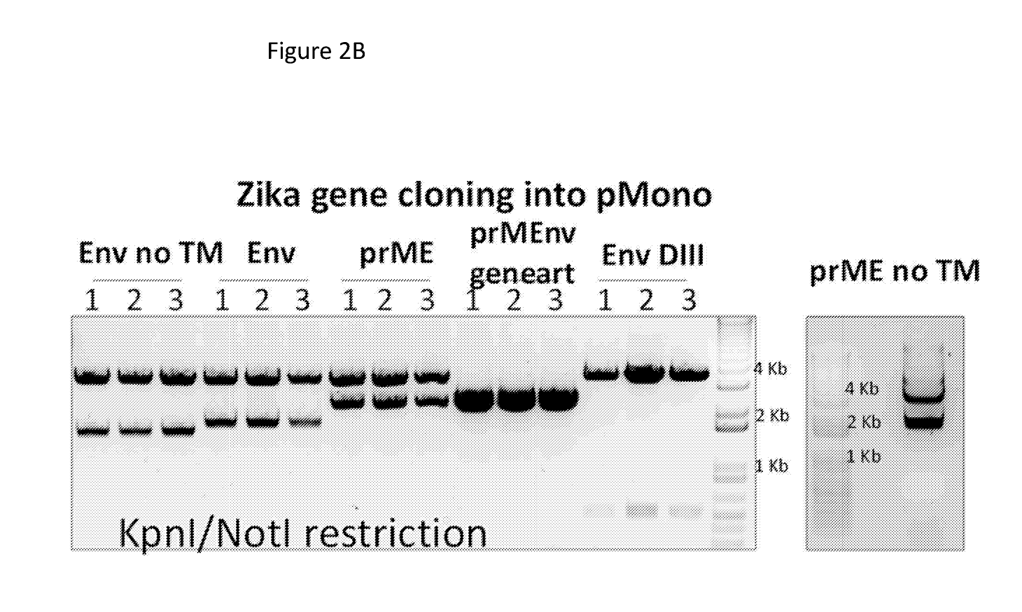

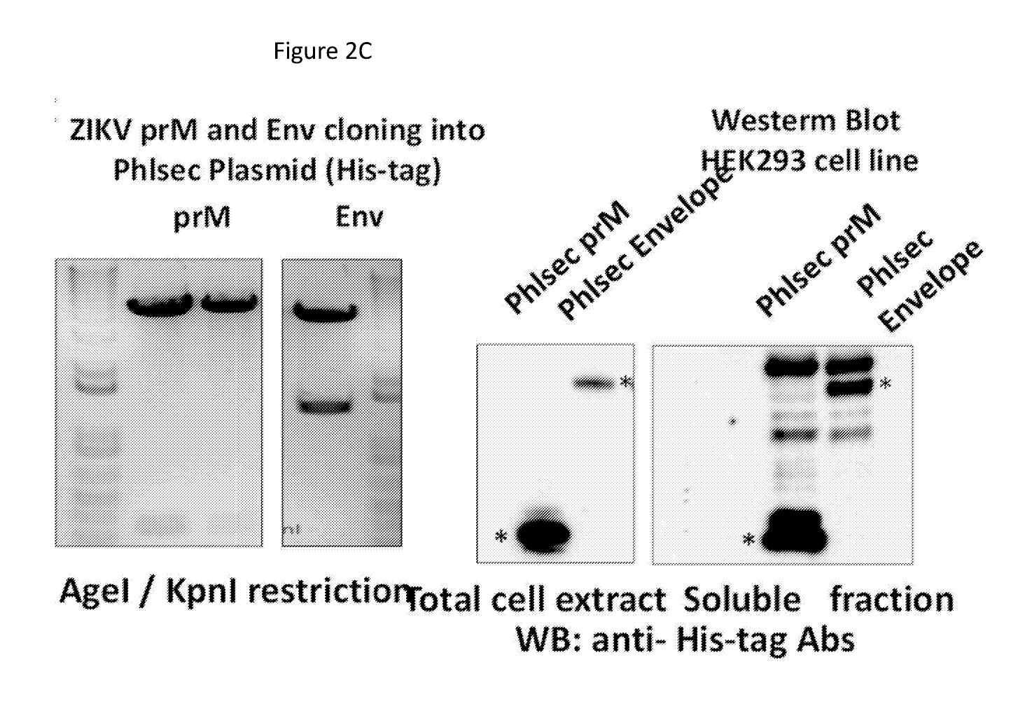

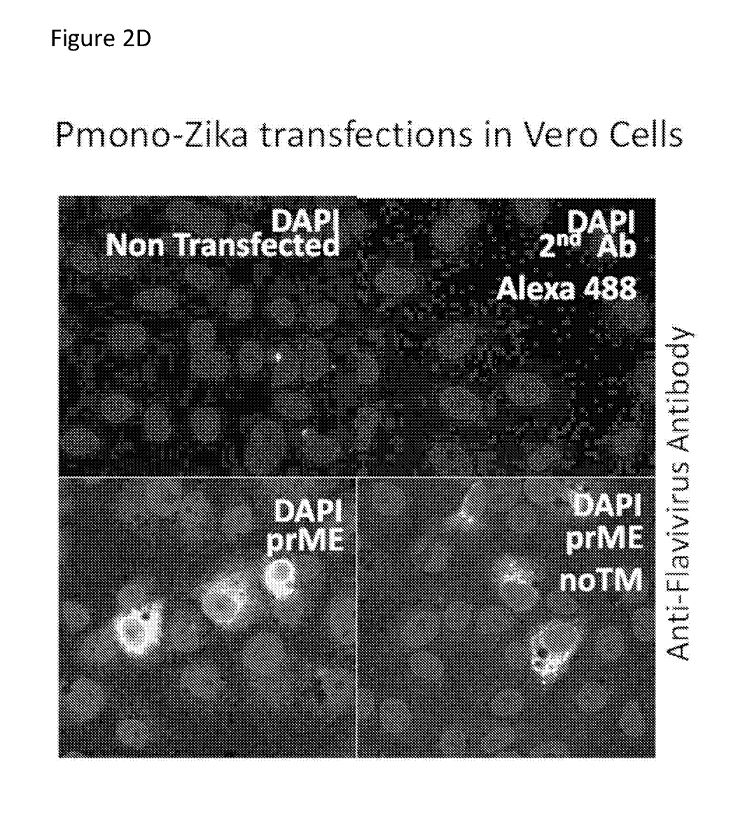

FIG. 2. ZIKV versions produced from consensus transgene. (A) A synthetic genetic consensus sequence encoding the Zika virus prM and Envelope (Env) protein was produced (geneart™) as in FIG. 1 (construct 1). Polymerase Chain Reaction (PCR)-cloning was performed using construct 1 as a template and using specific primers to produce the following DNA constructs: a prM and Env lacking the transmembrane domain (TMD) (construct 2); a full Env lacking prM (construct 3); an Env lacking prM and TMD (construct 4) and an Env-domain III only (DIII) (construct 5). (B, left and right) All DNA constructs were sub-cloned by restriction and DNA-ligation into an expression plasmid under the CMV promoter activity, denominated pMono. Restriction analysis released specific DNA band-sizes, corresponding to all constructs and empty pmono plasmids. Alternatively, all DNA pmono plasmids were confirmed by DNA sequencing. (C) As a proof of plasmid expression, Vero cells were transfected with construct 1 and construct 2, respectively. After transfection, cells were subjected to immunofluorescence (IF) against a mouse anti-Flavivirus Envelope antibody and later stained with an Alexa 488 conjugated goat anti-mouse Ig antibody. Green cells showed that those plasmids are able to induce expression of Zika Envelope. (D) prM and Env DNA was sub-cloned into a Phlsec His-tagged plasmid for protein expression in HEK293 cells; restriction analysis (left) showed specific DNA sizes for both prM and Env. HEK293 cells were transfected with Phlsec-prM and Phlsec-Env and a western blot was performed using an HRP-conjugated anti-mouse His tag antibody (right panel). * shows specific His-tag recognition in both total cell and soluble fractions.

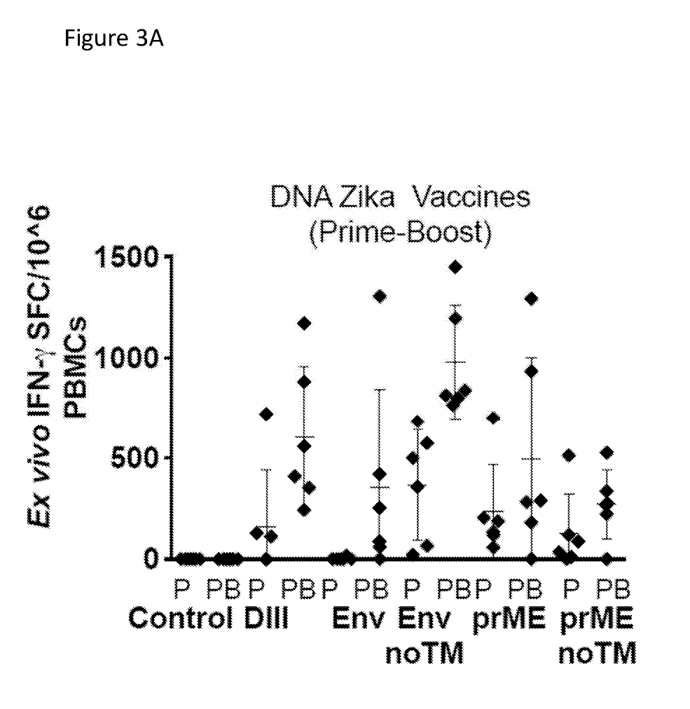

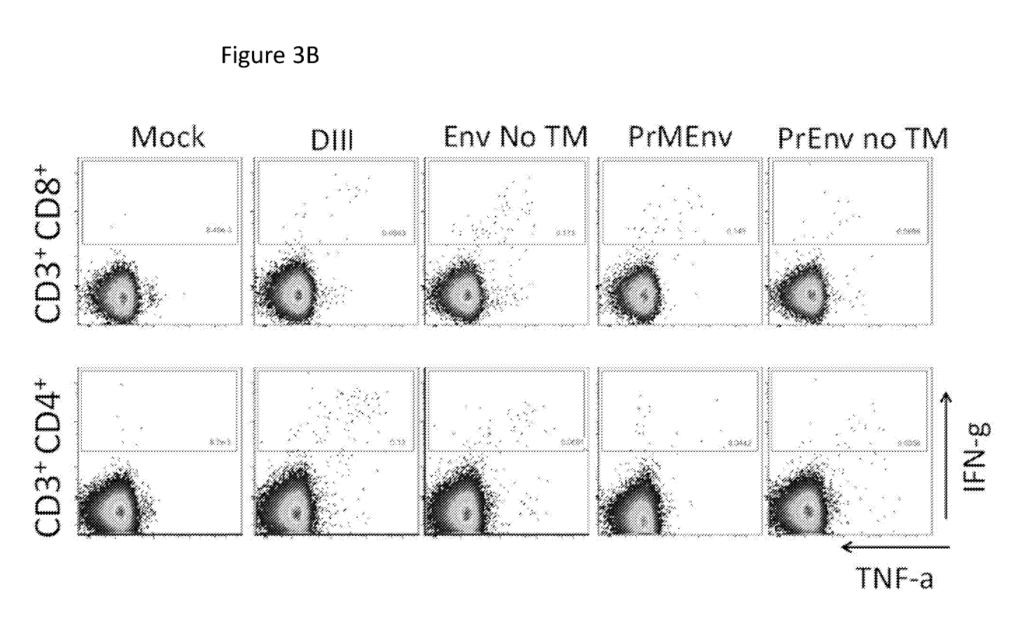

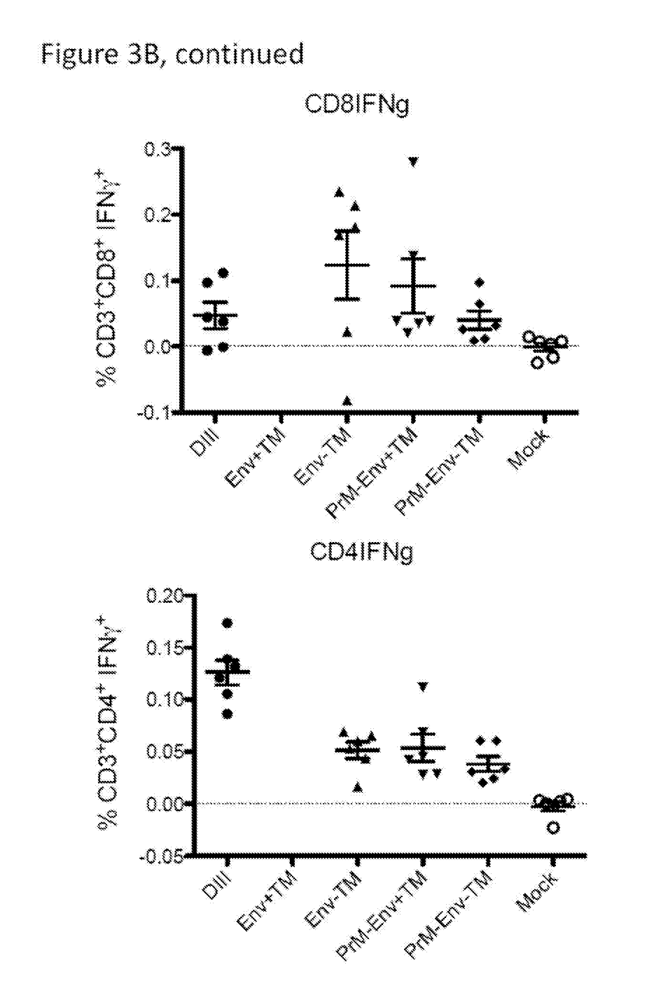

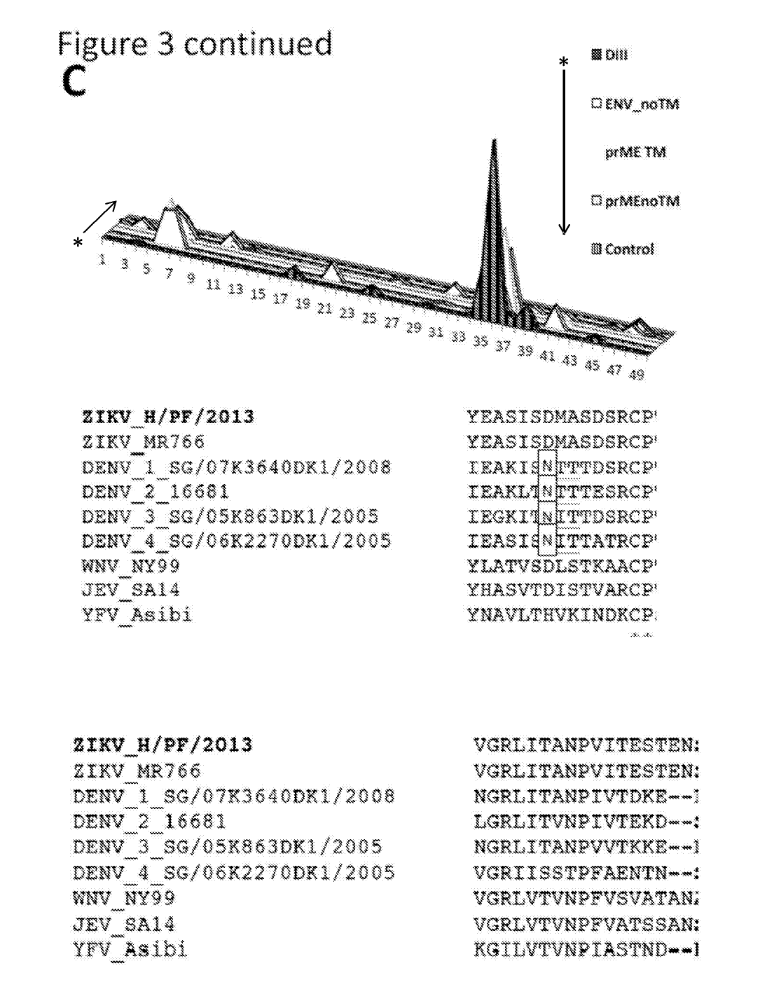

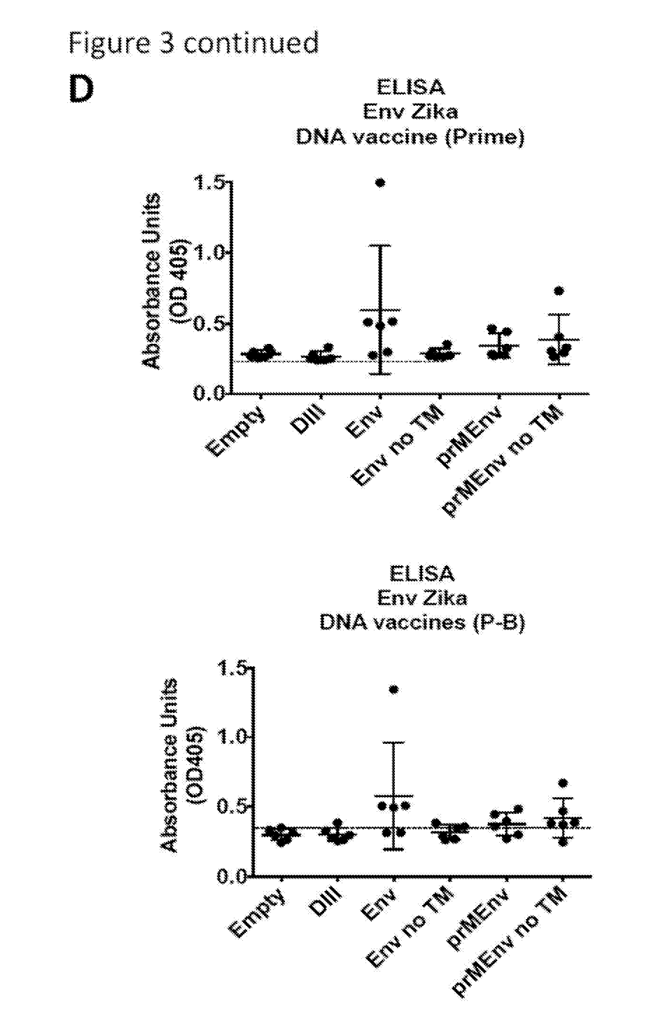

FIG. 3. Assessment of mice immunogenicity after Zika-DNA vaccination. Groups of BalbC mice were immunised (prime) with the Zika DNA vaccines shown in FIG. 2A and 2B. Two weeks after the prime, mice were bled to isolate peripheral blood mononuclear cells (PBMCs) and sera. Same groups were subjected to a second immunisation two weeks after the prime (boost). PBMCs and sera were also recovered two weeks after boost. For T-cell responses, a pool of 20mer peptides (10aa overlap) comprising the full Env was prepared. (A) Elispot analysis showed that all DNA vaccinations elicited T-cell responses. Importantly, responses for INF-g varied within groups in both prime (grey) or boost (red) regimes. T-cell responses were modulated based on having or not the prM and/or the TMD. For example, DNA vaccine encoding the Envelope with no TMD gave the highest responses. Full version of ENV elicited 3-fold down INFg producing PBMCs in comparison with Env no TMD. Combination of prM and TMD also impacted the breath of T-cell INFg responses in all groups. (B) Intracellular Cytokine Staining (ICS) and flow cytometry analysis was performed on the Prime-Boost mice groups. Based on the analysis it can be concluded that all vaccines elicited both CD8 (top panels) and CD4 (bottom panels) T-Cells. Further analysis on those samples confirmed that the DNA vaccine carrying the Envelope with no TMD elicited the highest CD8 T-cell responses shown in the ELispot data (FIG. 2A), whereas the DNA vaccine carrying the Env DIII only elicited the highest CD4 T-Cells responses. Again, modulation of both CD8 and CD4 T-Cell response were achieved by absence or presence of TMD and/or prM. Note Env with TMD sample was lost during processing. (C) BalbC spleenocytes from mice vaccinated in a Prime-Boost DNA vaccine regime were subjected to epitope T-Cell mapping by stimulating T-Cells with every single 20-mer peptide spanning the whole Zika virus envelope. 50 peptides where used to screen and identify the most immunogenic peptides. We have identified two immunodominant peptides: peptide number 7 (YEASISDMASDSRCP) and peptide number 36 (VGRLITANPVITESTEN). Further conservation analysis (C, right) shows the degree of peptide homology between other flaviviruses such as Dengue (Alignments and figures modified from Science 22 Apr. 2016: Vol. 352, Issue 6284, pp. 467-470). (D) Enzyme Linked Immuno-Sorbent Assay (ELISA) revealed that DNA vaccines were able to produce antibodies against the Zika Envelope protein as measured by OD405 colorimetric levels. However, Prime-Boost regime (right) seemed to maintain almost the same antibody levels of that reached by a single DNA vaccination (left). DNA vaccine carrying the full Envelope protein elicited the most detectable antibodies in comparison with a DNA vaccine control. Other vaccines elicited very modest responses right above the background (dotted line representing the average of control OD405 background plus 2 times their SD).

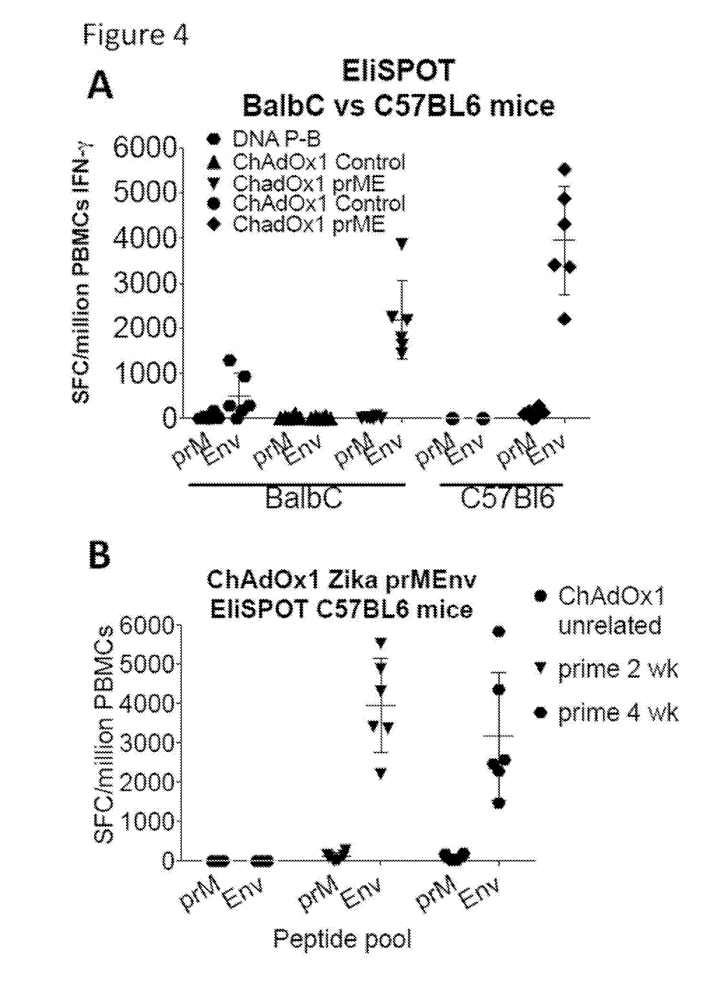

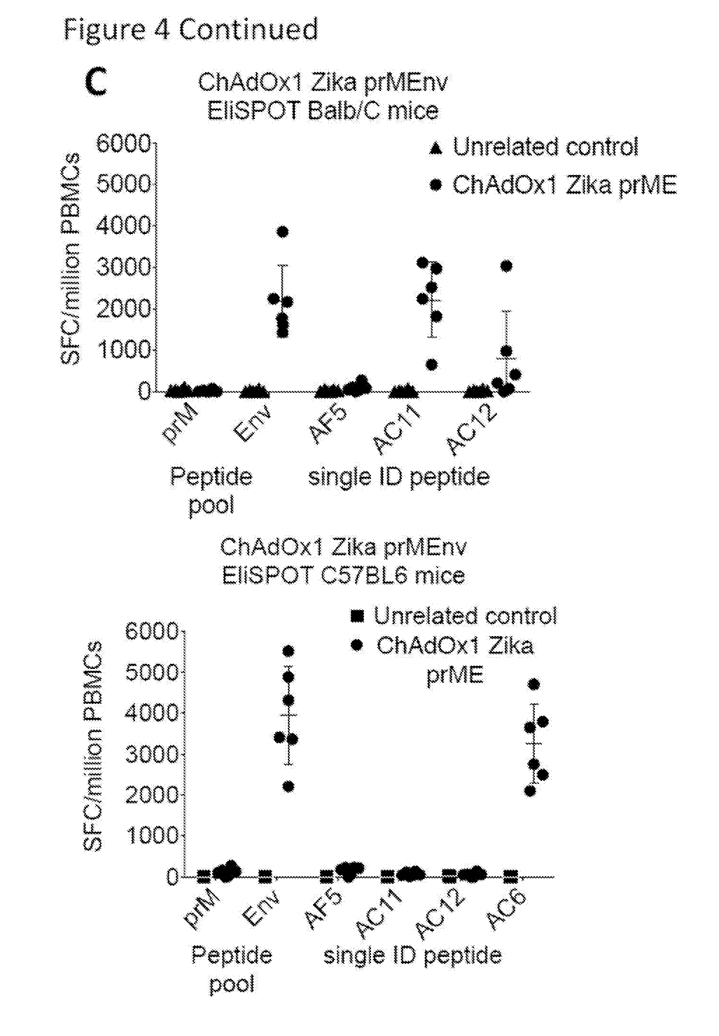

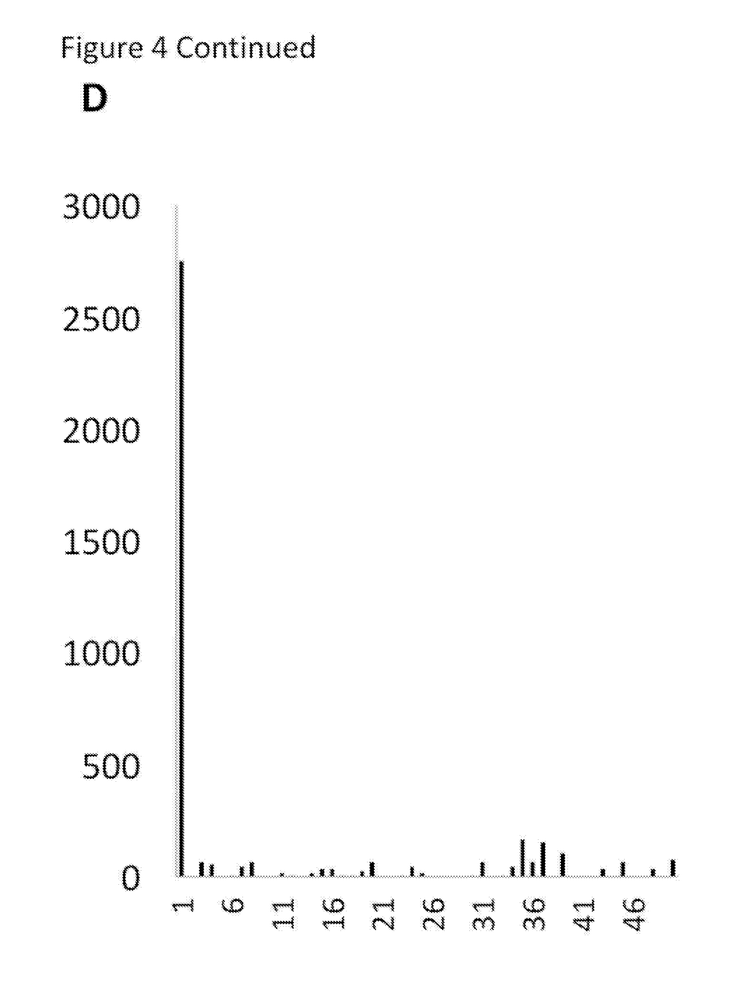

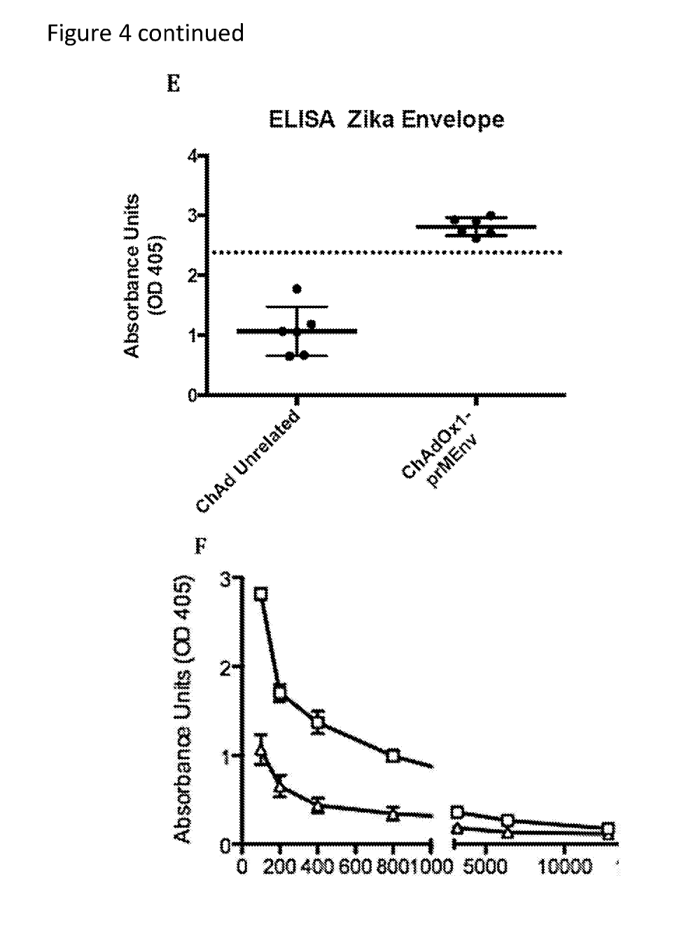

FIG. 4. Mice immunogenicity after a single dose of the Adenoviral-vectored ChAdOx1-Zika vaccine. (A) Naïve BalbC and C57BL6 mice strains were immunised with 10e8 IU of a ChAdOx1-Zika vaccine carrying the prM and full Envelope genes. ELispot assay was performed 2 weeks after prime, using Zika Envelope peptides. Higher INFg responses were found in C57BL6 (black dots) than BalbC mice (green), being both responses abundantly higher (4 an 8-fold increase) in comparison with the responses elicited in a prime-boost DNA vaccination regime that carries the same antigens (red dots). Unrelated ChAdOx1 was used as a control (purple and blue dots). Modest INFg responses against prM were detected. (B) IFNg responses from C57BL6 mice were followed for 2 weeks (black dots) and 4 weeks (green dots) after prime with ChAdOx1-Zika immunisation. T-cell responses maintained the immunogenic profile seen in standard adenoviral vectored vaccines. (C) Immunodominant peptides detected in BalbC mice DNA vaccination as in FIG. 3 were confirmed in mice vaccinated with ChAdOx1-Zika (right panel). For C57BL6 mice, those peptides were not immunogenic but a single peptide ID:AC6 (left panel). The peptide corresponded to the starting N-terminal region of Zlka envelope (IRCIGVSNRDFVEGMSGGTW) and that share low homology to other known flaviviruses (D top and bottom figure). (Alignments and figures modified from Science 22 Apr. 2016: Vol. 352, Issue 6284, pp. 467-470) (E) Enzyme Linked Immuno-Sorbent Assay (ELISA) revealed that ChAdOx1-Zika vaccine carrying the prM and full Envelope genes were able to produce antibodies against the Zika Envelope protein as measured by OD405 colorimetric levels in comparison with a unrelated ChAdOX1 vaccine control. (Background is represented as a dotted line, which is the average of control OD405, plus 3 times their SD). (F) Further dilution of sera from vaccinated mice were plotted against OD405 showing the increase of OD405 in ChAdOx1-Zika vaccinated mice (squares) in comparison with control (triangles).

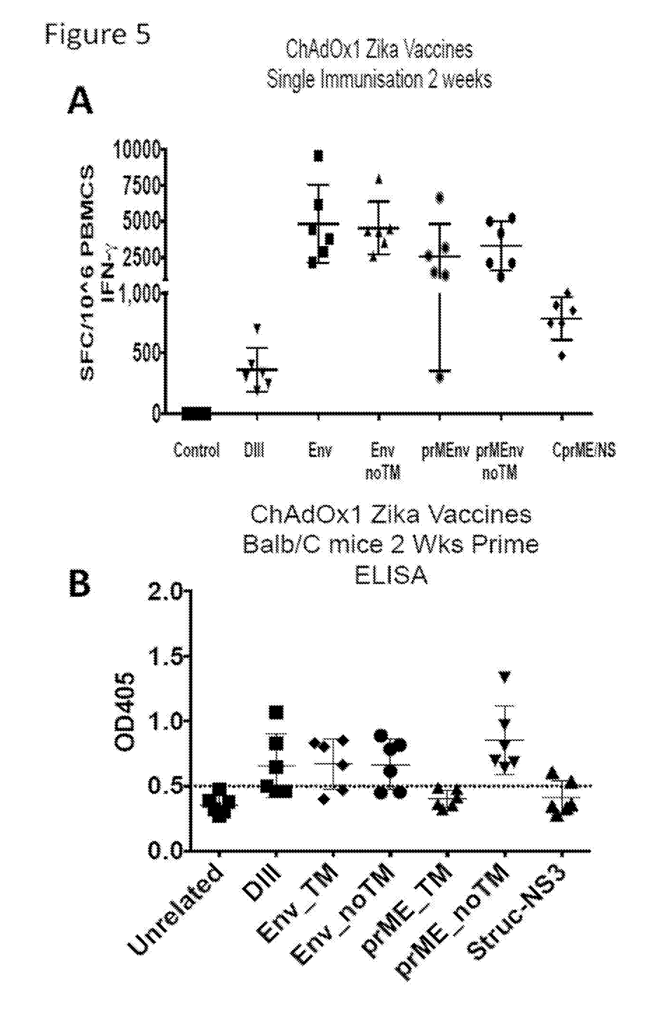

FIG. 5 shows (A) Cellular immune responses, which were quantified in BALB/c mice following an immunisation with the ChAdOx1-Zika vaccines. 14 days post-vaccination, peripheral blood mononuclear cells (PBMCs) were obtained by tail bleeding. Cells were resuspended using EDTA anticoagulant. PBMCs were further purified by eliminating or lysing red blood cells and were suspended in DMEM media, plated in ELISpot plates with PDVF membranes. PBMCs were incubated during 18 hours in presence of peptide pools spanning the whole structural region of the zika virus. Peptide pools consisted on 20-mers overlapping by 10 and were used at a final concentration of bug per peptide. Results are expressed as spot-forming colonies per million PBMCs and the responses indicated are ex vivo, which means no further incubation to expand cells and increase responses was made, and all the frequencies reported are from cells tested immediately after bleeding.

DIII resulted in the lowest T cell responses and this perhaps indicates that these are CD4s, which would be confirmed by flow cytometry. The rest of the constructs induced robust T cell responses in averages between 3,000 and 5,000 SFU/million PBMCs.

(B) FIG. 5B indicates antibody responses elicited after immunisation with the various versions of ChAdOx1-Zika vaccines, as indicated in the figure. Immunisations were made as described in A.

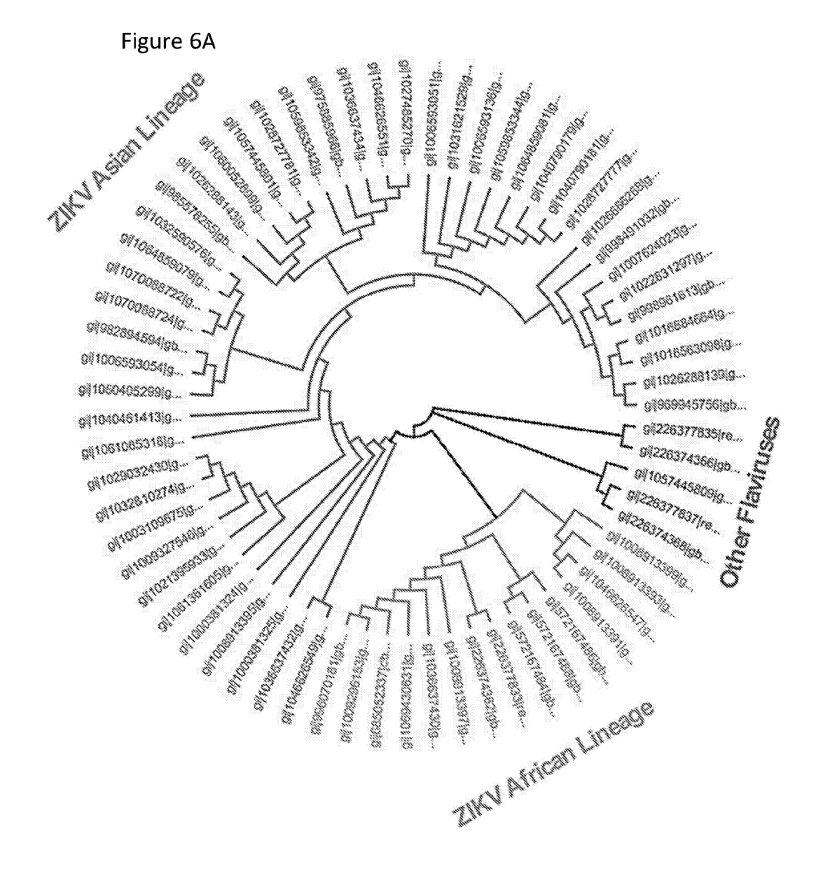

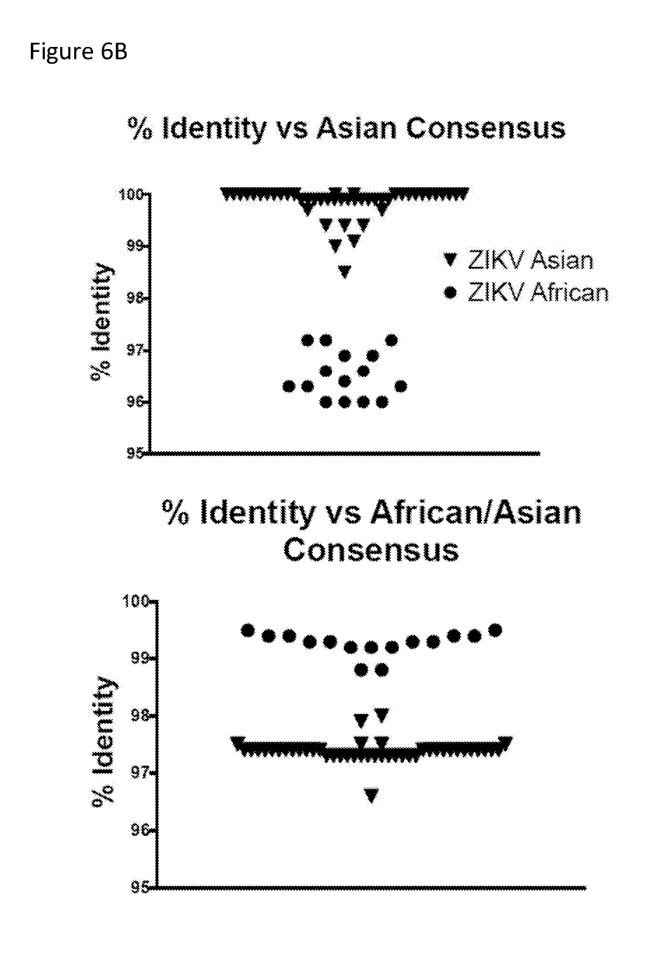

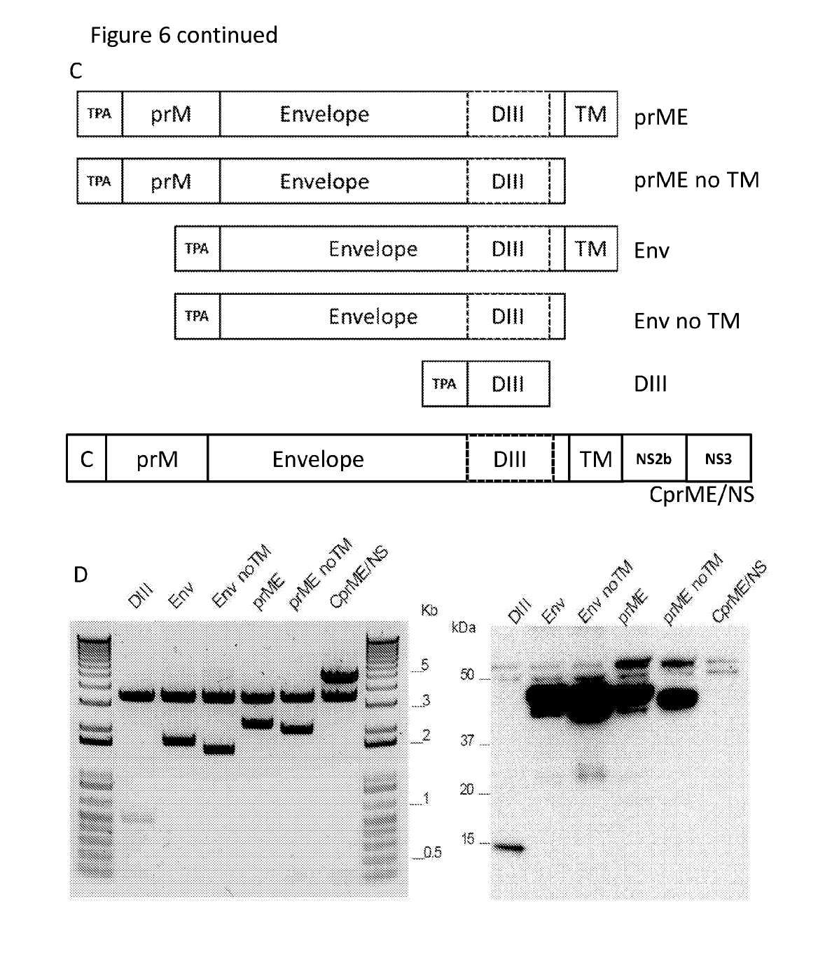

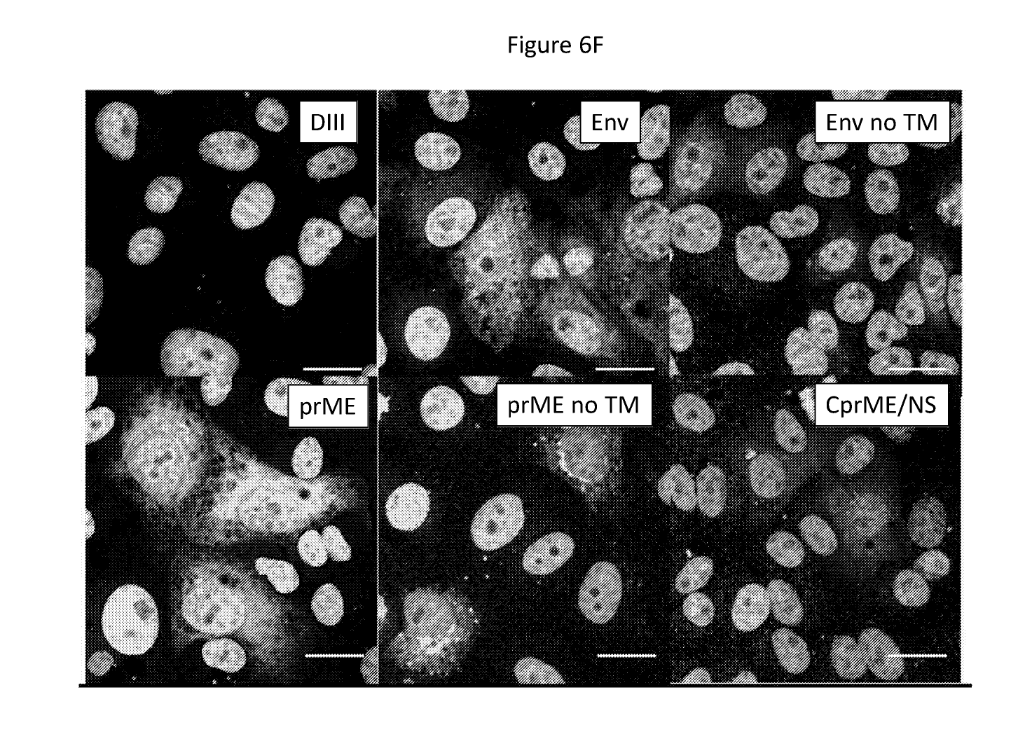

FIG. 6. Zika vaccine design. (A) A phylogenetic tree for ZIKV genomes up to October 2016; blue, red and green labels represent the Asian and African lineages of ZIKV and other Flaviviruses (such as DENV), respectively. (B) Conservation homology of Asian (top) and African/Asian (bottom) consensus sequences versus all genomic sequences depicted in A; circle represents the ZIKV-BR strain used for the challenge experiment. (C) Schematic representation of the Zika immunogen versions used in this study; cross-hatch block represents the TPA leading sequence. (D) Restriction enzyme analysis of the plasmid DNA vaccines constructed, a 3.3 Kb band size represents the Pmono plasmid back bone. (E) HEK293 expression of the plasmid DNA encoding the Zika immunogens using a generated anti-ZIKV Envelope antibody. (F) Immunofluorescence analysis of Vero cells transfected with plasmid DNA encoding the ZIKV immunogens as depicted in D; using a commercial anti-flavivirus antibody.

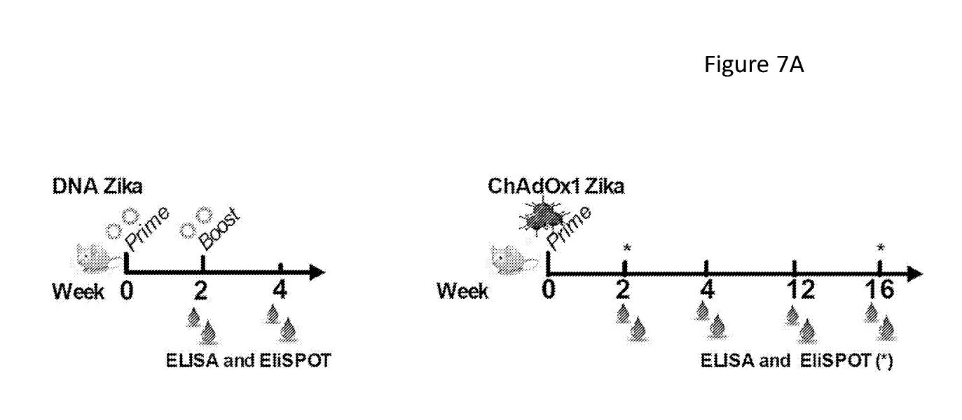

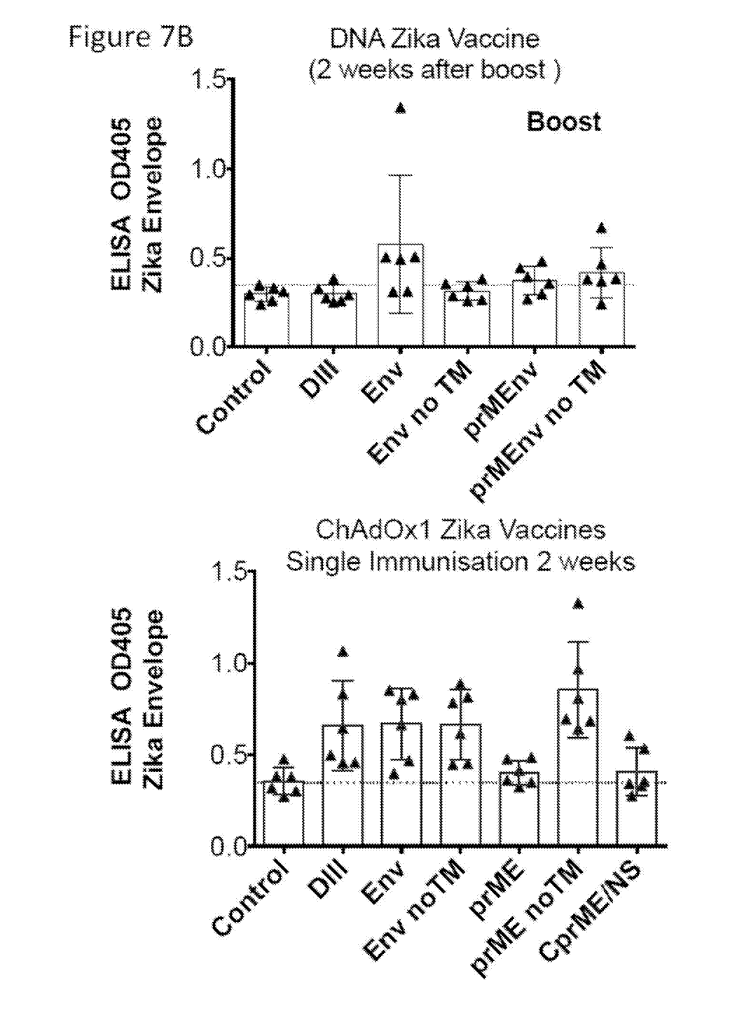

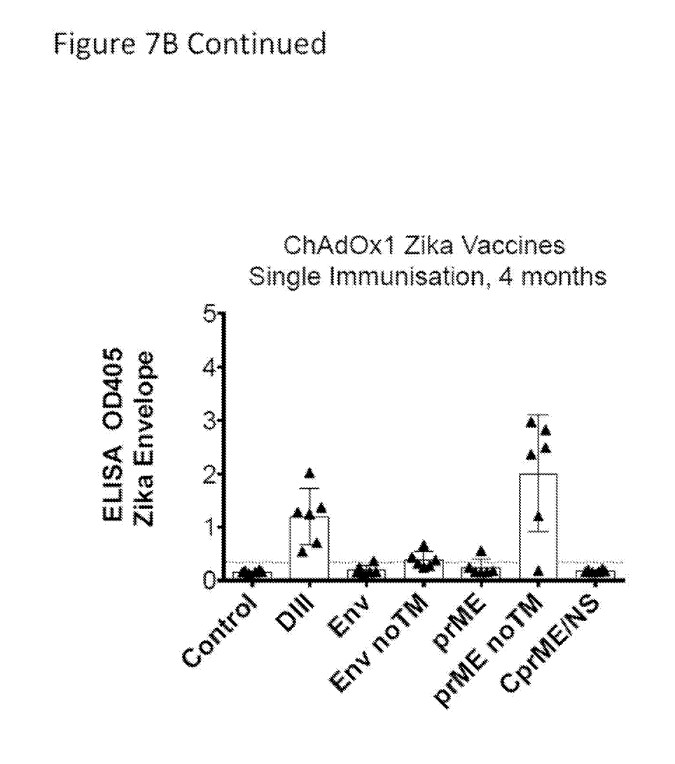

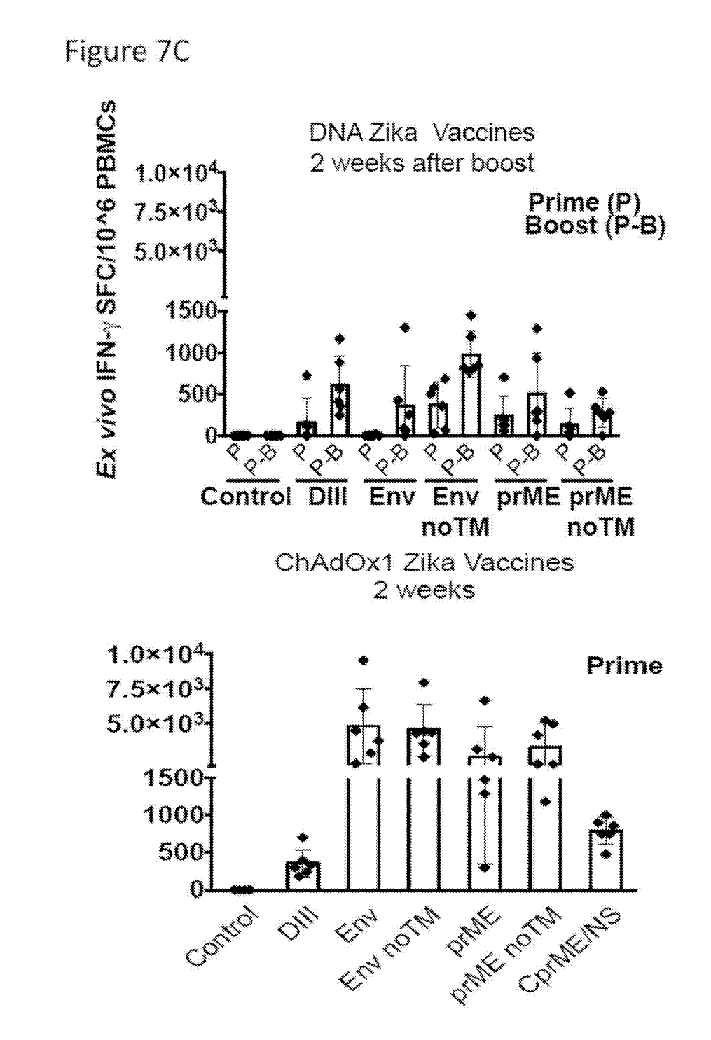

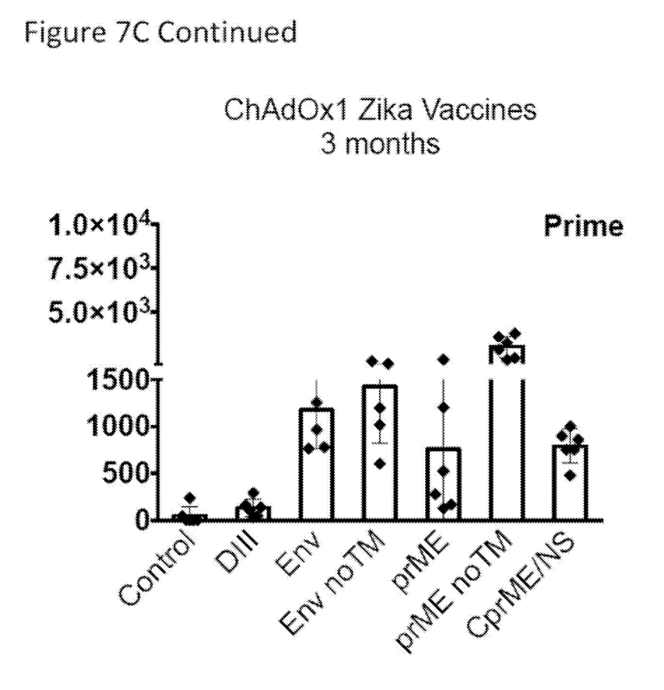

FIG. 7. Immune Responses Elicited by DNA vs ChAdOx1 vaccines. (A) For ZIKV DNA vaccines, BALB/c mice (n=6 per group) were immunised intramuscularly (i.m.) with a dose of 100 μg/mice, followed by a DNA Boost two weeks thereafter. For ChAdOx1 Zika vaccines, a single dose of 10⁸ IU/mice was i.m. administered. Blood samples were obtained at depicted time points for ELISA and ELISPOT assays. (B) Humoral responses elicited by DNA Prime-Boost after two weeks (left graph) and by a single immunisation of ChAdOx1 Zika vaccines at two weeks (right) and four months (bottom). Antibody responses were quantified by ELISA plates coated with ZIKV envelope protein. Error bar and bars represent the mean with SD. (C) PBMCs-INFγ producing cells from DNA Prime, DNA Prime-Boost after two weeks (left graph) and ChAdOx1 Zika vaccines at two weeks (right) and three months (bottom) after single immunisation were quantified by ELISPOT. 20mer peptides spanning the ZIKV envelope protein (10 μg/ml) were used for stimulation.

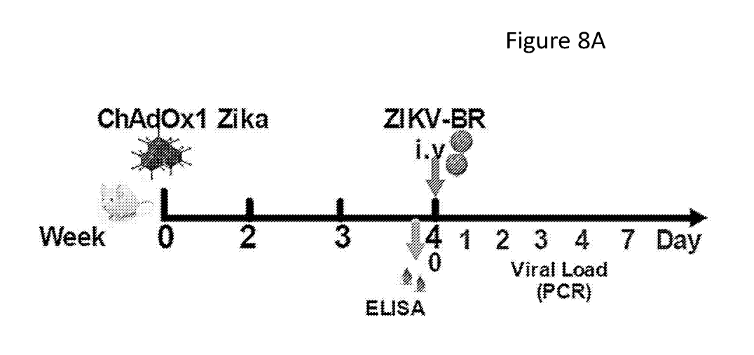

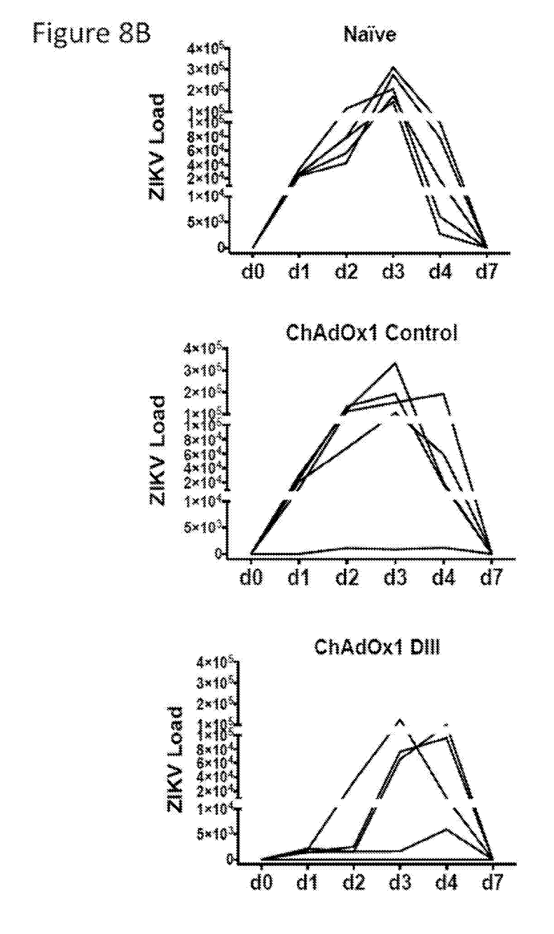

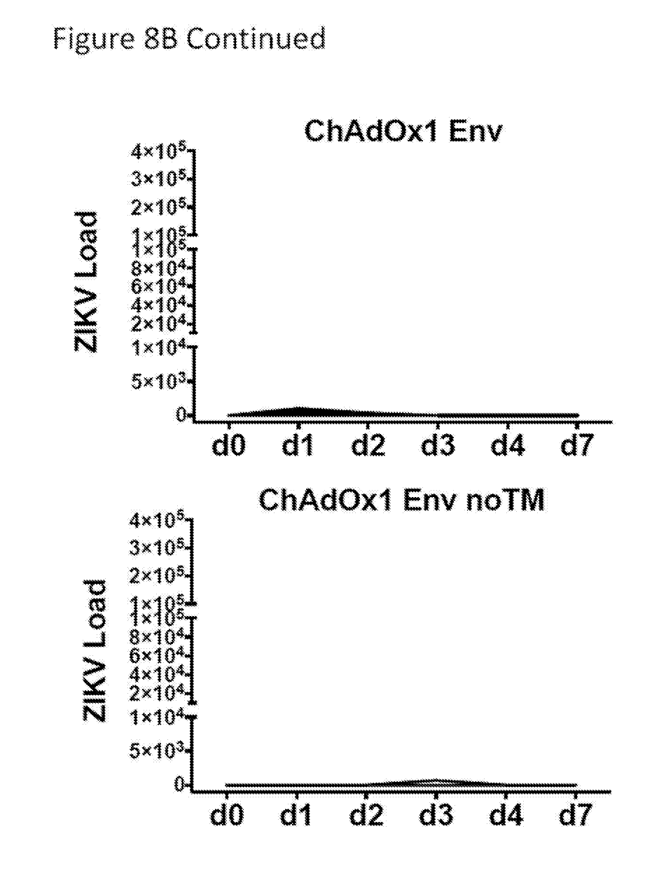

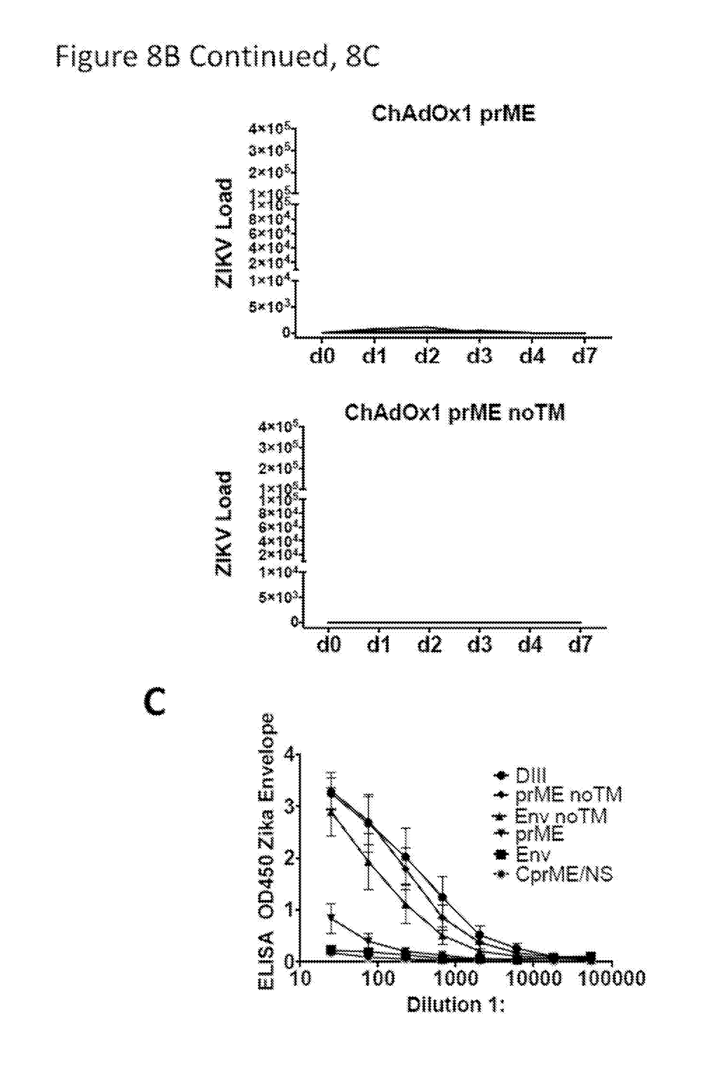

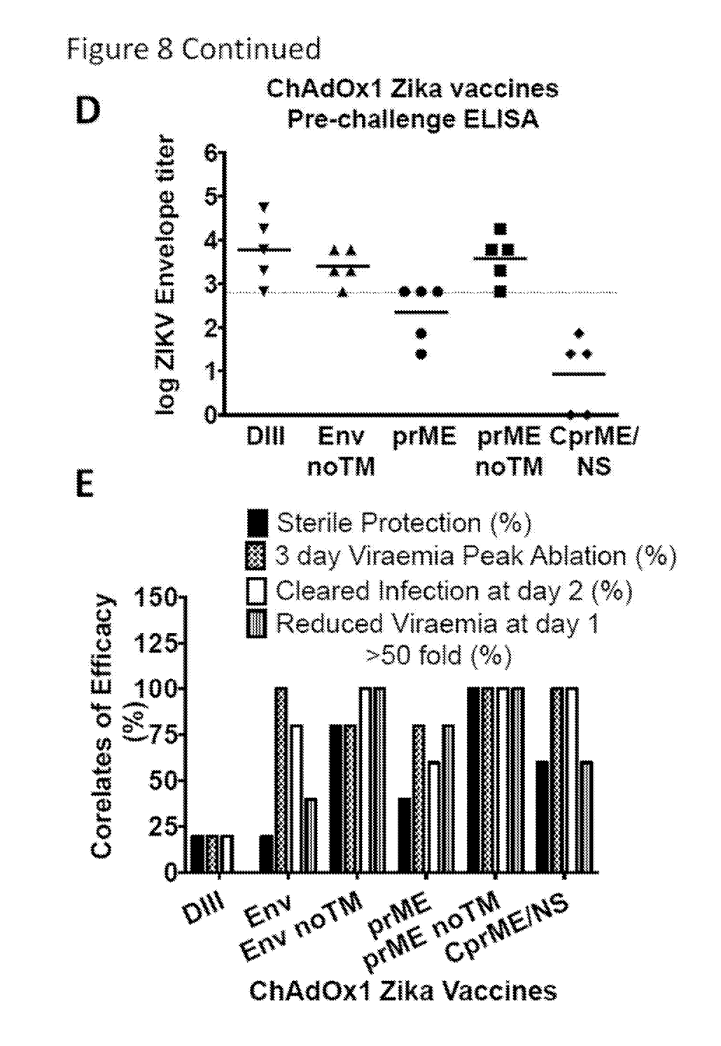

FIG. 8. Assessment of Protective Efficacy induced by ChAdOx1 Zika vaccines. (A) Balb/C mice (n=5) were immunised with a single i.m. shot of ChAdOx1 Zika vaccines and a ChAdOx1 unrelated vector were intravenously challenged with 105 VP of ZIKA-BR at week four after prime. (B) Viral load in vaccinated groups was monitored followed 7 days in sera to follow the onset of viraemia. (C) ELISA endpoint OD titers and (D) reciprocal ELISA titers of 4 weeks pre-challenge sera from vaccinated mice were calculated. (E) Vaccine efficacy scenarios observed in groups vaccinated with ChAdOx1 Zika vaccines

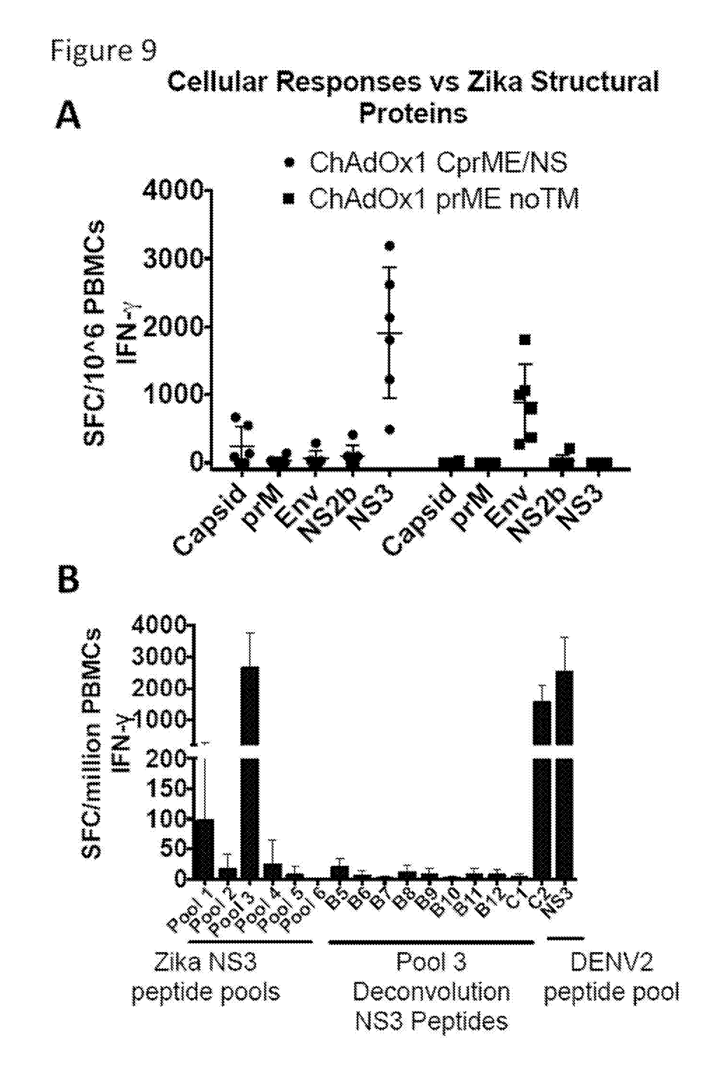

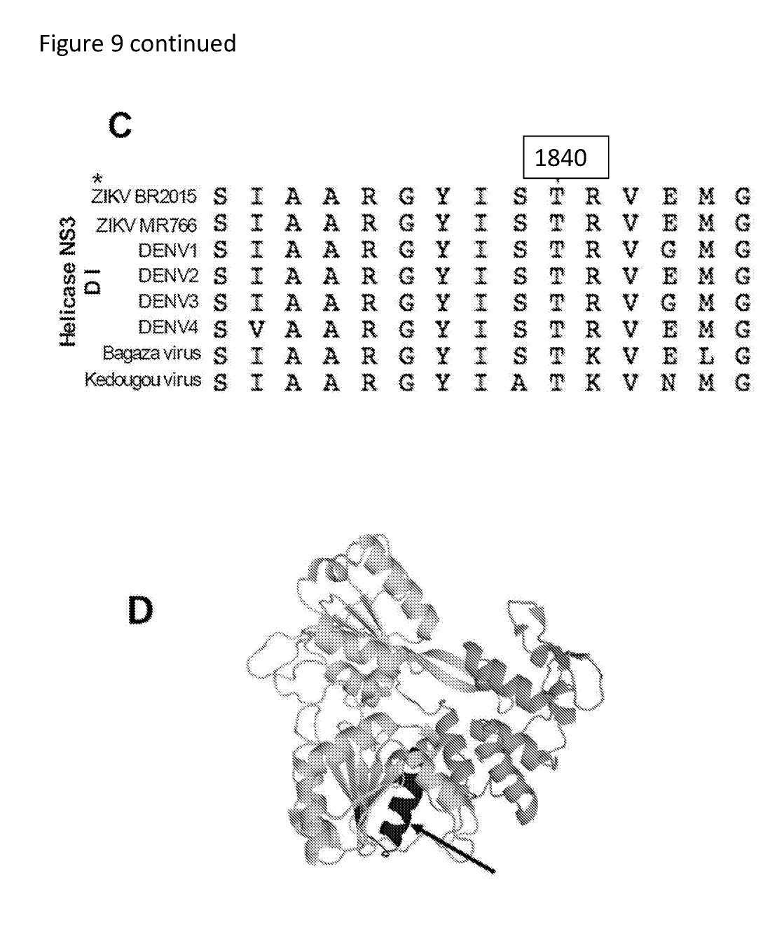

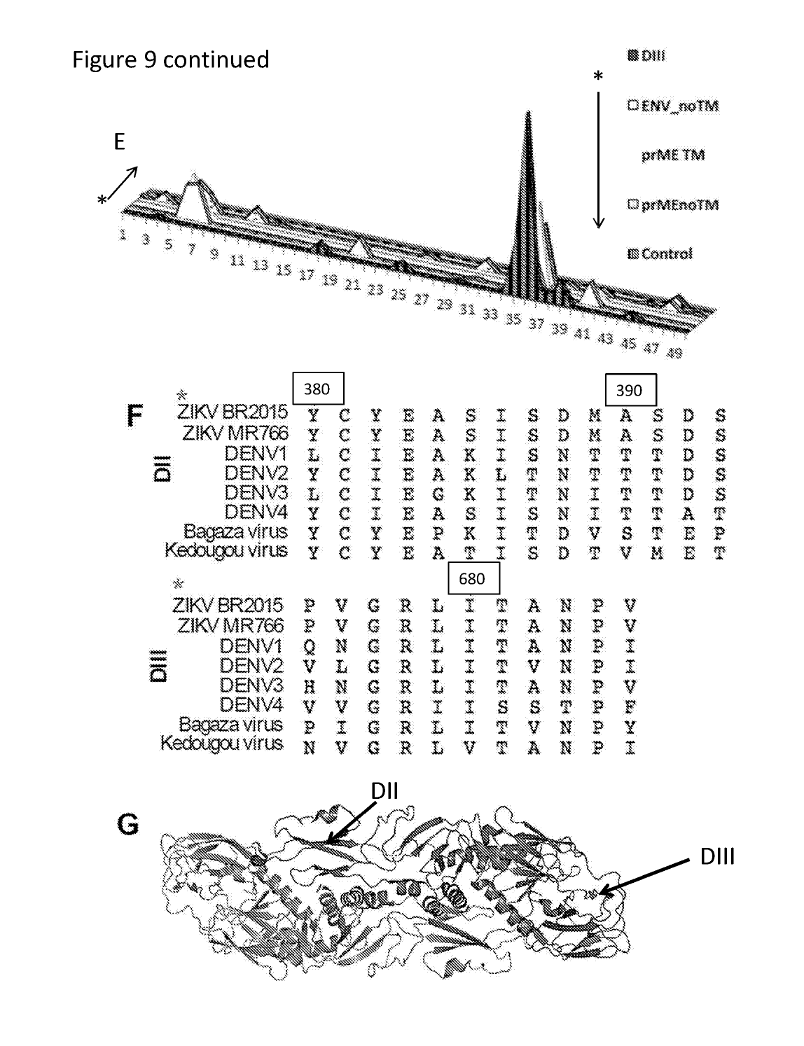

FIG. 9. T-Cell epitope mapping for NS3 and Envelope Zika (A) Peptide stimulation of peptides spanning all the proteins involved in the development of Zika vaccines. PBMCs from mice immunised with ChAdOx1 prME and CprME/NS were used for comparison. (B) DENV2 NS3 pools and Zika NS3 pools were assayed in ELISPOT to determine the immunodominant peptide and its homology with other flaviviruses (C), which was mapped in the Helicase domain I (alpha-helix (see arrow)) of Zika NS3 (D). (E) Zika envelope pools were also assayed to determine the immunodominant peptides along with their homology with other flaviviruses (F), which were mapped in the domain II (DII ribbon) and domain III (DIII loop) of Zika envelope (G).

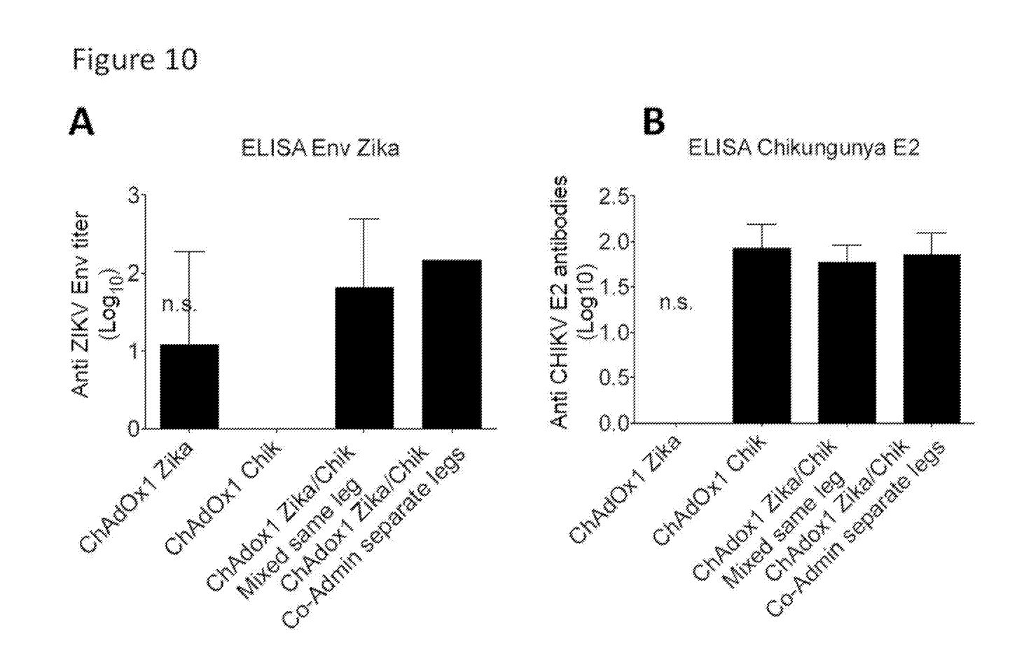

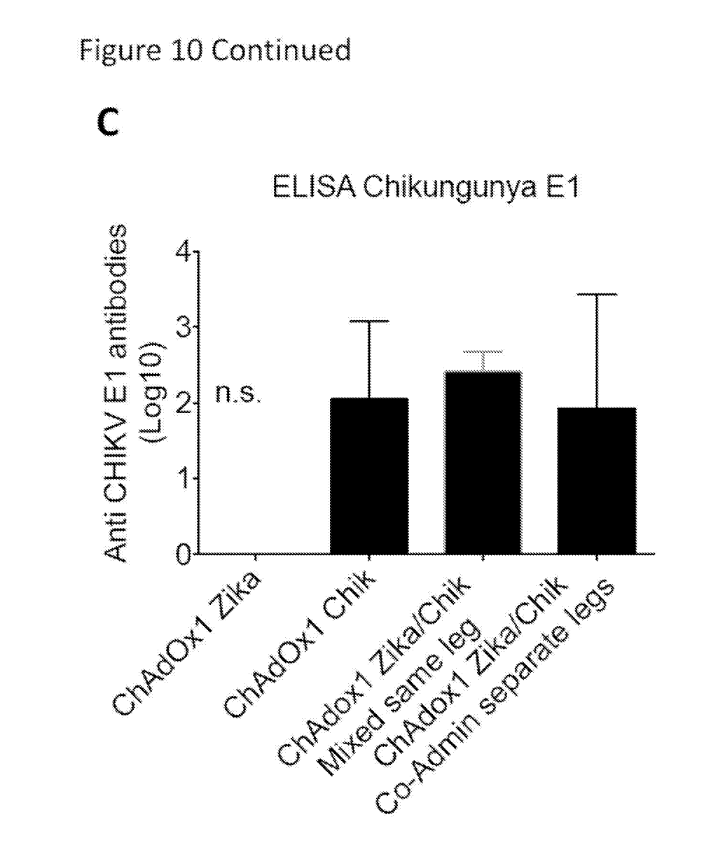

FIG. 10. Comparative immunogenicity against Zika and Chikungunya structural antigens, elicited by a bivalent vaccine. Antibody titers were compared between a single-component vaccine and a bivalent vaccine delivered as a mixture of two ChAdOx1-Zika/ChAdOx1-Chikungunya delivered in the same leg or a co-administration of both ChAdOx1-Zika+Chik applied in different legs. No statistical differences were observed.

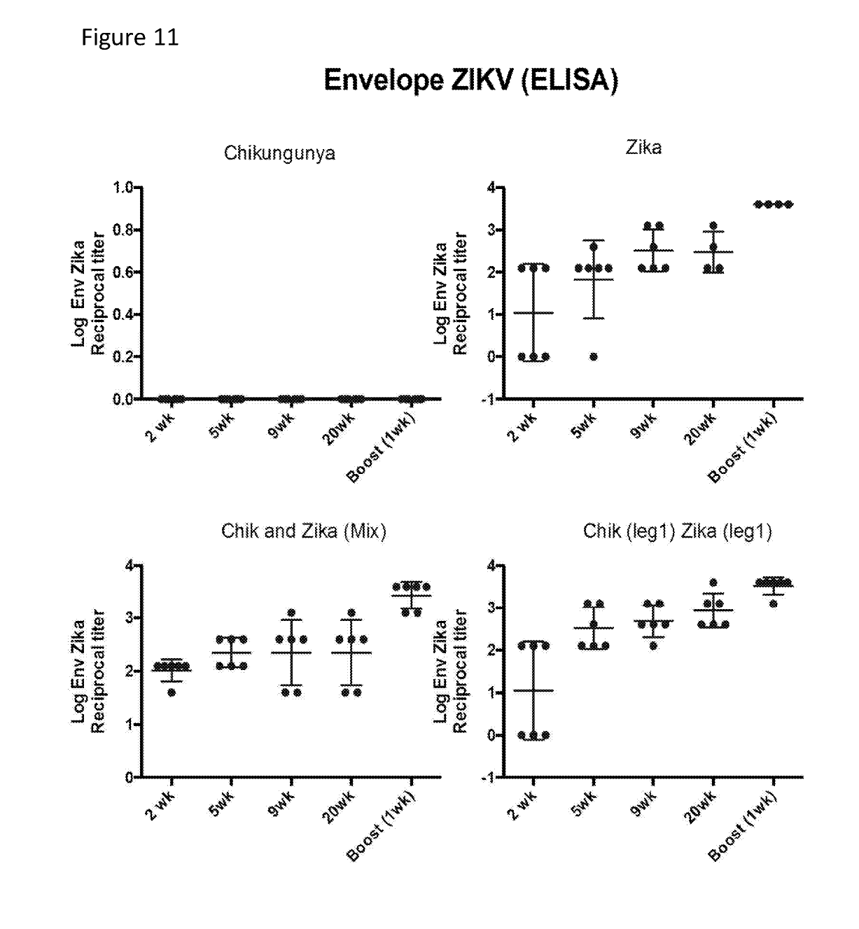

FIG. 11. Antibody responses against Zika virus envelope upon vaccination with a ChAdOx1-Zika vaccine alone or in combination of a ChAdOx1-Chikungunya vaccine as a mixture or co-vaccination in different legs.

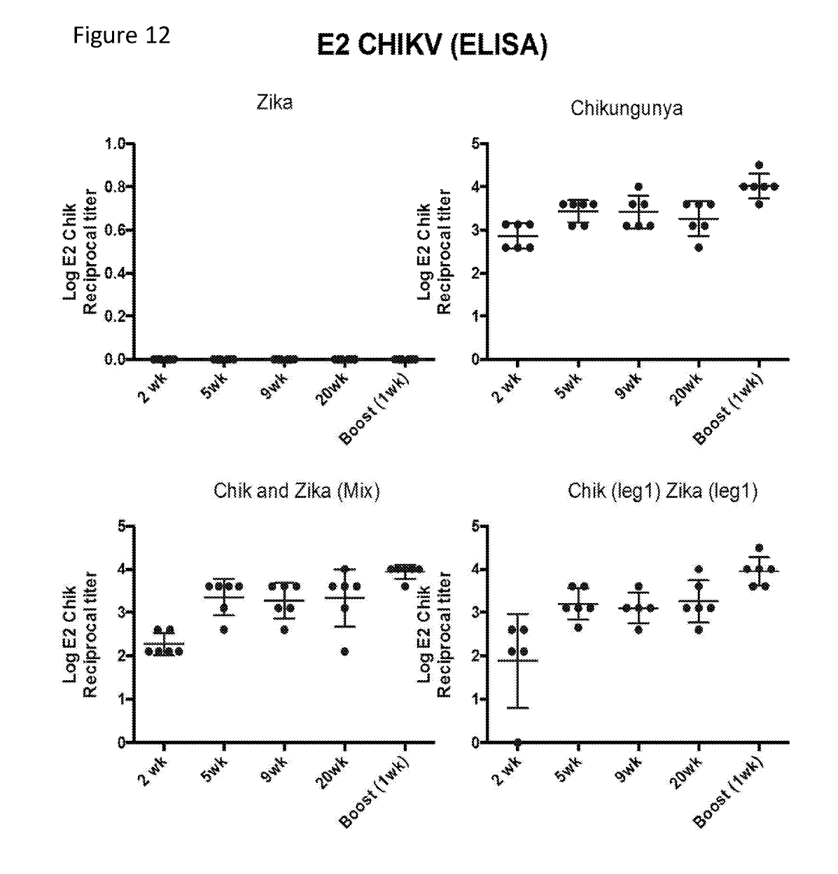

FIG. 12. Antibody responses against Chikungunya virus envelope 2 protein upon vaccination with a ChAdOx1-Chikungunya vaccine alone or in combination of a ChAdOx1-Zika vaccine as a mixture or co-vaccination in different legs.

### EXAMPLE 1

### Zika Virus Vaccine Development

Vaccine development is a lengthy process that requires careful selection of the best candidates to provide the best protection. Every pathogen's genetic sequence inserted into a new viral vectored vaccine will produce proteins that will follow various pathways of secretion depending on the leading sequences and presence of transmembrane regions. Thus the recombinant viruses described herein contain various versions of the ZIKV structural antigens with or without anchoring regions. This has a profound effect on immunogenicity and ultimately in protective efficacy. Therefore, it is important to study and carefully select all these variables in order to find the most efficacious vaccine, supported by the use of functional assays.

The Zika virus structural antigens have been carefully designed and consist on a consensus sequence derived from all Asian ZIKV genetic sequences reported in the literature. We obtained an immunogen with 98% homology to the ZIKV causing the current epidemics in the Americas. An antigen based on a consensus sequence will maximise coverage, yielding a vaccine that will be useful not only in endemic countries like Brazil but also in other affected regions in Asia and the potential to cover African Zika lineages. To minimise future issues of low immunogenicity in humans, we have constructed 5 variants of a ZIKV antigen to be used in 10 vaccines and we aim to apply functional assays to find the most immunogenic and protective vaccine, suitable for the clinic.

Zika vaccine candidates were constructed using a cassette expressing the Zika structural antigens, which contain the following regions: Pre-membrane (PrM) and Envelope (Env). All cassettes contained a 5′ leading sequence known as tPA, used in the Jenner ChAdOx vaccines to support secretion of the proteins once they are produced within cells. Two cassettes expressed the PrM structural antigen and three cassettes did not express the PrM.

Regarding the Env, two cassettes expressed the whole Env protein, which includes the domain I, II and III of Env and a transmembrane region(TM) located at the C-terminus region of the Env protein. Three cassettes did not contain such TM region, in order to further promote secretion of the protein to the extracellular milieu and stimulate antibody responses. The reasoning behind this is that the TM region could anchor a protein to cell membranes, preventing secretion. Finally, one cassette contained only the DIII region, which is part of the Env and the aim of this construct was to stimulate antibody responses only against the DIII, which is the domain used by the Zika virus to attach to cells. Anti-DIII antibodies may block and neutralise the virus and prevent attachment and entry, while at the same time, no induction of antibodies would take place against the rest of the protein, which has been involved in the antibody dependent enhancement (ADE), whereby antibodies against PrM, DI and DII enhance entry of virus rather than neutralisation, provoking higher viraemias and severity of the Zika or Dengue diseases (Zika could promote dengue ADE and vice versa).

### Sequences

Sequences, or encoded sequences, of the potential Zika viral vector components are described below. The Zika viral vector of the invention may comprise any one of the nucleic acid sequences provided below, or variants thereof. Alternatively, or additionally, the Zika viral vector of the invention may comprise nucleic acid encoding any one of the amino acid sequences provided below, or variants thereof.

The sequence of the antigenic component of the Zika viral vector of the invention (i.e. not including the viral vector backbone such as ChAdOx sequence) may consist essentially of one of the following sequences, or variants thereof (or sequences encoding the amino acid sequences, or variants thereof, where appropriate).

ZIKA Envelope Domain III (ZDIII) (Also known as ″DIII″)
(SEQ ID NO: 1)
ATGAAGATGGACAAGCTGCGGCTGAAGGGCGTGTCCTACAGCCTGTGTACCGCCGCCTT
CACCTTCACCAAGATCCCCGCCGAGACACTGCACGGCACCGTGACTGTGGAAGTGCAGT
ACGCCGGCACCGACGGCCCTTGTAAAGTGCCTGCTCAGATGGCCGTGGATATGCAGACC
CTGACCCCCGTGGGCAGACTGATCACCGCCAACCCTGTGATCACCGAGAGCACCGAGAA
CAGCAAGATGATGCTGGAACTGGACCCCCCCTTCGGCGACTCCTACATCGTGATCGGCG
TGGGAGAGAAGAAGATCACCCACCACTGGCACAGAAGCGGCAGCACCATCGGCAAG
Protein
(SEQ ID NO: 2)
MKMDKLRLKGVSYSLCTAAFTFTKIPAETLHGTVTVEVQYAGTDGPCKVPAQMA
VDMQTLTPVGRLITANPVITESTENSKMMLELDPPFGDSYIVIGVGEKKITHHW
HRSGSTIGK
ZIKA Envelope with no Transmembrane domain (ZENV_noTM)
(Also known as ″Env noTM″)
(SEQ ID NO: 3)
ATGCGGTGTATCGGCGTGTCCAACCGGGACTTCGTGGAAGGCATGAGCGGCGGCACATG
GGTGGACGTGGTGCTGGAACATGGCGGCTGCGTGACAGTGATGGCCCAGGACAAGCCCA
CCGTGGACATCGAGCTCGTGACCACCACCGTGTCCAATATGGCCGAAGTGCGGAGCTAC
TGCTACGAGGCCAGCATCAGCGACATGGCCAGCGACAGCAGATGCCCTACACAGGGCGA
GGCCTACCTGGATAAGCAGTCCGACACCCAGTACGTGTGCAAGCGGACCCTGGTGGATA
GAGGCTGGGGCAATGGCTGCGGCCTGTTTGGCAAGGGCAGCCTCGTGACCTGCGCCAAG
TTCGCCTGCAGCAAGAAGATGACCGGCAAGAGCATCCAGCCCGAGAACCTGGAATACCG
GATCATGCTGAGCGTGCACGGCAGCCAGCACTCCGGCATGATCGTGAACGACACCGGCC
ACGAGACAGACGAGAACCGGGCCAAGGTGGAAATCACCCCCAACAGCCCTAGAGCCGAG
GCCACCCTGGGCGGCTTTGGATCTCTGGGACTGGACTGCGAGCCCAGAACCGGCCTGGA
CTTCAGCGACCTGTACTACCTGACCATGAACAACAAGCACTGGCTGGTGCACAAAGAGT
GGTTCCACGACATCCCCCTGCCCTGGCATGCCGGCGCTGATACAGGCACACCCCACTGGA
ACAACAAAGAGGCTCTGGTGGAATTCAAGGACGCCCACGCCAAGCGGCAGACCGTGGTG
GTGCTGGGATCTCAGGAAGGCGCCGTGCATACAGCTCTGGCTGGCGCCCTGGAAGCCGA
AATGGATGGCGCCAAAGGCAGACTGTCCAGCGGCCACCTGAAGTGCCGGCTGAAGATGG
ACAAGCTGCGGCTGAAGGGCGTGTCCTACAGCCTGTGTACCGCCGCCTTCACCTTCACC
AAGATCCCCGCCGAGACACTGCACGGCACCGTGACTGTGGAAGTGCAGTACGCCGGCAC
CGACGGCCCTTGTAAAGTGCCTGCTCAGATGGCCGTGGATATGCAGACCCTGACCCCCG
TGGGCAGACTGATCACCGCCAACCCTGTGATCACCGAGAGCACCGAGAACAGCAAGATG
ATGCTGGAACTGGACCCCCCCTTCGGCGACTCCTACATCGTGATCGGCGTGGGAGAGAA
GAAGATCACCCACCACTGGCACAGAAGCGGCAGCACCATCGGCAAGGCCTTTGAGGCTA
CAGTGCGGGGAGCCAAGAGAATGGCCGTGCTGGGAGATACCGCCTGGGACTTTGGCTCT
GTGGGCGGAGCCCTGAACTCTCTG
Protein
(SEQ ID NO: 4)
MRCIGVSNRDFVEGMSGGTWVDVVLEHGGCVTVMAQDKPTVDIELVTTTVSNMA
EVRSYCYEASISDMASDSRCPTQGEAYLDKQSDTQYVCKRTLVDRGWGNGCGLF
GKGSLVTCAKFACSKKMTGKSIQPENLEYRIMLSVHGSQHSGMIVNDTGHETDE
NRAKVEITPNSPRAEATLGGFGSLGLDCEPRTGLDFSDLYYLTMNNKHWLVHKE
WFHDIPLPWHAGADTGTPHWNNKEALVEFKDAHAKRQTVVVLGSQEGAVHTALA
GALEAEMDGAKGRLSSGHLKCRLKMDKLRLKGVSYSLCTAAFTFTKIPAETLHG
TVTVEVQYAGTDGPCKVPAQMAVDMQTLTPVGRLITANPVITESTENSKMMLEL
DPPFGDSYIVIGVGEKKITHHWHRSGSTIGKAFEATVRGAKRMAVLGDTAWDFG
SVGGALNSL
ZIKA Envelope with Transmembrane domain (ZENV_TM)
(Also known as ″Env″)
(SEQ ID NO: 5)
ATGCGGTGTATCGGCGTGTCCAACCGGGACTTCGTGGAAGGCATGAGCGGCGGCACATG
GGTGGACGTGGTGCTGGAACATGGCGGCTGCGTGACAGTGATGGCCCAGGACAAGCCCA
CCGTGGACATCGAGCTCGTGACCACCACCGTGTCCAATATGGCCGAAGTGCGGAGCTAC
TGCTACGAGGCCAGCATCAGCGACATGGCCAGCGACAGCAGATGCCCTACACAGGGCGA
GGCCTACCTGGATAAGCAGTCCGACACCCAGTACGTGTGCAAGCGGACCCTGGTGGATA
GAGGCTGGGGCAATGGCTGCGGCCTGTTTGGCAAGGGCAGCCTCGTGACCTGCGCCAAG
TTCGCCTGCAGCAAGAAGATGACCGGCAAGAGCATCCAGCCCGAGAACCTGGAATACCG
GATCATGCTGAGCGTGCACGGCAGCCAGCACTCCGGCATGATCGTGAACGACACCGGCC
ACGAGACAGACGAGAACCGGGCCAAGGTGGAAATCACCCCCAACAGCCCTAGAGCCGAG
GCCACCCTGGGCGGCTTTGGATCTCTGGGACTGGACTGCGAGCCCAGAACCGGCCTGGA
CTTCAGCGACCTGTACTACCTGACCATGAACAACAAGCACTGGCTGGTGCACAAAGAGT
GGTTCCACGACATCCCCCTGCCCTGGCATGCCGGCGCTGATACAGGCACACCCCACTGGA
ACAACAAAGAGGCTCTGGTGGAATTCAAGGACGCCCACGCCAAGCGGCAGACCGTGGTG
GTGCTGGGATCTCAGGAAGGCGCCGTGCATACAGCTCTGGCTGGCGCCCTGGAAGCCGA
AATGGATGGCGCCAAAGGCAGACTGTCCAGCGGCCACCTGAAGTGCCGGCTGAAGATGG
ACAAGCTGCGGCTGAAGGGCGTGTCCTACAGCCTGTGTACCGCCGCCTTCACCTTCACC
AAGATCCCCGCCGAGACACTGCACGGCACCGTGACTGTGGAAGTGCAGTACGCCGGCAC
CGACGGCCCTTGTAAAGTGCCTGCTCAGATGGCCGTGGATATGCAGACCCTGACCCCCG
TGGGCAGACTGATCACCGCCAACCCTGTGATCACCGAGAGCACCGAGAACAGCAAGATG
ATGCTGGAACTGGACCCCCCCTTCGGCGACTCCTACATCGTGATCGGCGTGGGAGAGAA
GAAGATCACCCACCACTGGCACAGAAGCGGCAGCACCATCGGCAAGGCCTTTGAGGCTA
CAGTGCGGGGAGCCAAGAGAATGGCCGTGCTGGGAGATACCGCCTGGGACTTTGGCTCT
GTGGGCGGAGCCCTGAACTCTCTGGGCAAGGGAATCCACCAGATCTTCGGCGCTGCCTT
CAAGAGCCTGTTCGGCGGCATGAGCTGGTTCAGCCAGATCCTGATCGGCACCCTGCTGA
TGTGGCTGGGCCTGAACACCAAGAACGGCAGCATCTCCCTGATGTGCCTGGCTCTGGGA
GGCGTGCTGATCTTCCTGAGCACAGCCGTGTCCGCC
Protein
(SEQ ID NO: 6)
MRCIGVSNRDFVEGM8GGTWVDVVLEHGGCVTVMAQDKPTVDIELVTTTVSNMA
EVRSYCYEASISDMASDSRCPTQGEAYLDKQSDTQYVCKRTLVDRGWGNGCGLF
GKGSLVTCAKFACSKKMTGKSIQPENLEYRIMLSVHGSQHSGMIVNDTGHETDE
NRAKVEITPNSPRAEATLGGFGSLGLDCEPRTGLDFSDLYYLTMNNKHWLVHKE
WFHDIPLPWHAGADTGTPHWNNKEALVEFKDAHAKRQTVVVLGSQEGAVHTALA
GALEAEMDGAKGRLSSGHLKCRLKMDKLRLKGVSYSLCTAAFTFTKIPAETLHG
TVTVEVQYAGTDGPCKVPAQMAVDMQTLTPVGRLITANPVITESTENSKMMLEL
DPPFGDSYIVIGVGEKKITHHWHRSGSTIGKAFEATVRGAKRMAVLGDTAWDFG
SVGGALNSLGKGIHQIFGAAFKSLFGGMSWFSQILIGTLLMWLGLNTKNGSISL
MCLALGGVLIFLSTAVSA
ZIKA prM Envelope with no Transmembrane domain (ZprMENV_noTM)
(Also known as ″prME no TM″)
(SEQ ID NO: 7)
ACAAGACGGGGCAGCGCCTACTACATGTACCTGGACAGAAACGACGCCGGCGAGGCCAT
CAGCTTCCCTACCACACTGGGCATGAACAAGTGCTACATCCAGATCATGGACCTGGGCC
ACATGTGCGACGCCACAATGAGCTACGAGTGCCCCATGCTGGACGAGGGCGTGGAACCC
GACGATGTGGACTGCTGGTGCAACACCACCAGCACCTGGGTGGTGTACGGCACCTGTCA
CCACAAGAAGGGCGAAGCCAGACGGTCCAGACGGGCCGTGACACTGCCTAGCCACAGCA
CCAGAAAGCTGCAGACCCGGTCCCAGACCTGGCTGGAAAGCAGAGAGTACACCAAGCAC
CTGATCCGGGTGGAAAACTGGATCTTCCGGAACCCCGGCTTTGCCCTGGCCGCTGCTGC
TATTGCTTGGCTGCTGGGCAGCTCCACCTCCCAGAAAGTGATCTACCTCGTGATGATCC
TGCTGATCGCCCCTGCCTACAGCATCCGGTGTATCGGCGTGTCCAACCGGGACTTCGTG
GAAGGCATGAGCGGCGGCACATGGGTGGACGTGGTGCTGGAACATGGCGGCTGCGTGAC
AGTGATGGCCCAGGACAAGCCCACCGTGGACATCGAGCTCGTGACCACCACCGTGTCCA
ATATGGCCGAAGTGCGGAGCTACTGCTACGAGGCCAGCATCAGCGACATGGCCAGCGAC
AGCAGATGCCCTACACAGGGCGAGGCCTACCTGGATAAGCAGTCCGACACCCAGTACGT
GTGCAAGCGGACCCTGGTGGATAGAGGCTGGGGCAATGGCTGCGGCCTGTTTGGCAAGG
GCAGCCTCGTGACCTGCGCCAAGTTCGCCTGCAGCAAGAAGATGACCGGCAAGAGCATC
CAGCCCGAGAACCTGGAATACCGGATCATGCTGAGCGTGCACGGCAGCCAGCACTCCGG
CATGATCGTGAACGACACCGGCCACGAGACAGACGAGAACCGGGCCAAGGTGGAAATCA
CCCCCAACAGCCCTAGAGCCGAGGCCACCCTGGGCGGCTTTGGATCTCTGGGACTGGAC
TGCGAGCCCAGAACCGGCCTGGACTTCAGCGACCTGTACTACCTGACCATGAACAACAA
GCACTGGCTGGTGCACAAAGAGTGGTTCCACGACATCCCCCTGCCCTGGCATGCCGGCG
CTGATACAGGCACACCCCACTGGAACAACAAAGAGGCTCTGGTGGAATTCAAGGACGCC
CACGCCAAGCGGCAGACCGTGGTGGTGCTGGGATCTCAGGAAGGCGCCGTGCATACAGC
TCTGGCTGGCGCCCTGGAAGCCGAAATGGATGGCGCCAAAGGCAGACTGTCCAGCGGCC
ACCTGAAGTGCCGGCTGAAGATGGACAAGCTGCGGCTGAAGGGCGTGTCCTACAGCCTG
TGTACCGCCGCCTTCACCTTCACCAAGATCCCCGCCGAGACACTGCACGGCACCGTGACT
GTGGAAGTGCAGTACGCCGGCACCGACGGCCCTTGTAAAGTGCCTGCTCAGATGGCCGT
GGATATGCAGACCCTGACCCCCGTGGGCAGACTGATCACCGCCAACCCTGTGATCACCG
AGAGCACCGAGAACAGCAAGATGATGCTGGAACTGGACCCCCCCTTCGGCGACTCCTAC
ATCGTGATCGGCGTGGGAGAGAAGAAGATCACCCACCACTGGCACAGAAGCGGCAGCAC
CATCGGCAAGGCCTTTGAGGCTACAGTGCGGGGAGCCAAGAGAATGGCCGTGCTGGGAG
ATACCGCCTGGGACTTTGGCTCTGTGGGCGGAGCCCTGAACTCTCTG
Protein
(SEQ ID NO: 8)
TRRGSAVYMYLDRNDAGEAISFPTTLGMNKCYIQIMDLGHMCDATMSYECPMLD
EGVEPDDVDCWCNTTSTWVVYGTCHHKKGEARRSRRAVTLPSHSTRKLQTRSQT
WLESREYTKHLIRVENWIFRNPGFALAAAAIAWLLGSSTSQKVIYLVMILLIAP
AYSIRCIGVSNRDFVEGMSGGTWVDVVLEHGGSVTVMAQDKPTVDIELVTTTVS
NMAEVRSYCYEASISDMASDSRCPTQGEAYLDKQSDTQYVCKRTLVDRGWGNGC
GLFGKGSLVTCAKFACSKKMTGKSIQPENLEYRIMLSVHGSQHSGMIVNDTGHE
TDENRAKVEITPNSPRAEATLGGFGSLGLDCEPRTGLDFSDLYYLTMNNKHWLV
HKEWFHDIPLPWHAGADTGTPHWNNKEALVEFKDAHAKRQTVVVLGSQEGAVHT
ALAGALEAEMDGAKGRLSSGHLKCRLKMDKLRLKGVSYSLCTAAFTFTKIPAET
LHGTVTVEVQYAGTDGPCKVPAQMAVDMQTLTPVGRLITANPVITESTENSKMM
LELDPPFGDSYIVIGVGEKKITHHWHRSGSTIGKAFEATVRGAKRMAVLGDTAW
DFGSVGGALNSL
(PrM sequence is underlined)
ZIKA prM Envelope with Transmembrane domain (ZprMENV_TM)
(Also known as ″prME″)
(SEQ ID NO: 9)
ACAAGACGGGGCAGCGCCTACTACATGTACCTGGACAGAAACGACGCCGGCGAGGCCAT
CAGCTTCCCTACCACACTGGGCATGAACAAGTGCTACATCCAGATCATGGACCTGGGCC
ACATGTGCGACGCCACAATGAGCTACGAGTGCCCCATGCTGGACGAGGGCGTGGAACCC
GACGATGTGGACTGCTGGTGCAACACCACCAGCACCTGGGTGGTGTACGGCACCTGTCA
CCACAAGAAGGGCGAAGCCAGACGGTCCAGACGGGCCGTGACACTGCCTAGCCACAGCA
CCAGAAAGCTGCAGACCCGGTCCCAGACCTGGCTGGAAAGCAGAGAGTACACCAAGCAC
CTGATCCGGGTGGAAAACTGGATCTTCCGGAACCCCGGCTTTGCCCTGGCCGCTGCTGC
TATTGCTTGGCTGCTGGGCAGCTCCACCTCCCAGAAAGTGATCTACCTCGTGATGATCC
TGCTGATCGCCCCTGCCTACAGCATCCGGTGTATCGGCGTGTCCAACCGGGACTTCGTG
GAAGGCATGAGCGGCGGCACATGGGTGGACGTGGTGCTGGAACATGGCGGCTGCGTGAC
AGTGATGGCCCAGGACAAGCCCACCGTGGACATCGAGCTCGTGACCACCACCGTGTCCA
ATATGGCCGAAGTGCGGAGCTACTGCTACGAGGCCAGCATCAGCGACATGGCCAGCGAC
AGCAGATGCCCTACACAGGGCGAGGCCTACCTGGATAAGCAGTCCGACACCCAGTACGT
GTGCAAGCGGACCCTGGTGGATAGAGGCTGGGGCAATGGCTGCGGCCTGTTTGGCAAGG
GCAGCCTCGTGACCTGCGCCAAGTTCGCCTGCAGCAAGAAGATGACCGGCAAGAGCATC
CAGCCCGAGAACCTGGAATACCGGATCATGCTGAGCGTGCACGGCAGCCAGCACTCCGG
CATGATCGTGAACGACACCGGCCACGAGACAGACGAGAACCGGGCCAAGGTGGAAATCA
CCCCCAACAGCCCTAGAGCCGAGGCCACCCTGGGCGGCTTTGGATCTCTGGGACTGGAC
TGCGAGCCCAGAACCGGCCTGGACTTCAGCGACCTGTACTACCTGACCATGAACAACAA
GCACTGGCTGGTGCACAAAGAGTGGTTCCACGACATCCCCCTGCCCTGGCATGCCGGCG
CTGATACAGGCACACCCCACTGGAACAACAAAGAGGCTCTGGTGGAATTCAAGGACGCC
CACGCCAAGCGGCAGACCGTGGTGGTGCTGGGATCTCAGGAAGGCGCCGTGCATACAGC
TCTGGCTGGCGCCCTGGAAGCCGAAATGGATGGCGCCAAAGGCAGACTGTCCAGCGGCC
ACCTGAAGTGCCGGCTGAAGATGGACAAGCTGCGGCTGAAGGGCGTGTCCTACAGCCTG
TGTACCGCCGCCTTCACCTTCACCAAGATCCCCGCCGAGACACTGCACGGCACCGTGACT
GTGGAAGTGCAGTACGCCGGCACCGACGGCCCTTGTAAAGTGCCTGCTCAGATGGCCGT
GGATATGCAGACCCTGACCCCCGTGGGCAGACTGATCACCGCCAACCCTGTGATCACCG
AGAGCACCGAGAACAGCAAGATGATGCTGGAACTGGACCCCCCCTTCGGCGACTCCTAC
ATCGTGATCGGCGTGGGAGAGAAGAAGATCACCCACCACTGGCACAGAAGCGGCAGCAC
CATCGGCAAGGCCTTTGAGGCTACAGTGCGGGGAGCCAAGAGAATGGCCGTGCTGGGAG
ATACCGCCTGGGACTTTGGCTCTGTGGGCGGAGCCCTGAACTCTCTGGGCAAGGGAATC
CACCAGATCTTCGGCGCTGCCTTCAAGAGCCTGTTCGGCGGCATGAGCTGGTTCAGCCA
GATCCTGATCGGCACCCTGCTGATGTGGCTGGGCCTGAACACCAAGAACGGCAGCATCT
CCCTGATGTGCCTGGCTCTGGGAGGCGTGCTGATCTTCCTGAGCACAGCCGTGTCCGCC
Protein
(SEQ ID NO: 10)
TRRGSAYYMYLDRNDAGEAISFPTTLGMNKCYIQIMDLGHMCDATMSYECPMLD
EGVEPDDVDCWCNTTSTWVVYGTCHHKKGEARRSRRAVTLPSHSTRKLQTRSQT
WLESREYTKHLIRVENWIFRNPGFALAAAAIAWLLGSSTSQKVIYLVMILLIAP
AYSIRCIGVSNRDFVEGMSGGTWVDVVLEHGGCVTVMAQDKPTVDIELVTTTVS
NMAEVRSYCYEASISDMASDSRCPTQGEAYLDKQSDTQYVCKRTLVDRGWGNGC
GLFGKGSLVTCAKFACSKKMTGKSIQPENLEYRIMLSVHGSQHSGMIVNDTGHE
TDENRAKVEITPNSPRAEATLGGFGSLGLDCEPRTGLDFSDLYYLTMNNKHWLV
HKEWFHDIPLPWHAGADTGTPHWNNKEALVEFKDAHAKRQTVVVLGSQEGAVHT
ALAGALEAEMDGAKGRLSSGHLKCRLKMDKLRLKGVSYSLCTAAFTFTKIPAET
LHGTVTVEVQYAGTDGPCKVPAQMAVDMQTLTPVGRLITANPVITESTENSKMM
LELDPPFGDSYIVIGVGEKKITHHWHRSGSTIGKAFEATVRGAKRMAVLGDTAW
DFGSVGGALNSLGKGIHQIFGAAFKSLFGGMSWFSQILIGTLLMWLGLNTKNGS
ISLMCLALGGVLIFLSTAVSA
TPA 5′ leader sequence:
(SEQ ID NO: 11)
MDAMKRGLCCVLLLCGAVFVSPSQEIHARFRR
Shark invariant chain sequence
(SEQ ID NO: 12)
SLLWGGVTVLAAMLIAGQVASSVVFLV
pRM amino acid sequence:
(SEQ ID NO: 13)
TRRGSAYYMYLDRNDAGEAISFPTTLGMNKCYIQIMDLGHMCDATMSYECPMLD
EGVEPDDVDCWCNTTSTWVVYGTCHHKKGEARRSRRAVTLPSHSTRKLQTRSQT
WLESREYTKHLIRVENWIFRNPGFALAAAAIAWLLGSSTSQKVIYLVMILLIAP
AYS

### CprME/NS (Comprises Capsid, prM and Envelope Directly Followed by NS2b (a Cofactor of NS3), and NS3 (with Enzymatic Activity to cleave Capsid from prME)

This construct is more similar to the African Lineage, but still provides good protection in a challenge model with Asian Zika isolate (Brazilian)

DNA sequence
(SEQ ID NO: 18)
ACCAT
GAAGAACCCCAAGAAGAAGTCCGGCGGCTTCCGGATCGTGAACATGCTGA
AACGGGGCGTGGCCAGAGTGAACCCTCTGGGCGGACTGAAGAGACTGCCT
GCCGGACTGCTGCTGGGCCACGGCCCTATTAGAATGGTGCTGGCCATCCT
GGCCTTTCTGCGGTTCACCGCCATCAAGCCTAGCCTGGGCCTGATCAACA
GATGGGGCAGCGTGGGCAAGAAAGAAGCCATGGAAATCATCAAGAAGTTC
AAGAAAGACCTGGCCGCCATGCTGCGGATCATCAACGCCCGGAAAGAGCG
GAAGCGGAGAGGCGCCGATACCAGCATCGGCATCATTGGCCTGCTGCTGA
CCACAGCCATGGCCGCCGAGATCACCAGAAGAGGCAGCGCCTACTACATG
TACCTGGACAGAAGCGACGCCGGCAAGGCCATCAGCTTTGCCACAACCCT
GGGCGTGAACAAGTGCCACGTGCAGATCATGGACCTGGGCCACATGTGCG
ACGCCACAATGAGCTACGAGTGCCCCATGCTGGACGAGGGCGTGGAACCC
GACGATGTGGACTGCTGGTGCAACACCACCAGCACCTGGGTGGTGTACGG
CACCTGTCACCACAAGAAGGGCGAGGCCAGACGGTCTAGAAGGGCCGTGA
CACTGCCTAGCCACAGCACCCGGAAGCTGCAGACCAGAAGCCAGACCTGG
CTGGAAAGCAGAGAGTACACCAAGCACCTGATCAAGGTGGAAAACTGGAT
CTTCCGGAACCCCGGCTTCGCCCTGGCTGCCGTGGCTATTGCTTGGCTGC
TGGGAAGCAGCACCAGCCAGAAAGTGATCTACCTCGTGATGATCCTGCTG
ATCGCCCCTGCCTACAGCATCCGGTGTATCGGCGTGTCCAACCGGGACTT
CGTGGAAGGCATGAGCGGCGGCACATGGGTGGACGTGGTGCTGGAACATG
GCGGCTGCGTGACAGTGATGGCCCAGGACAAGCCCACCGTGGACATCGAG
CTCGTGACCACCACCGTGTCCAATATGGCCGAAGTGCGGAGCTACTGCTA
CGAGGCCAGCATCAGCGACATGGCCAGCGACAGCAGATGCCCTACACAGG
GGGAGGCCTACCTGGATAAGCAGTCCGACACCCAGTACGTGTGCAAGCGG
ACCCTGGTGGATAGAGGCTGGGGCAATGGCTGCGGCCTGTTTGGCAAGGG
CAGCCTCGTGACCTGCGCCAAGTTCACCTGTAGCAAGAAGATGACCGGCA
AGAGCATCCAGCCCGAGAACCTGGAATACCGGATCATGCTGAGCGTGCAC
GGCTCCCAGCACAGCGGCATGATCGTGAATGACATCGGCCACGAGACAGA
CGAGAACCGGGCCAAAGTGGAAGTGACCCCCAACAGCCCTAGAGCCGAGG
CCACACTGGGCGGCTTTGGATCTCTGGGCCTGGACTGCGAGCCTAGAACC
GGCCTGGATTTCAGCGACCTGTACTACCTGACCATGAACAACAAACACTG
GCTGGTGCACAAAGAGTGGTTCCACGACATCCCCCTGCCCTGGCATGCTG
GCGCTGATACAGGCACCCCCCACTGGAACAACAAAGAGGCCCTGGTGGAG
TTCAAGGACGCCCACGCCAAGAGGCAGACCGTGGTGGTGCTGGGATCTCA
GGAAGGCGCCGTGCATACAGCTCTGGCTGGCGCCCTGGAAGCCGAAATGG
ATGGCGCTAAGGGCCGGCTGTTTAGCGGCCACCTGAAGTGCCGGCTGAAG
ATGGACAAGCTGCGGCTGAAGGGCGTGTCCTACAGCCTGTGTACCGCCGC
CTTCACCTTCACCAAGGTGCCCGCCGAAACCCTGCACGGCACAGTGACTG
TGGAAGTGCAGTACGCCGGCACCGACGGCCCTTGTAAAGTGCCTGCTCAG
ATGGCCGTGGATATGCAGACCCTGACCCCCGTGGGCAGACTGATCACCGC
CAACCCTGTGATCACCGAGAGCACCGAGAACAGCAAGATGATGCTGGAAC
TGGACCCCCCCTTCGGCGACTCCTACATCGTGATCGGCGTGGGAGACAAG
AAGATCACCCACCACTGGCACCGCAGCGGCAGCACAATCGGAAAGGCCTT
CGAAGCCACAGTGCGGGGAGCCAAGAGAATGGCCGTGCTGGGCGATACCG
CCTGGGATTTTGGCTCTGTGGGCGGCGTGTTCAACTCCCTGGGCAAGGGA
ATCCACCAGATCTTCGGAGCCGCCTTTAAGAGCCTGTTCGGCGGCATGAG
CTGGTTCAGCCAGATCCTGATCGGCACCCTGCTCGTGTGGCTGGGACTGA
ACACCAAGAACGGCAGCATCTCCCTGACCTGCCTGGCTCTGGGGGGAGTG
ATGATCTTCCTGAGCACCGCCGTGTCCGCCCCTAGCGAAGTGCTGACAGC
CGTGGGACTGATCTGCGCTCTGGCAGGCGGATTCGCCAAGGCCGACATTG
AGATGGCCGGACCCATGGCTGCTGTGGGACTGCTGATTGTGTCCTACGTG
GTGTCCGGCAAGTCTGTGGACATGTACATCGAGAGAGCCGGCGACATCAC
CTGGGAGAAGGACGCCGAAGTGACAGGCAACAGCCCCAGACTGGACGTGG
CCCTGGATGAGAGCGGCGATTTCAGTCTGGTGGAAGAGGACGGCCCTCCC
ATGCGCGAGATCATTCTGAAAGTGGTGCTGATGGCAATCTGCGGGATGAA
CCCTATCGCCATCCCCTTCGCTGCCGGCGCTTGGTACGTGTACGTGAAAA
CAGGCAAGCGGAGCGGAGCCCTGTGGGATGTGCCTGCCCCCAAAGAAGTG
AAGAAAGGCGAGACAACCGACGGCGTGTACAGAGTGATGACCCGCAGACT
GCTGGGCAGCACACAAGTGGGAGTGGGCGTGATGCAGGAAGGGGTGTTCC
ACACCATGTGGCACGTGACCAAAGGCGCCGCTCTGAGATCTGGCGAGGGC
AGGCTGGATCCTTACTGGGGCGACGTGAAGCAGGACCTGGTGTCCTATTG
CGGCCCTTGGAAGCTGGACGCCGCTTGGGATGGACTGAGCGAGGTGCAGC
TGCTGGCTGTGCCTCCTGGCGAGAGGGCCAGAAACATCCAGACCCTGCCA
GGCATCTTCAAGACCAAGGACGGGGACATCGGCGCCGTGGCTCTGGATTA
TCCTGCCGGCACAAGCGGCTCCCCCATCCTGGACAAGTGTGGCAGAGTGA
TCGGCCTGTACGGCAACGGCGTCGTGATCAAGAATGGCAGCTATGTGTCC
GCCATCACCCAGGGCAAGCGGGAAGAGGAAACCCCTGTGGAATGCTTCGA
GCCCTCCATGCTGAAGAAAAAGCAGCTGACCGTGCTGGACCTGCACCCTG
GCGCCGGAAAAACCAGAAGGGTGCTGCCTGAGATCGTGCGGGAAGCCATC
AAGAAACGGCTGAGAACCGTGATCCTGGCCCCCACCAGAGTGGTGGCTGC
CGAGATGGAAGAAGCCCTGAGAGGACTGCCCGTGCGGTACATGACAACCG
CCGTGAACGTGACCCACTCTGGCACCGAGATCGTGGATCTGATGTGTCAC
GCCACCTTCACAAGCCGGCTGCTGCAGCCCATCCGGGTGCCCAACTACAA
CCTGTACATCATGGACGAGGCCCACTTCACCGACCCCAGCTCCATTGCCG
CCAGAGGCTACATCAGCACACGGGTGGAAATGGGCGAAGCTGCCGCCATC
TTCATGACCGCCACACCTCCCGGAACCAGGGACGCCTTCCCCGACAGCAA
CTCCCCTATCATGGACACCGAGGTGGAAGTGCCCGAGAGAGCCTGGTCCA
GCGGCTTCGACTGGGTCACAGATCACTCCGGCAAGACCGTGTGGTTCGTG
CCCTCTGTGCGGAACGGCAATGAGATCGCCGCCTGTCTGACAAAGGCCGG
GAAGAGAGTGATCCAGCTGAGCCGCAAGACCTTCGAGACAGAGTTCCAGA
AAACAAAGAACCAGGAATGGGATTTCGTGATCACCACAGACATCTCCGAG
ATGGGCGCCAACTTCAAGGCCGATCGCGTGATCGACAGCCGGCGGTGTCT
GAAGCCCGTGATTCTGGACGGCGAAAGAGTGATTCTGGCCGGACCTATGC
CCGTGACCCATGCCTCTGCCGCTCAGAGAAGAGGCCGGATCGGCAGAAAC
CCCAACAAGCCCGGCGACGAGTATATGTACGGCGGAGGCTGCGCCGAGAC
TGACGAGGATCATGCCCATTGGCTGGAAGCCAGAATGCTGCTGGACAACA
TATACCTGCAGGACGGCCTGATCGCCTCCCTGTACAGACCCGAGGCTGAC
AAAGTGGCTGCCATCGAGGGCGAGTTCAAGCTGAGGACCGAGCAGAGAAA
GACATTTGTGGAACTGATGAAGCGGGGCGACCTGCCTGTGTGGCTGGCCT
ATCAGGTGGCATCTGCCGGCATCACCTACACCGACAGACGGTGGTGCTTC
GACGGCACCACCAACAACACCATCATGGAAGATAGCGTGCCAGCCGAAGT
GTGGACCAAATACGGCGAGAAGCGCGTGCTGAAGCCCCGGTGGATGGACG
CCAGAGTGTGTTCTGATCACGCCGCACTGAAGTCCTTCAAAGAGTTCGCC
GCTGGCAAGTGATGAGCGGCCGCTCGAGTACGTCTG
Protein sequence of CprME/NS
(SEQ ID NO: 19)
MKNPKKKSGGFRIVNMLKRGVARVNPLGGLKRLP
AGLLLGHGPIRMVLAILAFLRFTAIKPSLGLINRWGSVGKKEAMEIIKKF
KKDLAAMLRIINARKERKRRGADTSIGilGLLLTTAMAAEITRRGSAYYM
YLDRSDAGKAISFATTLGVNKCHVQIMDLGHMCDATMSYECPMLDEGVEP
DDVDCWCNTTSTWVVYGTCHHKKGEARRSRRAVTLPSHSTRKLQTRSQTW
LESREYTKHLIKVENWIFRNPGFALAAVAIAWLLGSSTSQKVIYLVMILL
IAPAYSIRCIGVSNRDFVEGMSGGTWVDVVLEHGGCVTVMAQDKPTVDIE
LVTTTVSNMAEVRSYCYEASISDMASDSRCPTQGEAYLDKQSDTQYVCKR
TLVDRGWGNGCGLFGKGSLVTCAKFTCSKKMTGKSIQPENLEYRIMLSVH
GSQHSGMIVNDIGHETDENRAKVEVTPNSPRAEATLGGFGSLGLDCEPRT
GLDFSDLYYLTMNNKHWLVHKEWFHDIPLPWHAGADTGTPHWNNKEALVE
FKDAHAKRQTVVVLGSQEGAVHTALAGALEAEMDGAKGRLFSGHLKCRLK
MDKLRLKGVSYSLCTAAFTFTKVPAETLHGTVTVEVQYAGTDGPCKVPAQ
MAVDMQTLTPVGRLITANPVITESTENSKMMLELDPPFGDSYIVIGVGDK
KITHHWHRSGSTIGKAFEATVRGAKRMAVLGDTAWDEGSVGGVENSLGKG
IHQIFGAAFKSLEGGMSWESQILIGTLLVWLGLNTKNGSISLTCLALGGV
MIFLSTAVSAPSEVLTAVGLICALAGGFAKADIEMAGPMAAVGLLIVSYV
VSGKSVDMYIERAGDITWEKDAEVTGNSPRLDVALDESGDFSLVEEDGPP
MREIILKVVLMAICGMNPTATPFAAGAWYVYVKTGKRSGALWDVPAPKEV
KKGETTDGVYRVMTRRLLGSTQVGVGVMQEGVFHTMWHVTKGAALRSGEG
RLDPYWGDVKQDLVSYCGPWKLDAAWDGLSEVQLLAVPPGERARNIQTLP
GIFKTKDGDIGAVALDYPAGTSGSP1LDKCGRVIGLYGNGVVIKNGSYVS
AITQGKREEETPVECFEPSMLKKKQLTVLDLHPGAGKTRRVLPEIVREAI
KKRLRTVILAPTRVVAAEMEEALRGLPVRYMTTAVNVTHSGTEIVDLMCH
ATFTSRLLQPIRVPNYNLYIMDEAHFTDPSSIAARGYISTRVEMGEAAAI
FMTATPPGTRDAFPDSNSPIMDTEVEVPERAWSSGFDWVTDHSGKTVWFV
PSVRNGNEIAACLTKAGKRVIQLSRKTFETEFQKTKNQEWDFVITTDISE
MGANFKADRVIDSRRCLKPVILDGERVILAGPMPVTHASAAQRRGRIGRN
PNKPGDEYMYGGGCAETDEDHAHWLEARMLLDNIYLQDGLIASLYRPEAD
KVAAIEGEFKLRTEQRKTFVELMKRGDLPVWLAYQVASAGITYTDRRWCF
DGTTNNTIMEDSVPAEVWTKYGEKRVLKPRWMDARVCSDHAALKSFKEFA
AGK

### Example 2

### Antigen Combination in Single Viral Vectors

The aim of this study is provide a new bivalent vaccine to induce simultaneous immunity against Zika and Chikungunya, simplifying future vaccination campaigns for countries where both diseases co-circulate in the same regions, and where protection against both diseases is needed. Infection by ZIKV is a major concern worldwide due to the neurologic conditions, such as Guillain-Barré syndrome and a concurrent 20-fold increase in the incidence of microcephaly during the ZIKV outbreak in Brazil between 2014 and 2015 and in Mexico, where microcephaly caused by ZIKV has been confirmed. Aedes mosquitoes transmit Chikungunya virus

(CHIKV), ZIKV and Dengue in the same geographical regions. CHIKV produces symptomatic disease in approximately ¾ of infected people, leading in many cases to long-term sequelae in people of all ages. Persistent arthritis cause disability for several years, contributing to poverty as young adults are unable to perform their physical activities required for work. Costs for families and governments are augmented due to the need to administer anti-inflammatory drugs to provide a short-term relief in patients. No vaccine is yet licensed for the prevention of CHIKV or ZIKV infections.

The sudden presence of Zika and Chikungunya in the same geographical regions have overwhelmed health systems that were already challenged by Dengue, thus increasing the failure to provide treatment and preventive measures to their populations during the outbreak, while posing new challenges for treatment of both Zika and Chikungunya due to the long-term sequelae of more than 6 years for these diseases. These diseases are transitioning from an epidemic nature towards endemic diseases due to enabling drivers such as poor socioeconomic conditions, climate change and migration. A major breakthrough will be to provide governments with tools to simultaneously fight these highly prevalent arbovirus diseases and a multivalent vaccine able to protect against both Zika and Chikungunya would be an ideal preventive solution. This proposal has various aims:

Provided is a bivalent vaccine to provide simultaneous protection against Zika and Chikungunya, caused by two arboviruses co-circulating in the same geographical regions. Both, Zika and Chikunguna vaccines will be applied concurrently in a single administration without the need of adjuvants, taking into advantage that they are based on the same ChAdOx1 platform. This approach is simple and has the potential to stimulate fast induction of antibodies in only 10 days after the administration to provide long-lasting immunity in humans.

A multi-valent vaccine to protect against Zika and Chikungunya viruses can be highly attractive for vaccination campaigns in regions where both viruses co-circulate. This would be an efficient strategy to reduce costs and prevent arbovirus diseases that would rely on a concurrent delivery of the multivalent vaccine.

For a number of years, approaches have been pursued to develop viral vectors expressing multiple antigens to provide better protection against infection by increasing the breadth of both, T-cell and antibody responses to multiple antigens (Ported, D. W. et al. Vaccine 2011; Prieur, D. et al. PNAS, 2013; Bauza, K et al; Inf and Immun, 2016). Nevertheless, performance of the vaccine upon a challenge is difficult to predict, both in mice and human challenges with pathogens. Porter et al.

reported two poxviral vectors expressing various malaria vaccine candidates. The polyprotein vaccine insert known as L3SEPTL contained pre-erythrocytic malaria vaccine antigens linked together, including liver stage antigen 3 (LSA3), sporozoite threonine and asparagine rich protein (STARP), exported protein-1 (Exp1), Pfs16, thrombospondin-related adhesion protein (TRAP) and liver stage antigen-1 (LSA1). Surprisingly, T-cell immunogenicity against the antigens in the L3SEPTL vaccine was lower than viral vectors expressing individually some of the antigens. Protection against a challenge was negative and the vaccine was not further developed.

Bauza et al. (Inf and Immun, 2016) reported that a combination of the vaccine candidates Circumsporozoite Protein (CSP) and Thrombospondin Related Anonymous Protein (TRAP) from Plasmodium berghei failed to significantly enhance protective efficacy when expressed by viral vectors, but this was improved when CSP was used as a protein and TRAP as a viral vector. Similar observations were made by Salman et al. (Sci. Reports, 2017) when assessing a chimeric P. vivax CSP antigen expressed by chimpanzee adenoviruses, whereby protective efficacy against a sporozoite challenge was low compared to the antigens presented in Rv21, a virus-like particle currently developed for clinical trials.

Nevertheless, the present invention finds that a combination of viral vectors expressing the structural proteins of the Chikungunya and Zika viruses could have a potential to induce strong antibody responses, at least similar to responses elicited by individual viral vectors.

### Chikungunya and Zika Viruses Bivalent Formulation

To determine if both vaccines can be injected as a bivalent formulation without compromising immunogenicity, a combination of the ChAdOx1-Zika with the ChAdOx1-Chikungunya vaccines was administered into mice, either as a mixed single component or co-administered in different legs (FIG. 10). Results indicated that immune responses against Zika and Chikungunya proteins were similar (no statistical differences) when vaccines were administered alone or combined. These preliminary results support their use as a bivalent vaccine.

An analysis using a single time point may not reflect if memory responses, the goal of vaccination, are sustained at high levels and therefore, antibody responses in mice were assessed at various time points to investigate if the kinetics of the antibody responses is affected positively or negatively by a vaccine combination. Surprisingly, it was observed that a mixture in the same syringe of the two vaccines or a co-vaccination in different legs induced similar antibody responses to those induced individually by a ChAdOx1-Chikungunya vaccine or a ChAdOx1-Zika for over 20 weeks after a single vaccination or one week post MVA boost (FIGS. 11 and 12)

### Chikungunya Vaccine Sequences

### Structural Genes Encoded by ChAdOx1 Spol (ChAdOx1 Chik) Vaccine

NUCLEOTIDE SEQUENCE
(SEQ ID NO: 14)
ATGGAATTCATCCCCACCCAGACCTTCTACAACCGCAGATACCAGCCCAG
ACCCTGGACCCCCAGACCCACCATCCAAGTGATCAGACCCCGGCCTAGAC
CCCAGAGACAGGCTGGACAGCTGGCTCAGCTGATCTCCGCCGTGAACAAG
CTGACCATGAGAGCCGTGCCCCAGCAGAAGCCCAGAAAGAACCGGAAGAA
CAAGAAGCAGAAACAGAAGCAGCAGGCCCCCCAGAACGACCCCAAGCAGA
AGAAGCAGCCTCCTCAGAAGAAACCCGCCCAGAAGAAGAAAAAGCCCGGC
AGACGCGAGCGGATGTGCATGAAGATCGAGAACGACTGCATCTTCGAAGT
GAAGCACGAGGGCAAAGTGATGGGCTACGCCTGCCTCGTGGGCGACAAAG
TGATGAAGCCCGCCCACGTGAAGGGCACCATCGACAATGCCGACCTGGCC
AAGCTGGCCTTCAAGCGGAGCAGCAAATACGACCTGGAATGCGCCCAGAT
CCCCGTGCACATGAAGTCCGACGCCAGCAAGTTCACCCACGAGAAGCCCG
AGGGCTACTACAACTGGCACCATGGCGCCGTGCAGTACAGCGGCGGCAGA
TTCACAATCCCCACCGGCGCTGGAAAGCCTGGCGATAGCGGCAGACCCAT
CTTCGACAACAAGGGCCGGGTGGTGGCCATCGTGCTGGGCGGAGCTAATG
AGGGCGCCAGAACAGCCCTGAGCGTCGTGACCTGGAACAAGGACATCGTG
ACCAAGATCACCCCCGAGGGCGCCGAGGAATGGTCCCTGGCTATCCCTGT
GATGTGCCTGCTGGCCAACACCACCTTCCCATGCAGCCAGCCCCCTTGCA
CCCCTTGCTGCTACGAGAAAGAGCCCGAGAGCACCCTGCGGATGCTGGAA
GATAACGTGATGAGGCCCGGCTACTACCAGCTGCTGAAGGCCTCCCTGAC
CTGCAGCCCTCACCGGCAGAGAAGATCCACCAAGGACAACTTCAACGTGT
ACAAGGCCACCAGACCCTACCTGGCCCACTGCCCTGATTGTGGCGAGGGC
CACTCTTGCCACTCTCCCGTGGCCCTGGAACGGATCAGAAACGAGGCCAC
CGACGGCACCCTGAAGATCCAGGTGTCCCTGCAGATCGGCATCAAGACCG
ACGACAGCCACGACTGGACCAAGCTGCGGTACATGGACAACCACATGCCC
GCCGATGCCGAGAGGGCAGGACTGCTCGTGCGGACATCTGCCCCCTGTAC
CATCACCGGCACAATGGGCCACTTCATCCTGGCCAGATGCCCCAAGGGCG
AGACACTGACCGTGGGCTTCACCGATGGCCGGAAGATCAGCCACAGCTGC
ACCCACCCCTTCCACCACGATCCTCCCGTGATCGGCAGAGAGAAGTTCCA
CAGCAGACCCCAGCACGGCAAAGAGCTGCCCTGCAGCACATACGTGCAGA
GCACAGCCGCCACCGCCGAAGAGATCGAGGTGCACATGCCTCCCGACACC
CCCGACAGAACCCTGATGTCTCAGCAGAGCGGCAACGTGAAGATCACCGT
GAACGGCCAGACCGTGCGGTACAAGTGCAACTGCGGCGGCTCCAATGAGG
GCCTGACCACCACAGACAAAGTGATCAACAACTGCAAGATCGACCAGTGC
CACGCCGCCGTGACCAACCACAAGAAGTGGCAGTACAACAGCCCCCTGGT
GCCCAGAAATGCCGAGCTGGGCGACCGGAAGGGCAAGATCCACATCCCTT
TCCCCCTGGCCAACGTGACCTGCCGGGTGCCCAAAGCCAGAAACCCCACC
GTGACCTACGGCAAGAACCAAGTGATTATGCTGCTGTACCCCGACCACCC
CACCCTGCTGAGCTACAGAAACATGGGCGAGGAACCCAACTACCACGAAG
AGTGGGTCACCCACAAGAAAGAAGTGCGGCTGACCGTGCCCACCGAGGGC
CTGGAAGTGACCTGGGGCAACAACGAGCCCTACAAGTACTGGCCCCAGCT
GAGCACCAATGGCACAGCCCACGGACACCCCCACGAGATCATCCTGTACT
ACTACGAGCTGTACCCTACCATGACCGTCGTGATCGTGTCTGTGGCCAGC
TTCGTGCTGCTGAGCATGGTGGGAACAGCCGTGGGCATGTGTATGTGCGC
CAGACGGCGGTGCATCACCCCTTACGAACTGACCCCTGGCGCCACCGTGC
CCTTTCTGCTGAGCCTGATCTGCTGCATCCGGACCGCCAAGGCCGCCACC
TATTATGAGGCCGCTGCCTACCTGTGGAACGAGCAGCAGCCCCTGTTTTG
GCTGCAAGCCCTGATTCCTCTGGCCGCCCTGATCGTGCTGTGCAACTGCC
TGAGACTGCTGCCCTGCTGCTGCAAGACCCTGGCCTTTCTGGCCGTGATG
AGCATCGGAGCCCACACCGTGTCTGCCTACGAGCACGTGACCGTGATCCC
CAACACAGTGGGCGTGCCCTACAAAACCCTCGTGAACAGACCCGGCTACA
GCCCTATGGTGCTGGAAATGGAACTGCTGAGCGTGACCCTGGAACCCACC
CTGAGCCTGGACTACATCACATGCGAGTACAAGACAGTGATCCCTAGCCC
CTACGTGAAGTGCTGCGGCACCGCCGAGTGCAAGGACAAGAGCCTGCCCG
ACTACAGCTGCAAGGTGTTCACCGGCGTGTACCCCTTCATGTGGGGCGGA
GCCTACTGCTTTTGCGACGCCGAGAACACACAGCTGAGCGAGGCCCACGT
GGAAAAGAGCGAGAGCTGCAAAACCGAGTTCGCCAGCGCCTACAGGGCCC
ACACAGCCTCTGCCTCTGCCAAGCTGAGAGTGCTGTACCAGGGCAACAAT
ATCACCGTGGCCGCCTACGCCAACGGCGACCATGCCGTGACAGTGAAGGA
CGCCAAGTTCATCGTGGGCCCCATGAGCAGCGCCTGGACACCCTTCGATA
ACAAGATTGTGGTGTATAAGGGGGATGTGTACAACATGGACTACCCCCCC
TTTGGCGCCGGACGGCCTGGACAGTTTGGCGACATCCAGAGCAGAACCCC
TGAGAGCAAGGACGTGTACGCCAACACCCAGCTGGTGCTGCAGAGGCCTG
CAGCCGGAACAGTGCACGTGCCATACTCTCAGGCCCCCAGCGGCTTCAAG
TATTGGCTGAAAGAGAGAGGCGCCAGCCTGCAGCATACCGCCCCTTTCGG
CTGTCAGATCGCCACCAATCCTGTGCGGGCCGTGAATTGCGCCGTGGGAA
ACATCCCCATCAGCATCGACATCCCCGACGCCGCCTTCACCAGAGTGGTG
GATGCCCCTAGCCTGACCGACATGAGCTGCGAAGTGCCCGCCTGCACACA
CAGCAGCGATTTTGGCGGAGTGGCCATCATTAAGTACGCCGCCTCCAAGA
AAGGCAAGTGTGCCGTGCACAGCATGACCAACGCCGTGACAATCCGCGAG
GCCGAGATTGAGGTGGAAGGCAACAGCCAGCTGCAGATCAGCTTCTCCAC
AGCCCTGGCCAGCGCCGAGTTCAGAGTGCAAGTGTGCAGCACCCAGGTGC
ACTGCGCTGCCGCTTGTCACCCCCCCAAGGACCACATCGTGAACTACCCT
GCCAGCCACACCACCCTGGGCGTGCAGGATATCAGCACCACCGCCATGTC
CTGGGTGCAGAAAATCACAGGGGGCGTGGGACTGATCGTGGCCGTGGCTG
CTCTGATTCTGATTGTGGTGCTGTGCGTGTCCTTCAGCCGGCACTGATGA
PROTEIN SEQUENCE (Structural Polyprotein)
(SEQ ID NO: 15)
MEFIPTQTFYNRRYQPRPWTPRPTIQVIRPRPRPQRQAGQLAQLISAVNK
LTMRAVPQQKPRKNRKNKKQKQKQQAPQNDPKQKKQPPQKKPAQKKKKPG
RRERMCMKIENDCIFEVKHEGKVMGYACLVGDKVMKPAHVKGTIDNADLA
KLAFKRSSKYDLECAQIPVHMKSDASKFTHEKPEGYYNWHHGAVQYSGGR
FTIPTGAGKPGDSGRPIFDNKGRVVAIVLGGANEGARTALSVVTWNKDIV
TKITPEGAEEWSLAIPVMCLLANTTFPCSQPPCTPCCYEKEPESTLRMLE
DNVMRPGYYQLLKASLTCSPHRQRRSTKDNFNVYKATRPYLAHCPDCGEG
HSCHSPVALERIRNEATDGTLKIQVSLQIGIKTDDSHDWTKLRYMDNHMP
ADAERAGLLVRTSAPCTITGTMGHFILARCPKGETLTVGFTDGRKISHSC
THPFHHDPPVIGREKFHSRPQHGKELPCSTYVQSTAATAEEIEVHMPPDT
PDRTLMSQQSGNVKITVNGQTVRYKCNCGGSNEGLTTTDKVINNCKIDQC
HAAVTNHKKWQYNSPLVPRNAELGDRKGKIHIPFPLANVTCRVPKARNPT
VTYGKNQVIMLLYPDHPTLLSYRNMGEEPNYHEEWVTHKKEVRLTVPTEG
LEVTWGNNEPYKYWPQLSTNGTAHGHPHEIILYYYELYPTMTVVIVSVAS
FVLLSMVGTAVGMCMCARRRCITPYELTPGATVPFLLSLICCIRTAKAAT
YYEAAAYLWNEQQPLFWLQALIPLAALIVLCNCLRLLPCCCKTLAFLAVM
SIGAHTVSAYEHVTVIPNTVGVPYKTLVNRPGYSPMVLEMELLSVTLEPT
LSLDYITCEYKTVIPSPYVKCCGTAECKDKSLPDYSCKVFTGVYPFMWGG
AYCFCDAENTQLSEAHVEKSESCKTEFASAYRAHTASASAKLRVLYQGNN
ITVAAYANGDHAVTVKDAKFIVGPMSSAWTPFDNKIVVYKGDVYNMDYPP
FGAGRPGQFGDIQSRTPESKDVYANTQLVLQRPAAGTVHVPYSQAPSGFK
YWLKERGASLQHTAPFGCQIATNPVRAVNCAVGNIPISIDIPDAAFTRVV
DAPSLTDMSCEVPACTHSSDFGGVAIIKYAASKKGKCAVHSMTNAVTIRE
AEIEVEGNSQLQISFSTALASAEFRVQVCSTQVHCAAACHPPKDHIVNYP
ASHTTLGVQDISTTAMSWVQKITGGVGLIVAVAALILIVVLCVSFSRH**

### Structural Genes Encoded by ChAdOx1 Chik-Non Capsid Vaccine

NUCLEOTIDE SEQUENCE
(SEQ ID NO: 16)
GAGGAATGGTCCCTGGCTATCCCTGTGATGTGCCTGCTGGCCAACACCAC
CTTCCCATGCAGCCAGCCCCCTTGCACCCCTTGCTGCTACGAGAAAGAGC
CCGAGAGCACCCTGCGGATGCTGGAAGATAACGTGATGAGGCCCGGCTAC
TACCAGCTGCTGAAGGCCTCCCTGACCTGCAGCCCTCACCGGCAGAGAAG
ATCCACCAAGGACAACTTCAACGTGTACAAGGCCACCAGACCCTACCTGG
CCCACTGCCCTGATTGTGGCGAGGGCCACTCTTGCCACTCTCCCGTGGCC
CTGGAACGGATCAGAAACGAGGCCACCGACGGCACCCTGAAGATCCAGGT
GTCCCTGCAGATCGGCATCAAGACCGACGACAGCCACGACTGGACCAAGC
TGCGGTACATGGACAACCACATGCCCGCCGATGCCGAGAGGGCAGGACTG
CTCGTGCGGACATCTGCCCCCTGTACCATCACCGGCACAATGGGCCACTT
CATCCTGGCCAGATGCCCCAAGGGCGAGACACTGACCGTGGGCTTCACCG
ATGGCCGGAAGATCAGCCACAGCTGCACCCACCCCTTCCACCACGATCCT
CCCGTGATCGGCAGAGAGAAGTTCCACAGCAGACCCCAGCACGGCAAAGA
GCTGCCCTGCAGCACATACGTGCAGAGCACAGCCGCCACCGCCGAAGAGA
TCGAGGTGCACATGCCTCCCGACACCCCCGACAGAACCCTGATGTCTCAG
CAGAGCGGCAACGTGAAGATCACCGTGAACGGCCAGACCGTGCGGTACAA
GTGCAACTGCGGCGGCTCCAATGAGGGCCTGACCACCACAGACAAAGTGA
TCAACAACTGCAAGATCGACCAGTGCCACGCCGCCGTGACCAACCACAAG
AAGTGGCAGTACAACAGCCCCCTGGTGCCCAGAAATGCCGAGCTGGGCGA
CCGGAAGGGCAAGATCCACATCCCTTTCCCCCTGGCCAACGTGACCTGCC
GGGTGCCCAAAGCCAGAAACCCCACCGTGACCTACGGCAAGAACCAAGTG
ATTATGCTGCTGTACCCCGACCACCCCACCCTGCTGAGCTACAGAAACAT
GGGCGAGGAACCCAACTACCACGAAGAGTGGGTCACCCACAAGAAAGAAG
TGCGGCTGACCGTGCCCACCGAGGGCCTGGAAGTGACCTGGGGCAACAAC
GAGCCCTACAAGTACTGGCCCCAGCTGAGCACCAATGGCACAGCCCACGG
ACACCCCCACGAGATCATCCTGTACTACTACGAGCTGTACCCTACCATGA
CCGTCGTGATCGTGTCTGTGGCCAGCTTCGTGCTGCTGAGCATGGTGGGA
ACAGCCGTGGGCATGTGTATGTGCGCCAGACGGCGGTGCATCACCCCTTA
CGAACTGACCCCTGGCGCCACCGTGCCCTTTCTGCTGAGCCTGATCTGCT
GCATCCGGACCGCCAAGGCCGCCACCTATTATGAGGCCGCTGCCTACCTG
TGGAACGAGCAGCAGCCCCTGTTTTGGCTGCAAGCCCTGATTCCTCTGGC
CGCCCTGATCGTGCTGTGCAACTGCCTGAGACTGCTGCCCTGCTGCTGCA
AGACCCTGGCCTTTCTGGCCGTGATGAGCATCGGAGCCCACACCGTGTCT
GCCTACGAGCACGTGACCGTGATCCCCAACACAGTGGGCGTGCCCTACAA
AACCCTCGTGAACAGACCCGGCTACAGCCCTATGGTGCTGGAAATGGAAC
TGCTGAGCGTGACCCTGGAACCCACCCTGAGCCTGGACTACATCACATGC
GAGTACAAGACAGTGATCCCTAGCCCCTACGTGAAGTGCTGCGGCACCGC
CGAGTGCAAGGACAAGAGCCTGCCCGACTACAGCTGCAAGGTGTTCACCG
GCGTGTACCCCTTCATGTGGGGCGGAGCCTACTGCTTTTGCGACGCCGAG
AACACACAGCTGAGCGAGGCCCACGTGGAAAAGAGCGAGAGCTGCAAAAC
CGAGTTCGCCAGCGCCTACAGGGCCCACACAGCCTCTGCCTCTGCCAAGC
TGAGAGTGCTGTACCAGGGCAACAATATCACCGTGGCCGCCTACGCCAAC
GGCGACCATGCCGTGACAGTGAAGGACGCCAAGTTCATCGTGGGCCCCAT
GAGCAGCGCCTGGACACCCTTCGATAACAAGATTGTGGTGTATAAGGGGG
ATGTGTACAACATGGACTACCCCCCCTTTGGCGCCGGACGGCCTGGACAG
TTTGGCGACATCCAGAGCAGAACCCCTGAGAGCAAGGACGTGTACGCCAA
CACCCAGCTGGTGCTGCAGAGGCCTGCAGCCGGAACAGTGCACGTGCCAT
ACTCTCAGGCCCCCAGCGGCTTCAAGTATTGGCTGAAAGAGAGAGGCGCC
AGCCTGCAGCATACCGCCCCTTTCGGCTGTCAGATCGCCACCAATCCTGT
GCGGGCCGTGAATTGCGCCGTGGGAAACATCCCCATCAGCATCGACATCC
CCGACGCCGCCTTCACCAGAGTGGTGGATGCCCCTAGCCTGACCGACATG
AGCTGCGAAGTGCCCGCCTGCACACACAGCAGCGATTTTGGCGGAGTGGC
CATCATTAAGTACGCCGCCTCCAAGAAAGGCAAGTGTGCCGTGCACAGCA
TGACCAACGCCGTGACAATCCGCGAGGCCGAGATTGAGGTGGAAGGCAAC
AGCCAGCTGCAGATCAGCTTCTCCACAGCCCTGGCCAGCGCCGAGTTCAG
AGTGCAAGTGTGCAGCACCCAGGTGCACTGCGCTGCCGCTTGTCACCCCC
CCAAGGACCACATCGTGAACTACCCTGCCAGCCACACCACCCTGGGCGTG
CAGGATATCAGCACCACCGCCATGTCCTGGGTGCAGAAAATCACAGGGGG
CGTGGGACTGATCGTGGCCGTGGCTGCTCTGATTCTGATTGTGGTGCTGT
GCGTGTCCTTCAGCCGGCACTGATGA
PROTEIN SEQUENCE (Structural Polyprotein with no
Capsid included)
(SEQ ID NO: 17)
MEEWSLAIPVMCLLANTTFPCSQPPCTPCCYEKEPESTLRMLEDNVMRPG
YYQLLKASLTCSPHRQRRSTKDNFNVYKATRPYLAHCPDCGEGHSCHSPV
ALERIRNEATDGTLKIQVSLQIGIKTDDSHDWTKLRYMDNHMPADAERAG
LLVRTSAPCTITGTMGHFILARCPKGETLTVGFTDGRKISHSCTHPFHHD
PPVIGREKFHSRPQHGKELPCSTYVQSTAATAEEIEVHMPPDTPDRTLMS
QQSGNVKITVNGQTVRYKCNCGGSNEGLTTTDKVINNCKIDQCHAAVTNH
KKWQYNSPLVPRNAELGDRKGKIHIPFPLANVTCRVPKARNPTVTYGKNQ
VIMLLYPDHPTLLSYRNMGEEPNYHEEWVTHKKEVRLTVPTEGLEVTWGN
NEPYKYWPQLSTNGTAHGHPHEIILYYYELYPTMTVVIVSVASFVLLSMV
GTAVGMCMCARRRCITPYELTPGATVPFLLSLICCIRTAKAATYYEAAAY
LWNEQQPLFWLQALIPLAALIVLCNCLRLLPCCCKTLAFLAVMSIGAHTV
SAYEHVTVIPNTVGVPYKTLVNRPGYSPMVLEMELLSVTLEPTLSLDYIT
CEYKTVIPSPYVKCCGTAECKDKSLPDYSCKVFTGVYPFMWGGAYCFCDA
ENTQLSEAHVEKSESCKTEFASAYRAHTASASAKLRVLYQGNNITVAAYA
NGDHAVTVKDAKFIVGPMSSAWTPFDNKIVVYKGDVYNMDYPPFGAGRPG
QFGDIQSRTPESKDVYANTQLVLQRPAAGTVHVPYSQAPSGFKYWLKERG
ASLQHTAPFGCQIATNPVRAVNCAVGNIPISIDIPDAAFTRVVDAPSLTD
MSCEVPACTHSSDFGGVAIIKYAASKKGKCAVHSMTNAVTIREAEIEVEG
NSQLQISFSTALASAEFRVQVCSTQVHCAAACHPPKDHIVNYPASHTTLG
VQDISTTAMSWVQKITGGVGLIVAVAALILIVVLCVSFSRH**

1. A Zika viral vector vaccine comprising nucleic acid encoding a Zika virus structural antigen, wherein the nucleic acid encoding a Zika virus structural antigen comprises a sequence encoding Zika virus envelope DIII, or part thereof.

2. The Zika viral vector vaccine according to claim 1, wherein the nucleic acid encoding the Zika virus structural antigen comprises or consists of the sequence of SEQ ID NO: 7, or part(s) thereof.

3. The Zika viral vector vaccine according to claim 1, wherein the nucleic acid encoding the Zika virus structural antigen comprises or consists of the sequence of SEQ ID NO: 9, or part(s) thereof.

4. The Zika viral vector vaccine according to any preceding claim, wherein the nucleic acid encoding the Zika virus structural antigen comprises or consists of the sequence of SEQ ID NO: 1.

5. The Zika viral vector vaccine according to any preceding claim, wherein the Zika virus envelope comprises the whole Zika virus envelope DIII sequence; or
the Zika virus envelope DIII comprises the whole Zika virus envelope DIII sequence of SEQ ID NO: 1.

6. The Zika viral vector vaccine according to any preceding claim, wherein the Zika virus envelope DIII or the nucleic acid encoding the Zika virus envelope DIII is a natural or modified variant thereof.

7. The Zika viral vector vaccine according to any preceding claim, wherein variants of the nucleic acid encoding the Zika virus envelope DIII comprise or consist of a sequence having at least 80% identity with SEQ ID NO: 1.

8. The Zika viral vector vaccine according to any preceding claim, wherein the sequence identity is over at least 50 consecutive nucleotides of SEQ ID NO: 1.

9. The Zika viral vector vaccine according to any preceding claim, wherein variants of Zika virus envelope DIII comprise or consist of a truncated sequence of the Zika virus envelope DIII encoding sequence of SEQ ID NO: 1.

10. The Zika viral vector vaccine according to any preceding claim, wherein the Zika viral vector vaccine does not comprise sequence encoding Zika virus TM (transmembrane) domain or part thereof.

11. The Zika viral vector vaccine according to any preceding claim, wherein the Zika viral vector vaccine does not comprise sequence encoding Zika virus prM domain or part thereof.

12. The Zika viral vector vaccine according to any preceding claim, wherein the Zika viral vector vaccine does not comprise sequence encoding a Zika virus non-structural domain or part(s) thereof.

13. The Zika viral vector vaccine according to any preceding claim, wherein the Zika viral vector vaccine comprises sequence encoding a Zika virus non-structural domain or part(s) thereof.

14. A Zika viral vector vaccine comprising nucleic acid encoding a Zika virus structural antigen, wherein the nucleic acid encoding a Zika virus structural antigen comprises a sequence encoding at least part of the Zika virus prM, and a sequence encoding at least part of the Zika virus envelope protein.

15. The Zika viral vector vaccine according to claim 14, wherein the nucleic acid encoding a Zika virus structural antigen consists essentially of a sequence encoding Zika virus envelope, or a part thereof, and prM, or part thereof.

16. The Zika viral vector vaccine according to claim 14 or 15, wherein the nucleic acid encoding the Zika virus envelope comprises the sequence of SEQ ID NO: 3 (ZENV_noTM), or a part thereof.

17. The Zika viral vector vaccine according to any of claims 14 to 16, wherein the nucleic acid encoding the Zika virus structural antigen comprises or consists of the sequence of SEQ ID NO: 7, or part(s) thereof.

18. The Zika viral vector vaccine according to any of claims 14 to 17, wherein the nucleic acid encoding the Zika virus structural antigen consists of the sequence of SEQ ID NO: 7, or part(s) thereof.

19. The Zika viral vector vaccine according to any of claims 14 to 18, wherein the Zika virus envelope comprises the whole envelope sequence.

20. The Zika viral vector vaccine according to any of claims 14 to 18, wherein the Zika virus envelope comprises at least two of the DI, DII or DII domains, or parts thereof, of the envelope sequence.

21. The Zika viral vector vaccine according to any of claims 14 to 18, wherein the Zika virus envelope may comprise at part of all DI, DII and DII domains of the envelope sequence.

22. The Zika viral vector vaccine according to any of claims 14 to 21, wherein the Zika virus envelope is a natural or modified variant thereof; or
the nucleic acid encoding the Zika virus envelope may be a natural or modified variant thereof.

23. The Zika viral vector vaccine according to any of claims 14 to 22, wherein variants of the nucleic acid encoding the Zika virus envelope comprise or consist of a sequence having at least 80% identity with SEQ ID NO: 3.

24. The Zika viral vector vaccine according to claim 23, wherein the sequence identity is over at least 50 consecutive nucleotides of SEQ ID NO: 3.

25. The Zika viral vector vaccine according to any of claims 14 to 24, wherein variants of Zika virus envelope comprise or consist of a truncated sequence of the Zika virus envelope sequence of SEQ ID NO: 3.

26. The Zika viral vector vaccine according to any of claims 14 to 25, wherein the Zika virus prM comprises the whole prM sequence; or
the Zika virus prM comprises a sequence encoding the whole prM sequence of SEQ ID NO: 13.

27. The Zika viral vector vaccine according to any of claims 14 to 26, wherein the Zika virus prM is a natural or modified variant thereof; or
the nucleic acid encoding the Zika virus prM is a natural or modified variant thereof.

28. The Zika viral vector vaccine according to any of claims 14 to 27, wherein variants of the nucleic acid encoding the Zika virus prM comprise or consist of a sequence having at least 80% identity with SEQ ID NO: 13.

29. The Zika viral vector vaccine according to claim 28, wherein the sequence identity is over at least 50 consecutive nucleotides of SEQ ID NO: 13.

30. The Zika viral vector vaccine according to any of claims 14 to 27, wherein variants of Zika virus prM comprise or consist of a truncated sequence encoding the Zika virus prM sequence of SEQ ID NO: 13.

31. The Zika viral vector vaccine according to any of claims 14 to 30, wherein the Zika viral vector vaccine does not comprise sequence encoding Zika virus TM (transmembrane) domain or part thereof.

32. The Zika viral vector vaccine according to any of claims 14 to 31, wherein the Zika viral vector vaccine does not comprise sequence encoding a Zika virus non-structural domain or part(s) thereof.

33. The Zika viral vector vaccine according to any of claims 14 to 31, wherein the Zika viral vector vaccine comprises sequence encoding a Zika virus non-structural domain or part(s) thereof.

34. The Zika viral vector vaccine according to any preceding claim, wherein the Zika viral vector vaccine further encodes a peptide adjuvant, such as a TPA (tissue plasminogen activator) sequence, or a functional variant thereof.

35. The Zika viral vector vaccine according to any preceding claim, wherein the viral vector comprises nucleic acid encoding non-Zika viral protein, such as adenovirus protein(s) or MVA protein(s).

36. The Zika viral vector vaccine according to any preceding claim, wherein the Zika virus structural antigen is expressed as a non-secreting protein in the cell.

37. The Zika viral vector vaccine according to any preceding claim in combination with another therapeutically or prophylactically active ingredient.

38. The Zika viral vector vaccine according to claim 37, wherein the therapeutically or prophylactically active ingredient comprises a Chikungunya vaccine, optionally wherein the Chikungunya vaccine is a Chikungunya viral vector vaccine.

39. The Zika viral vector vaccine according to claim 38, wherein the Chikungunya viral vector vaccine comprises nucleic acid of SEQ ID NO: 14 or SEQ ID NO: 16; or wherein the Chikungunya viral vector vaccine comprises nucleic acid encoding polypeptides of SEQ ID NO: 15 or SEQ ID NO: 17.

40. The Zika viral vector vaccine according to any preceding claim, wherein the Zika viral vector vaccine is provided in a pharmaceutically acceptable carrier.

41. A nucleic acid encoding the Zika viral vector vaccine according to any preceding claim, or parts thereof.

42. A composition comprising the nucleic acid according to claim 40 or the viral vector according to any of claims 1 to 36.

43. The composition according to claim 42, wherein the composition further comprises another therapeutically or prophylactically active ingredient.

44. The composition according to claim 42 or 43, wherein the composition further comprises a Chikungunya vaccine, optionally wherein the Chikungunya vaccine is a Chikungunya viral vector vaccine.

45. The composition according to claim 44, wherein the Chikungunya viral vector vaccine comprises nucleic acid of SEQ ID NO: 14 or SEQ ID NO: 16; or wherein the Chikungunya viral vector vaccine comprises nucleic acid encoding polypeptides of SEQ ID NO: 15 or SEQ ID NO: 17.

46. A method of treatment or prophylaxis of Zika viral infection comprising the administration to a subject of:
the nucleic acid according to claim 41;
the composition according to any of claims 42 to 45; or
the viral vector vaccine according to any of claims 1 to 40.

47. A method of treatment or prophylaxis of Zika and/or Chikungunya viral infection comprising the administration to a subject of:
the composition according to any of claim 44 or 45; or
the viral vector vaccine according to any of claims 38 to 40.

48. An agent for use in the prophylaxis or treatment of Zika viral infection in a subject, the agent comprising or consisting of:
the nucleic acid according to claim 41;
the composition according to any of claims 42 to 45; or
the viral vector vaccine according to any of claims 1 to 40.

49. An agent for use in the prophylaxis or treatment of Zika and/or Chikungunya viral infection in a subject, the agent comprising or consisting of:
the composition according to any of claim 44 or 45; or
the viral vector vaccine according to any of claims 38 to 40.

50. The nucleic acid according to claim 41; the composition according to any one of claims 42 to 45; or the viral vector vaccine according to any of claims 1 to 40; for use in, or as, a vaccine.

51. A prime boost vaccination kit comprising
a prime vaccination comprising:
the nucleic acid according to claim 41;
the composition according to any one of claims 42 to 45; or
the viral vector according to any of claims 1 to 40; and optionally
a boost vaccination comprising:
the nucleic acid according to claim 41;
the composition according to any one of claims 42 to 45; or
the viral vector according to any of claims 1 to 40.

52. The kit according to claim 51, wherein the prime and boost vaccinations are different.

53. The kit according to claim 52, wherein the prime and boost vaccination comprise different viral vectors from different viral species.

54. A combination vaccine composition comprising the Zika viral vector vaccine according to any one of claims 1 to 40 and a Chikungunya vaccine.

55. The combination vaccine composition according to claim 54, wherein the Chikungunya vaccine comprises or consists of a Chikungunya viral vector vaccine.

56. The combination vaccine composition according to claim 55, wherein the Chikungunya viral vector vaccine comprises nucleic acid of SEQ ID NO: 14 or SEQ ID NO: 16; or wherein the Chikungunya viral vector vaccine comprises nucleic acid encoding polypeptides of SEQ ID NO: 15 or SEQ ID NO: 17.


SEQUENCE LISTING (32 sequences)

SEQ ID NO: 1 - DNA, 351 bp, Artificial Sequence
  Zika virus
atgaagatggacaagctgcggctgaagggcgtgtcctacagcctgtgtaccgccgccttcaccttcaccaagatccccgccgagacactgcacggcaccgtgactgtggaagtgcagtacgccggcaccgacggcccttgtaaagtgcctgctcagatggccgtggatatgcagaccctgacccccgtgggcagactgatcaccgccaaccctgtgatcaccgagagcaccgagaacagcaagatgatgctggaactggacccccccttcggcgactcctacatcgtgatcggcgtgggagagaagaagatcacccaccactggcacagaagcggcagcaccatcggcaag

SEQ ID NO: 2 - protein, 117 aa, Artificial Sequence
  Zika virus
MKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGK

SEQ ID NO: 3 - DNA, 1323 bp, Artificial Sequence
  Zika virus fusion sequence
atgcggtgtatcggcgtgtccaaccgggacttcgtggaaggcatgagcggcggcacatgggtggacgtggtgctggaacatggcggctgcgtgacagtgatggcccaggacaagcccaccgtggacatcgagctcgtgaccaccaccgtgtccaatatggccgaagtgcggagctactgctacgaggccagcatcagcgacatggccagcgacagcagatgccctacacagggcgaggcctacctggataagcagtccgacacccagtacgtgtgcaagcggaccctggtggatagaggctggggcaatggctgcggcctgtttggcaagggcagcctcgtgacctgcgccaagttcgcctgcagcaagaagatgaccggcaagagcatccagcccgagaacctggaataccggatcatgctgagcgtgcacggcagccagcactccggcatgatcgtgaacgacaccggccacgagacagacgagaaccgggccaaggtggaaatcacccccaacagccctagagccgaggccaccctgggcggctttggatctctgggactggactgcgagcccagaaccggcctggacttcagcgacctgtactacctgaccatgaacaacaagcactggctggtgcacaaagagtggttccacgacatccccctgccctggcatgccggcgctgatacaggcacaccccactggaacaacaaagaggctctggtggaattcaaggacgcccacgccaagcggcagaccgtggtggtgctgggatctcaggaaggcgccgtgcatacagctctggctggcgccctggaagccgaaatggatggcgccaaaggcagactgtccagcggccacctgaagtgccggctgaagatggacaagctgcggctgaagggcgtgtcctacagcctgtgtaccgccgccttcaccttcaccaagatccccgccgagacactgcacggcaccgtgactgtggaagtgcagtacgccggcaccgacggcccttgtaaagtgcctgctcagatggccgtggatatgcagaccctgacccccgtgggcagactgatcaccgccaaccctgtgatcaccgagagcaccgagaacagcaagatgatgctggaactggacccccccttcggcgactcctacatcgtgatcggcgtgggagagaagaagatcacccaccactggcacagaagcggcagcaccatcggcaaggcctttgaggctacagtgcggggagccaagagaatggccgtgctgggagataccgcctgggactttggctctgtgggcggagccctgaactctctg

SEQ ID NO: 4 - protein, 441 aa, Artificial Sequence
  Zika virus fusion sequence
MRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSL

SEQ ID NO: 5 - DNA, 1512 bp, Artificial Sequence
  Zika virus fusion sequence
atgcggtgtatcggcgtgtccaaccgggacttcgtggaaggcatgagcggcggcacatgggtggacgtggtgctggaacatggcggctgcgtgacagtgatggcccaggacaagcccaccgtggacatcgagctcgtgaccaccaccgtgtccaatatggccgaagtgcggagctactgctacgaggccagcatcagcgacatggccagcgacagcagatgccctacacagggcgaggcctacctggataagcagtccgacacccagtacgtgtgcaagcggaccctggtggatagaggctggggcaatggctgcggcctgtttggcaagggcagcctcgtgacctgcgccaagttcgcctgcagcaagaagatgaccggcaagagcatccagcccgagaacctggaataccggatcatgctgagcgtgcacggcagccagcactccggcatgatcgtgaacgacaccggccacgagacagacgagaaccgggccaaggtggaaatcacccccaacagccctagagccgaggccaccctgggcggctttggatctctgggactggactgcgagcccagaaccggcctggacttcagcgacctgtactacctgaccatgaacaacaagcactggctggtgcacaaagagtggttccacgacatccccctgccctggcatgccggcgctgatacaggcacaccccactggaacaacaaagaggctctggtggaattcaaggacgcccacgccaagcggcagaccgtggtggtgctgggatctcaggaaggcgccgtgcatacagctctggctggcgccctggaagccgaaatggatggcgccaaaggcagactgtccagcggccacctgaagtgccggctgaagatggacaagctgcggctgaagggcgtgtcctacagcctgtgtaccgccgccttcaccttcaccaagatccccgccgagacactgcacggcaccgtgactgtggaagtgcagtacgccggcaccgacggcccttgtaaagtgcctgctcagatggccgtggatatgcagaccctgacccccgtgggcagactgatcaccgccaaccctgtgatcaccgagagcaccgagaacagcaagatgatgctggaactggacccccccttcggcgactcctacatcgtgatcggcgtgggagagaagaagatcacccaccactggcacagaagcggcagcaccatcggcaaggcctttgaggctacagtgcggggagccaagagaatggccgtgctgggagataccgcctgggactttggctctgtgggcggagccctgaactctctgggcaagggaatccaccagatcttcggcgctgccttcaagagcctgttcggcggcatgagctggttcagccagatcctgatcggcaccctgctgatgtggctgggcctgaacaccaagaacggcagcatctccctgatgtgcctggctctgggaggcgtgctgatcttcctgagcacagccgtgtccgcc

SEQ ID NO: 6 - protein, 504 aa, Artificial Sequence
  Zika virus fusion sequence
MRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 7 - DNA, 1818 bp, Artificial Sequence
  Zika virus fusion sequence
acaagacggggcagcgcctactacatgtacctggacagaaacgacgccggcgaggccatcagcttccctaccacactgggcatgaacaagtgctacatccagatcatggacctgggccacatgtgcgacgccacaatgagctacgagtgccccatgctggacgagggcgtggaacccgacgatgtggactgctggtgcaacaccaccagcacctgggtggtgtacggcacctgtcaccacaagaagggcgaagccagacggtccagacgggccgtgacactgcctagccacagcaccagaaagctgcagacccggtcccagacctggctggaaagcagagagtacaccaagcacctgatccgggtggaaaactggatcttccggaaccccggctttgccctggccgctgctgctattgcttggctgctgggcagctccacctcccagaaagtgatctacctcgtgatgatcctgctgatcgcccctgcctacagcatccggtgtatcggcgtgtccaaccgggacttcgtggaaggcatgagcggcggcacatgggtggacgtggtgctggaacatggcggctgcgtgacagtgatggcccaggacaagcccaccgtggacatcgagctcgtgaccaccaccgtgtccaatatggccgaagtgcggagctactgctacgaggccagcatcagcgacatggccagcgacagcagatgccctacacagggcgaggcctacctggataagcagtccgacacccagtacgtgtgcaagcggaccctggtggatagaggctggggcaatggctgcggcctgtttggcaagggcagcctcgtgacctgcgccaagttcgcctgcagcaagaagatgaccggcaagagcatccagcccgagaacctggaataccggatcatgctgagcgtgcacggcagccagcactccggcatgatcgtgaacgacaccggccacgagacagacgagaaccgggccaaggtggaaatcacccccaacagccctagagccgaggccaccctgggcggctttggatctctgggactggactgcgagcccagaaccggcctggacttcagcgacctgtactacctgaccatgaacaacaagcactggctggtgcacaaagagtggttccacgacatccccctgccctggcatgccggcgctgatacaggcacaccccactggaacaacaaagaggctctggtggaattcaaggacgcccacgccaagcggcagaccgtggtggtgctgggatctcaggaaggcgccgtgcatacagctctggctggcgccctggaagccgaaatggatggcgccaaaggcagactgtccagcggccacctgaagtgccggctgaagatggacaagctgcggctgaagggcgtgtcctacagcctgtgtaccgccgccttcaccttcaccaagatccccgccgagacactgcacggcaccgtgactgtggaagtgcagtacgccggcaccgacggcccttgtaaagtgcctgctcagatggccgtggatatgcagaccctgacccccgtgggcagactgatcaccgccaaccctgtgatcaccgagagcaccgagaacagcaagatgatgctggaactggacccccccttcggcgactcctacatcgtgatcggcgtgggagagaagaagatcacccaccactggcacagaagcggcagcaccatcggcaaggcctttgaggctacagtgcggggagccaagagaatggccgtgctgggagataccgcctgggactttggctctgtgggcggagccctgaactctctg

SEQ ID NO: 8 - protein, 606 aa, Artificial Sequence
  Zika virus fusion sequence
TRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSL

SEQ ID NO: 9 - DNA, 2007 bp, Artificial Sequence
  Zika virus fusion sequence
acaagacggggcagcgcctactacatgtacctggacagaaacgacgccggcgaggccatcagcttccctaccacactgggcatgaacaagtgctacatccagatcatggacctgggccacatgtgcgacgccacaatgagctacgagtgccccatgctggacgagggcgtggaacccgacgatgtggactgctggtgcaacaccaccagcacctgggtggtgtacggcacctgtcaccacaagaagggcgaagccagacggtccagacgggccgtgacactgcctagccacagcaccagaaagctgcagacccggtcccagacctggctggaaagcagagagtacaccaagcacctgatccgggtggaaaactggatcttccggaaccccggctttgccctggccgctgctgctattgcttggctgctgggcagctccacctcccagaaagtgatctacctcgtgatgatcctgctgatcgcccctgcctacagcatccggtgtatcggcgtgtccaaccgggacttcgtggaaggcatgagcggcggcacatgggtggacgtggtgctggaacatggcggctgcgtgacagtgatggcccaggacaagcccaccgtggacatcgagctcgtgaccaccaccgtgtccaatatggccgaagtgcggagctactgctacgaggccagcatcagcgacatggccagcgacagcagatgccctacacagggcgaggcctacctggataagcagtccgacacccagtacgtgtgcaagcggaccctggtggatagaggctggggcaatggctgcggcctgtttggcaagggcagcctcgtgacctgcgccaagttcgcctgcagcaagaagatgaccggcaagagcatccagcccgagaacctggaataccggatcatgctgagcgtgcacggcagccagcactccggcatgatcgtgaacgacaccggccacgagacagacgagaaccgggccaaggtggaaatcacccccaacagccctagagccgaggccaccctgggcggctttggatctctgggactggactgcgagcccagaaccggcctggacttcagcgacctgtactacctgaccatgaacaacaagcactggctggtgcacaaagagtggttccacgacatccccctgccctggcatgccggcgctgatacaggcacaccccactggaacaacaaagaggctctggtggaattcaaggacgcccacgccaagcggcagaccgtggtggtgctgggatctcaggaaggcgccgtgcatacagctctggctggcgccctggaagccgaaatggatggcgccaaaggcagactgtccagcggccacctgaagtgccggctgaagatggacaagctgcggctgaagggcgtgtcctacagcctgtgtaccgccgccttcaccttcaccaagatccccgccgagacactgcacggcaccgtgactgtggaagtgcagtacgccggcaccgacggcccttgtaaagtgcctgctcagatggccgtggatatgcagaccctgacccccgtgggcagactgatcaccgccaaccctgtgatcaccgagagcaccgagaacagcaagatgatgctggaactggacccccccttcggcgactcctacatcgtgatcggcgtgggagagaagaagatcacccaccactggcacagaagcggcagcaccatcggcaaggcctttgaggctacagtgcggggagccaagagaatggccgtgctgggagataccgcctgggactttggctctgtgggcggagccctgaactctctgggcaagggaatccaccagatcttcggcgctgccttcaagagcctgttcggcggcatgagctggttcagccagatcctgatcggcaccctgctgatgtggctgggcctgaacaccaagaacggcagcatctccctgatgtgcctggctctgggaggcgtgctgatcttcctgagcacagccgtgtccgcc

SEQ ID NO: 10 - protein, 669 aa, Artificial Sequence
  Zika virus fusion sequence
TRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSA

SEQ ID NO: 11 - protein, 32 aa, Artificial Sequence
  Peptide
MDAMKRGLCCVLLLCGAVFVSPSQEIHARFRR

SEQ ID NO: 12 - protein, 27 aa, Artificial Sequence
  Peptide
SLLWGGVTVLAAMLIAGQVASSVVFLV

SEQ ID NO: 13 - protein, 165 aa, Artificial Sequence
  Zika virus
TRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYS

SEQ ID NO: 14 - DNA, 3750 bp, Chikungunya virus
atggaattcatccccacccagaccttctacaaccgcagataccagcccagaccctggacccccagacccaccatccaagtgatcagaccccggcctagaccccagagacaggctggacagctggctcagctgatctccgccgtgaacaagctgaccatgagagccgtgccccagcagaagcccagaaagaaccggaagaacaagaagcagaaacagaagcagcaggccccccagaacgaccccaagcagaagaagcagcctcctcagaagaaacccgcccagaagaagaaaaagcccggcagacgcgagcggatgtgcatgaagatcgagaacgactgcatcttcgaagtgaagcacgagggcaaagtgatgggctacgcctgcctcgtgggcgacaaagtgatgaagcccgcccacgtgaagggcaccatcgacaatgccgacctggccaagctggccttcaagcggagcagcaaatacgacctggaatgcgcccagatccccgtgcacatgaagtccgacgccagcaagttcacccacgagaagcccgagggctactacaactggcaccatggcgccgtgcagtacagcggcggcagattcacaatccccaccggcgctggaaagcctggcgatagcggcagacccatcttcgacaacaagggccgggtggtggccatcgtgctgggcggagctaatgagggcgccagaacagccctgagcgtcgtgacctggaacaaggacatcgtgaccaagatcacccccgagggcgccgaggaatggtccctggctatccctgtgatgtgcctgctggccaacaccaccttcccatgcagccagcccccttgcaccccttgctgctacgagaaagagcccgagagcaccctgcggatgctggaagataacgtgatgaggcccggctactaccagctgctgaaggcctccctgacctgcagccctcaccggcagagaagatccaccaaggacaacttcaacgtgtacaaggccaccagaccctacctggcccactgccctgattgtggcgagggccactcttgccactctcccgtggccctggaacggatcagaaacgaggccaccgacggcaccctgaagatccaggtgtccctgcagatcggcatcaagaccgacgacagccacgactggaccaagctgcggtacatggacaaccacatgcccgccgatgccgagagggcaggactgctcgtgcggacatctgccccctgtaccatcaccggcacaatgggccacttcatcctggccagatgccccaagggcgagacactgaccgtgggcttcaccgatggccggaagatcagccacagctgcacccaccccttccaccacgatcctcccgtgatcggcagagagaagttccacagcagaccccagcacggcaaagagctgccctgcagcacatacgtgcagagcacagccgccaccgccgaagagatcgaggtgcacatgcctcccgacacccccgacagaaccctgatgtctcagcagagcggcaacgtgaagatcaccgtgaacggccagaccgtgcggtacaagtgcaactgcggcggctccaatgagggcctgaccaccacagacaaagtgatcaacaactgcaagatcgaccagtgccacgccgccgtgaccaaccacaagaagtggcagtacaacagccccctggtgcccagaaatgccgagctgggcgaccggaagggcaagatccacatccctttccccctggccaacgtgacctgccgggtgcccaaagccagaaaccccaccgtgacctacggcaagaaccaagtgattatgctgctgtaccccgaccaccccaccctgctgagctacagaaacatgggcgaggaacccaactaccacgaagagtgggtcacccacaagaaagaagtgcggctgaccgtgcccaccgagggcctggaagtgacctggggcaacaacgagccctacaagtactggccccagctgagcaccaatggcacagcccacggacacccccacgagatcatcctgtactactacgagctgtaccctaccatgaccgtcgtgatcgtgtctgtggccagcttcgtgctgctgagcatggtgggaacagccgtgggcatgtgtatgtgcgccagacggcggtgcatcaccccttacgaactgacccctggcgccaccgtgccctttctgctgagcctgatctgctgcatccggaccgccaaggccgccacctattatgaggccgctgcctacctgtggaacgagcagcagcccctgttttggctgcaagccctgattcctctggccgccctgatcgtgctgtgcaactgcctgagactgctgccctgctgctgcaagaccctggcctttctggccgtgatgagcatcggagcccacaccgtgtctgcctacgagcacgtgaccgtgatccccaacacagtgggcgtgccctacaaaaccctcgtgaacagacccggctacagccctatggtgctggaaatggaactgctgagcgtgaccctggaacccaccctgagcctggactacatcacatgcgagtacaagacagtgatccctagcccctacgtgaagtgctgcggcaccgccgagtgcaaggacaagagcctgcccgactacagctgcaaggtgttcaccggcgtgtaccccttcatgtggggcggagcctactgcttttgcgacgccgagaacacacagctgagcgaggcccacgtggaaaagagcgagagctgcaaaaccgagttcgccagcgcctacagggcccacacagcctctgcctctgccaagctgagagtgctgtaccagggcaacaatatcaccgtggccgcctacgccaacggcgaccatgccgtgacagtgaaggacgccaagttcatcgtgggccccatgagcagcgcctggacacccttcgataacaagattgtggtgtataagggggatgtgtacaacatggactaccccccctttggcgccggacggcctggacagtttggcgacatccagagcagaacccctgagagcaaggacgtgtacgccaacacccagctggtgctgcagaggcctgcagccggaacagtgcacgtgccatactctcaggcccccagcggcttcaagtattggctgaaagagagaggcgccagcctgcagcataccgcccctttcggctgtcagatcgccaccaatcctgtgcgggccgtgaattgcgccgtgggaaacatccccatcagcatcgacatccccgacgccgccttcaccagagtggtggatgcccctagcctgaccgacatgagctgcgaagtgcccgcctgcacacacagcagcgattttggcggagtggccatcattaagtacgccgcctccaagaaaggcaagtgtgccgtgcacagcatgaccaacgccgtgacaatccgcgaggccgagattgaggtggaaggcaacagccagctgcagatcagcttctccacagccctggccagcgccgagttcagagtgcaagtgtgcagcacccaggtgcactgcgctgccgcttgtcacccccccaaggaccacatcgtgaactaccctgccagccacaccaccctgggcgtgcaggatatcagcaccaccgccatgtcctgggtgcagaaaatcacagggggcgtgggactgatcgtggccgtggctgctctgattctgattgtggtgctgtgcgtgtccttcagccggcactgatga

SEQ ID NO: 15 - protein, 1248 aa, Chikungunya virus
MEFIPTQTFYNRRYQPRPWTPRPTIQVIRPRPRPQRQAGQLAQLISAVNKLTMRAVPQQKPRKNRKNKKQKQKQQAPQNDPKQKKQPPQKKPAQKKKKPGRRERMCMKIENDCIFEVKHEGKVMGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWSLAIPVMCLLANTTFPCSQPPCTPCCYEKEPESTLRMLEDNVMRPGYYQLLKASLTCSPHRQRRSTKDNFNVYKATRPYLAHCPDCGEGHSCHSPVALERIRNEATDGTLKIQVSLQIGIKTDDSHDWTKLRYMDNHMPADAERAGLLVRTSAPCTITGTMGHFILARCPKGETLTVGFTDGRKISHSCTHPFHHDPPVIGREKFHSRPQHGKELPCSTYVQSTAATAEEIEVHMPPDTPDRTLMSQQSGNVKITVNGQTVRYKCNCGGSNEGLTTTDKVINNCKIDQCHAAVTNHKKWQYNSPLVPRNAELGDRKGKIHIPFPLANVTCRVPKARNPTVTYGKNQVIMLLYPDHPTLLSYRNMGEEPNYHEEWVTHKKEVRLTVPTEGLEVTWGNNEPYKYWPQLSTNGTAHGHPHEIILYYYELYPTMTVVIVSVASFVLLSMVGTAVGMCMCARRRCITPYELTPGATVPFLLSLICCIRTAKAATYYEAAAYLWNEQQPLFWLQALIPLAALIVLCNCLRLLPCCCKTLAFLAVMSIGAHTVSAYEHVTVIPNTVGVPYKTLVNRPGYSPMVLEMELLSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFIVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSLTDMSCEVPACTHSSDFGGVAIIKYAASKKGKCAVHSMTNAVTIREAEIEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 16 - DNA, 2976 bp, Chikungunya virus
gaggaatggtccctggctatccctgtgatgtgcctgctggccaacaccaccttcccatgcagccagcccccttgcaccccttgctgctacgagaaagagcccgagagcaccctgcggatgctggaagataacgtgatgaggcccggctactaccagctgctgaaggcctccctgacctgcagccctcaccggcagagaagatccaccaaggacaacttcaacgtgtacaaggccaccagaccctacctggcccactgccctgattgtggcgagggccactcttgccactctcccgtggccctggaacggatcagaaacgaggccaccgacggcaccctgaagatccaggtgtccctgcagatcggcatcaagaccgacgacagccacgactggaccaagctgcggtacatggacaaccacatgcccgccgatgccgagagggcaggactgctcgtgcggacatctgccccctgtaccatcaccggcacaatgggccacttcatcctggccagatgccccaagggcgagacactgaccgtgggcttcaccgatggccggaagatcagccacagctgcacccaccccttccaccacgatcctcccgtgatcggcagagagaagttccacagcagaccccagcacggcaaagagctgccctgcagcacatacgtgcagagcacagccgccaccgccgaagagatcgaggtgcacatgcctcccgacacccccgacagaaccctgatgtctcagcagagcggcaacgtgaagatcaccgtgaacggccagaccgtgcggtacaagtgcaactgcggcggctccaatgagggcctgaccaccacagacaaagtgatcaacaactgcaagatcgaccagtgccacgccgccgtgaccaaccacaagaagtggcagtacaacagccccctggtgcccagaaatgccgagctgggcgaccggaagggcaagatccacatccctttccccctggccaacgtgacctgccgggtgcccaaagccagaaaccccaccgtgacctacggcaagaaccaagtgattatgctgctgtaccccgaccaccccaccctgctgagctacagaaacatgggcgaggaacccaactaccacgaagagtgggtcacccacaagaaagaagtgcggctgaccgtgcccaccgagggcctggaagtgacctggggcaacaacgagccctacaagtactggccccagctgagcaccaatggcacagcccacggacacccccacgagatcatcctgtactactacgagctgtaccctaccatgaccgtcgtgatcgtgtctgtggccagcttcgtgctgctgagcatggtgggaacagccgtgggcatgtgtatgtgcgccagacggcggtgcatcaccccttacgaactgacccctggcgccaccgtgccctttctgctgagcctgatctgctgcatccggaccgccaaggccgccacctattatgaggccgctgcctacctgtggaacgagcagcagcccctgttttggctgcaagccctgattcctctggccgccctgatcgtgctgtgcaactgcctgagactgctgccctgctgctgcaagaccctggcctttctggccgtgatgagcatcggagcccacaccgtgtctgcctacgagcacgtgaccgtgatccccaacacagtgggcgtgccctacaaaaccctcgtgaacagacccggctacagccctatggtgctggaaatggaactgctgagcgtgaccctggaacccaccctgagcctggactacatcacatgcgagtacaagacagtgatccctagcccctacgtgaagtgctgcggcaccgccgagtgcaaggacaagagcctgcccgactacagctgcaaggtgttcaccggcgtgtaccccttcatgtggggcggagcctactgcttttgcgacgccgagaacacacagctgagcgaggcccacgtggaaaagagcgagagctgcaaaaccgagttcgccagcgcctacagggcccacacagcctctgcctctgccaagctgagagtgctgtaccagggcaacaatatcaccgtggccgcctacgccaacggcgaccatgccgtgacagtgaaggacgccaagttcatcgtgggccccatgagcagcgcctggacacccttcgataacaagattgtggtgtataagggggatgtgtacaacatggactaccccccctttggcgccggacggcctggacagtttggcgacatccagagcagaacccctgagagcaaggacgtgtacgccaacacccagctggtgctgcagaggcctgcagccggaacagtgcacgtgccatactctcaggcccccagcggcttcaagtattggctgaaagagagaggcgccagcctgcagcataccgcccctttcggctgtcagatcgccaccaatcctgtgcgggccgtgaattgcgccgtgggaaacatccccatcagcatcgacatccccgacgccgccttcaccagagtggtggatgcccctagcctgaccgacatgagctgcgaagtgcccgcctgcacacacagcagcgattttggcggagtggccatcattaagtacgccgcctccaagaaaggcaagtgtgccgtgcacagcatgaccaacgccgtgacaatccgcgaggccgagattgaggtggaaggcaacagccagctgcagatcagcttctccacagccctggccagcgccgagttcagagtgcaagtgtgcagcacccaggtgcactgcgctgccgcttgtcacccccccaaggaccacatcgtgaactaccctgccagccacaccaccctgggcgtgcaggatatcagcaccaccgccatgtcctgggtgcagaaaatcacagggggcgtgggactgatcgtggccgtggctgctctgattctgattgtggtgctgtgcgtgtccttcagccggcactgatga

SEQ ID NO: 17 - protein, 991 aa, Chikungunya virus
MEEWSLAIPVMCLLANTTFPCSQPPCTPCCYEKEPESTLRMLEDNVMRPGYYQLLKASLTCSPHRQRRSTKDNFNVYKATRPYLAHCPDCGEGHSCHSPVALERIRNEATDGTLKIQVSLQIGIKTDDSHDWTKLRYMDNHMPADAERAGLLVRTSAPCTITGTMGHFILARCPKGETLTVGFTDGRKISHSCTHPFHHDPPVIGREKFHSRPQHGKELPCSTYVQSTAATAEEIEVHMPPDTPDRTLMSQQSGNVKITVNGQTVRYKCNCGGSNEGLTTTDKVINNCKIDQCHAAVTNHKKWQYNSPLVPRNAELGDRKGKIHIPFPLANVTCRVPKARNPTVTYGKNQVIMLLYPDHPTLLSYRNMGEEPNYHEEWVTHKKEVRLTVPTEGLEVTWGNNEPYKYWPQLSTNGTAHGHPHEIILYYYELYPTMTVVIVSVASFVLLSMVGTAVGMCMCARRRCITPYELTPGATVPFLLSLICCIRTAKAATYYEAAAYLWNEQQPLFWLQALIPLAALIVLCNCLRLLPCCCKTLAFLAVMSIGAHTVSAYEHVTVIPNTVGVPYKTLVNRPGYSPMVLEMELLSVTLEPTLSLDYITCEYKTVIPSPYVKCCGTAECKDKSLPDYSCKVFTGVYPFMWGGAYCFCDAENTQLSEAHVEKSESCKTEFASAYRAHTASASAKLRVLYQGNNITVAAYANGDHAVTVKDAKFIVGPMSSAWTPFDNKIVVYKGDVYNMDYPPFGAGRPGQFGDIQSRTPESKDVYANTQLVLQRPAAGTVHVPYSQAPSGFKYWLKERGASLQHTAPFGCQIATNPVRAVNCAVGNIPISIDIPDAAFTRVVDAPSLTDMSCEVPACTHSSDFGGVAIIKYAASKKGKCAVHSMTNAVTIREAEIEVEGNSQLQISFSTALASAEFRVQVCSTQVHCAAACHPPKDHIVNYPASHTTLGVQDISTTAMSWVQKITGGVGLIVAVAALILIVVLCVSFSRH

SEQ ID NO: 18 - DNA, 4641 bp, Artificial Sequence
  Zika virus fusion sequence
accatgaagaaccccaagaagaagtccggcggcttccggatcgtgaacatgctgaaacggggcgtggccagagtgaaccctctgggcggactgaagagactgcctgccggactgctgctgggccacggccctattagaatggtgctggccatcctggcctttctgcggttcaccgccatcaagcctagcctgggcctgatcaacagatggggcagcgtgggcaagaaagaagccatggaaatcatcaagaagttcaagaaagacctggccgccatgctgcggatcatcaacgcccggaaagagcggaagcggagaggcgccgataccagcatcggcatcattggcctgctgctgaccacagccatggccgccgagatcaccagaagaggcagcgcctactacatgtacctggacagaagcgacgccggcaaggccatcagctttgccacaaccctgggcgtgaacaagtgccacgtgcagatcatggacctgggccacatgtgcgacgccacaatgagctacgagtgccccatgctggacgagggcgtggaacccgacgatgtggactgctggtgcaacaccaccagcacctgggtggtgtacggcacctgtcaccacaagaagggcgaggccagacggtctagaagggccgtgacactgcctagccacagcacccggaagctgcagaccagaagccagacctggctggaaagcagagagtacaccaagcacctgatcaaggtggaaaactggatcttccggaaccccggcttcgccctggctgccgtggctattgcttggctgctgggaagcagcaccagccagaaagtgatctacctcgtgatgatcctgctgatcgcccctgcctacagcatccggtgtatcggcgtgtccaaccgggacttcgtggaaggcatgagcggcggcacatgggtggacgtggtgctggaacatggcggctgcgtgacagtgatggcccaggacaagcccaccgtggacatcgagctcgtgaccaccaccgtgtccaatatggccgaagtgcggagctactgctacgaggccagcatcagcgacatggccagcgacagcagatgccctacacagggggaggcctacctggataagcagtccgacacccagtacgtgtgcaagcggaccctggtggatagaggctggggcaatggctgcggcctgtttggcaagggcagcctcgtgacctgcgccaagttcacctgtagcaagaagatgaccggcaagagcatccagcccgagaacctggaataccggatcatgctgagcgtgcacggctcccagcacagcggcatgatcgtgaatgacatcggccacgagacagacgagaaccgggccaaagtggaagtgacccccaacagccctagagccgaggccacactgggcggctttggatctctgggcctggactgcgagcctagaaccggcctggatttcagcgacctgtactacctgaccatgaacaacaaacactggctggtgcacaaagagtggttccacgacatccccctgccctggcatgctggcgctgatacaggcaccccccactggaacaacaaagaggccctggtggagttcaaggacgcccacgccaagaggcagaccgtggtggtgctgggatctcaggaaggcgccgtgcatacagctctggctggcgccctggaagccgaaatggatggcgctaagggccggctgtttagcggccacctgaagtgccggctgaagatggacaagctgcggctgaagggcgtgtcctacagcctgtgtaccgccgccttcaccttcaccaaggtgcccgccgaaaccctgcacggcacagtgactgtggaagtgcagtacgccggcaccgacggcccttgtaaagtgcctgctcagatggccgtggatatgcagaccctgacccccgtgggcagactgatcaccgccaaccctgtgatcaccgagagcaccgagaacagcaagatgatgctggaactggacccccccttcggcgactcctacatcgtgatcggcgtgggagacaagaagatcacccaccactggcaccgcagcggcagcacaatcggaaaggccttcgaagccacagtgcggggagccaagagaatggccgtgctgggcgataccgcctgggattttggctctgtgggcggcgtgttcaactccctgggcaagggaatccaccagatcttcggagccgcctttaagagcctgttcggcggcatgagctggttcagccagatcctgatcggcaccctgctcgtgtggctgggactgaacaccaagaacggcagcatctccctgacctgcctggctctggggggagtgatgatcttcctgagcaccgccgtgtccgcccctagcgaagtgctgacagccgtgggactgatctgcgctctggcaggcggattcgccaaggccgacattgagatggccggacccatggctgctgtgggactgctgattgtgtcctacgtggtgtccggcaagtctgtggacatgtacatcgagagagccggcgacatcacctgggagaaggacgccgaagtgacaggcaacagccccagactggacgtggccctggatgagagcggcgatttcagtctggtggaagaggacggccctcccatgcgcgagatcattctgaaagtggtgctgatggcaatctgcgggatgaaccctatcgccatccccttcgctgccggcgcttggtacgtgtacgtgaaaacaggcaagcggagcggagccctgtgggatgtgcctgcccccaaagaagtgaagaaaggcgagacaaccgacggcgtgtacagagtgatgacccgcagactgctgggcagcacacaagtgggagtgggcgtgatgcaggaaggggtgttccacaccatgtggcacgtgaccaaaggcgccgctctgagatctggcgagggcaggctggatccttactggggcgacgtgaagcaggacctggtgtcctattgcggcccttggaagctggacgccgcttgggatggactgagcgaggtgcagctgctggctgtgcctcctggcgagagggccagaaacatccagaccctgccaggcatcttcaagaccaaggacggggacatcggcgccgtggctctggattatcctgccggcacaagcggctcccccatcctggacaagtgtggcagagtgatcggcctgtacggcaacggcgtcgtgatcaagaatggcagctatgtgtccgccatcacccagggcaagcgggaagaggaaacccctgtggaatgcttcgagccctccatgctgaagaaaaagcagctgaccgtgctggacctgcaccctggcgccggaaaaaccagaagggtgctgcctgagatcgtgcgggaagccatcaagaaacggctgagaaccgtgatcctggcccccaccagagtggtggctgccgagatggaagaagccctgagaggactgcccgtgcggtacatgacaaccgccgtgaacgtgacccactctggcaccgagatcgtggatctgatgtgtcacgccaccttcacaagccggctgctgcagcccatccgggtgcccaactacaacctgtacatcatggacgaggcccacttcaccgaccccagctccattgccgccagaggctacatcagcacacgggtggaaatgggcgaagctgccgccatcttcatgaccgccacacctcccggaaccagggacgccttccccgacagcaactcccctatcatggacaccgaggtggaagtgcccgagagagcctggtccagcggcttcgactgggtcacagatcactccggcaagaccgtgtggttcgtgccctctgtgcggaacggcaatgagatcgccgcctgtctgacaaaggccgggaagagagtgatccagctgagccgcaagaccttcgagacagagttccagaaaacaaagaaccaggaatgggatttcgtgatcaccacagacatctccgagatgggcgccaacttcaaggccgatcgcgtgatcgacagccggcggtgtctgaagcccgtgattctggacggcgaaagagtgattctggccggacctatgcccgtgacccatgcctctgccgctcagagaagaggccggatcggcagaaaccccaacaagcccggcgacgagtatatgtacggcggaggctgcgccgagactgacgaggatcatgcccattggctggaagccagaatgctgctggacaacatatacctgcaggacggcctgatcgcctccctgtacagacccgaggctgacaaagtggctgccatcgagggcgagttcaagctgaggaccgagcagagaaagacatttgtggaactgatgaagcggggcgacctgcctgtgtggctggcctatcaggtggcatctgccggcatcacctacaccgacagacggtggtgcttcgacggcaccaccaacaacaccatcatggaagatagcgtgccagccgaagtgtggaccaaatacggcgagaagcgcgtgctgaagccccggtggatggacgccagagtgtgttctgatcacgccgcactgaagtccttcaaagagttcgccgctggcaagtgatgagcggccgctcgagtacgtctg

SEQ ID NO: 19 - protein, 1537 aa, Artificial Sequence
  Zika virus fusion sequence
MKNPKKKSGGFRIVNMLKRGVARVNPLGGLKRLPAGLLLGHGPIRMVLAILAFLRFTAIKPSLGLINRWGSVGKKEAMEIIKKFKKDLAAMLRIINARKERKRRGADTSIGIIGLLLTTAMAAEITRRGSAYYMYLDRSDAGKAISFATTLGVNKCHVQIMDLGHMCDATMSYECPMLDEGVEPDDVDCWCNTTSTWVVYGTCHHKKGEARRSRRAVTLPSHSTRKLQTRSQTWLESREYTKHLIKVENWIFRNPGFALAAVAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIVNDIGHETDENRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKVPAQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSAPSEVLTAVGLICALAGGFAKADIEMAGPMAAVGLLIVSYVVSGKSVDMYIERAGDITWEKDAEVTGNSPRLDVALDESGDFSLVEEDGPPMREIILKVVLMAICGMNPIAIPFAAGAWYVYVKTGKRSGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEETPVECFEPSMLKKKQLTVLDLHPGAGKTRRVLPEIVREAIKKRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKNQEWDFVITTDISEMGANFKADRVIDSRRCLKPVILDGERVILAGPMPVTHASAAQRRGRIGRNPNKPGDEYMYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTKYGEKRVLKPRWMDARVCSDHAALKSFKEFAAGK

SEQ ID NO: 20 - protein, 14 aa, Artificial Sequence
  Zika virus
YCYEASISDMASDS

SEQ ID NO: 21 - protein, 14 aa, Dengue virus
LCIEAKISNTTTDS

SEQ ID NO: 22 - protein, 14 aa, Dengue virus
YCIEAKLTNTTTDS

SEQ ID NO: 23 - protein, 14 aa, Dengue virus
LCIEGKITNITTDS

SEQ ID NO: 24 - protein, 14 aa, Dengue virus
YCIEASISNITTAT

SEQ ID NO: 25 - protein, 14 aa, Artificial Sequence
  Bagaza virus
YCYEPKITDVSTEP

SEQ ID NO: 26 - protein, 14 aa, Artificial Sequence
  Kedougou virus
YCYEATISDTVMET

SEQ ID NO: 27 - protein, 11 aa, Artificial Sequence
  Zika virus
PVGRLITANPV

SEQ ID NO: 28 - protein, 11 aa, Dengue virus
QNGRLITANPI

SEQ ID NO: 29 - protein, 11 aa, Dengue virus
VLGRLITVNPI

SEQ ID NO: 30 - protein, 11 aa, Dengue virus
HNGRLITANPV

SEQ ID NO: 31 - protein, 11 aa, Artificial Sequence
  Bagaza virus
PIGRLITVNPY

SEQ ID NO: 32 - protein, 11 aa, Artificial Sequence
  Kedougou virus
NVGRLVTANPI